(19) United States
(12) Patent Application Publication
(10) Pub. No.: US 2020/0316172 A1
(43) Pub. Date: Oct. 8, 2020

(54) MATURATION OF MUCOSAL DEFENSE AND GUT/LUNG FUNCTION IN THE PRETERM INFANT

(71) Applicant: DEFENSIN THERAPEUTICS APS, Copenhagen N (DK)

(72) Inventor: Peter Norkild, Kabenhavn K. (DK)

(21) Appl. No.: 16/761,980

(22) PCT Filed: Nov. 9, 2018
(86) PCT No.: PCT/EP2018/080814

§ 371 Date: May 6, 2020

(30) Foreign Application Priority Data
Nov. 10, 2017 (EP) 17201002.7

(51) Int. Cl. A61K 38/17; A61P 11/00; A61P 1/00

(52) U.S. Cl. CPC A61K 38/1729; A61K 9/0078; A61P 1/00; A61P 11/00

(57) ABSTRACT

The present invention relates to methods for maturing the mucosal defense and rebalancing the immune system preventing a cytokine storm; treatment or prevention of neonatal sepsis, necrotizing enterocolitis, acute and prolonged diarrhea, short bowel syndrome, respiratory illness, respiratory infection, respiratory failure, impaired neurodevelopment and extra uterine growth restriction, the method comprising oral and/or intrapulmonary and/or subcutaneous administration of at least one antimicrobial peptide selected from the group consisting of α-defensins, β-defensins, cathelicidins, lactoferrins/lactoferricins and lysozymes and/or GLP-2 or GLP-2 analogs in a preterm infant or a mother about to give birth to a preterm infant.

### FIELD OF THE INVENTION

The present invention relates to methods for treatment or prevention of complications associated with preterm birth such as sepsis, respiratory illness, necrotizing enterocolitis, short bowel syndrome, impaired neurodevelopment and extrauterine growth restriction based on maturation and normalization of gut and lung microbiota and rebalancing of the immune system with decreased incidence of necrotizing enterocolitis, increased mucosal proportion, increased height of the intestinal villi and density of goblet cells, higher digestive brush border enzyme activity, lower intestinal myeloperoxidase activity; reduced airway hyper responsiveness, increased pulmonary compliance, reduced lung inflammation, reduced perivascular and peribronchial inflammation, reduced inflammatory cell count in bronchoalveolar fluid and reduced cytokine production by administration of one or more mammalian antimicrobial peptides such as defensins, cathelicidin, lactoferrin/lactoferricin and/or lysozymes.

### BACKGROUND

Extreme Low Birth Weight (ELBW) Infants (Birthweight<1.000 Gram).

At birth, all newborn infants have to adapt to the dramatic transition from a sterile life in the mother to an environment where environmental microbes colonize all epithelial surfaces of the body (e.g. gut, lungs, skin) within the first days and weeks after birth. Adaptation requires a well-developed innate immune system at epithelial surfaces, that allows tolerance to harmless or beneficial microbes and exclusion of harmful pathogens. After a normal gestation length, delivery process, maternal care and hygiene, newborn infants adapt well, but a markedly higher prevalence of infection, sepsis and inflammatory disorders in early life nevertheless shows that infants and children are very sensitive to the dramatic microbiological and immunological transition at birth.

Of the 15 million infants born preterm each year worldwide, more than 1 million die (Strunk, 2014). Many of these deaths relates to their immature ability to fight bacterial colonization at the gut, lung and skin surfaces. Infections are related to immature lung, skin and gut barrier functions and especially tolerance to the great number of bacteria along the gut surface from a few days after birth (≈10 billion) is a problem. These infections induce impaired gut function and pathogens/toxins may translocate into the blood stream, create sepsis and inflammatory reactions throughout the body. Morbidity is inversely related to gestational age, however there is no gestational age, including term that is wholly exempt. Although most organs are immature, the brain and lung are especially susceptible to the consequences of preterm birth, which leads to high rates of long-term neurological and health problems. In a review, more than half of ELBW children were readmitted to hospital at least once in the first 1-2 years of life, mostly as a result of respiratory illnesses (Doyle, 2003). Most studies of very low birth weight (VLBW) infants show continued sequelae such as cognitive deficits, academic underachievement, grade failures, and the need for increased remedial assistance during mid-childhood and adolescence (Saigal, 2008).

Lung Function

Normal lung development follows a series of orchestrated events. Premature birth interrupts normal in utero lung development, which results in significant alterations in lung function and physiology with an early transition from the hypoxic intrauterine environment to a comparatively hyperoxic environment (Colin, 2010) leading to respiratory distress syndrome, bronchopulmonary dysplasia, patent ductus arteriosus and chronic lung disease. Total lung volume undergoes rapid changes during the last trimester of gestation. Calculations by Langston, 1984 revealed that at 30 week's, the lung volume is only 34% of the ultimate lung volume at mature birth. Immaturity of innate immunity contributes to the increased susceptibility of human neonates to infection. The lung is a major portal of entry for potential pathogens in the neonate. Alfa defensins, beta defensins and cathelicidin participate in the pulmonary innate immunity. Very few studies exist describing the role of antimicrobial peptides in neonate development but Tirone, 2010 found that preterm infants even with a gestational age of 30 weeks or less were able to produce alfa-defensins and Starner, 2015 found that treatment with the anti-inflammatory corticosteroid dexamethasone resulted in a significant decrease in hBD-2 expression.

Infection and Sepsis

Infections can be congenital or acquired. The congenital infections consist of pneumonia and chorioamnionitis attributable to maternal enteric organisms. The acquired infections are mainly caused by candida and nosocomial bacteria. Infection of the amniotic fluid leading to pneumonia is the major cause of death in the ELBW infants (Barton, 1999).

Bacterial translocation and endotoxemia caused by the loss of gut mucosal barrier function have been linked with organ injury, systemic inflammation and multiple organ failure. The gut mucosal barrier defense of premature and immune-incompetent infants offers imperfect protection against bacterial translocation and endotoxemia. Gastrointestinal distension is a common event in premature infants that may impair gut barrier function and hence initiate bacterial translocation (Sharma, 2007). The risk of sepsis is inversely related to birth weight and gestational age. Infants with sepsis are at increased risk for a number of neonatal morbidities, for prolonged hospitalization and for death (Stoll, 2003). In the ELBW infant, invasive candidiasis leads to poor neurodevelopmental outcomes. Invasive candidiasis is the second most common cause of infectious disease related death in the extremely premature infant. Invasive candidiasis was found in 1.5% of VLBW infants. CNS candidiasis is under-investigated and difficult to diagnose but portends a very poor outcome (Barton, 2014). Previous treatment with antibiotics, presence of a central catheter or endotracheal tube were found to be strongly associated with invasive candidiasis (Benjamin, 2010). In the past decade, several immune interventions aimed to prevent or improve the outcome of neonatal sepsis, such as colony-stimulating growth factors and intravenous immunoglobulin have been unsuccessful (Strunk, 2014).

Necrotizing Enterocolitis (NEC)

is a devastating gut inflammatory disease that occurs in 1-7% of all hospitalized preterm infants and is the second highest cause of mortality in preterms (Anand, 2007). The disease is characterized by bacterial overgrowth, dysregulated immunity and intestinal necrosis and is particularly prevalent in infants lacking breast-feeding due to the common maternal illness and lack of lactation. NEC appears to develop in the stressed newborn suffering from an immature innate and adaptive immune system after a disruption in the intestinal barrier and translocation of bacterial endotoxins (Sharma, 2007). The infants that survive NEC are at high risk of developing long-term complications, particularly neurodevelopmental impairment e.g. cerebral palsy (Strunk, 2014), as well as short bowel syndrome and impaired growth (Patel, 2012). Humoral mediators, including pro-inflammatory cytokines are likely to be key mediators in the pathogenesis of cerebral injury (Strunk, 2014).

NEC and sepsis often appear together but it remains unclear how gut bacterial dyscolonization and impaired immunity directly relate to bacterial translocation and the common complications of preterm birth, such as respiratory failure, cardiovascular abnormalities, impaired immunity, NEC and brain dysfunction (Sangild, 2013). The role of defensins in NEC is unclear. Salzman, 1998 originally found the expression of defensins and the number of Paneth cells to be higher in the neonatal gut of NEC patients. Puiman, 2011 on the other hand found that Paneth cell abundance in the small intestine was not significantly different in acute NEC from preterm controls. Richter, 2010 speculated that low fecal hBD-2 may be a risk factor in preterm infants to develop neonatal enteric disease, such as necrotizing enterocolitis. Jenke, 2012 found that low hBD-2 expression was associated with TLR4 expression, suggesting an indaquate response to luminal bacteria, possibly predisposing those infants to the development of NEC. Risk factors for NEC are gut immaturity, dysmolality, abnormal microbiota and increased permeability.

Antibiotics have been shown to modulate intestinal immunity and prevent NEC in a preterm neonatal piglet model (Jensen, 2014) and hBD-3 has been shown to decrease the incidence of NEC and survival in a neonatal rat model (Sheng, 2014). Manzoni, 2014 found the incidence of NEC to be significantly lower in VLBW infants treated with bovine lactoferrin plus Lactobacillus rhamnosus versus placebo.

Impaired Neurodevelopment

Several studies in preterm infants show an association between late-onset sepsis and adverse neurodevelopmental outcomes in childhood, with repeated infections and Gram-negative pathogens conferring the highest risk. The association between sepsis and cerebral injury seems however to be largely independent of the bacterial species involved suggesting that a detrimental final common pathway can be activated by diverse initial host-microbe interactions (Strunk et. 2014). Examples of neurodevelopmental impairments in the early years are cerebral palsy, mental retardation, and sensory impairments such as visual and auditory deficits (Saigal, 2008).

The commonest lesion associated with inflammation in preterm infants is white-matter injury, which is characterized by focal cystic periventricular leukomalacia, diffuse necrosis or both (Strunk, 2014). Humoral mediators, including proinflammatory cytokines such as IL-1 and IL-6 and chemokines such as CXCL-8, TNF-α, type I and II interferons, and reactive oxygen species are likely to be key mediators in the pathogenesis of cerebral injury. Detrimental neurotoxic effects are not only induced by direct host-microbe interaction, but might also be generated by exposure to perinatal inflammation, activation of fetal or neonatal immune cells triggered by bacterial products that activate pattern recognition receptors, or maternal proinflammatory mediators that cross the placenta (Girard, 2009).

Extrauterine Growth Restriction

Extrauterine growth restriction (infants with a weight below the 10th percentile between birth and discharge) in VLBW infants affects their growth and development prognoses as well as their incidence of adult diseases (Sakurai, 2008). Sakurai found the incidence of extrauterine growth restriction to be 28%, 34% and 16% for weight, length, and head circumference, respectively. For each growth parameter, the incidence of extra uterine growth restriction increased with decreasing gestation and birth weight. In a cohort of infants weighing 600 g or less, it was reported that, at hospital discharge and 2 years of age, 94% were below the 10^{th} percentile for weight, length and head circumference. Abnormal neurodevelopmental outcome was found in 90% of survivors (Sweet, 2003).

Neonatal Microbiome

The microbiome evolves within a healthy host from birth to death, constantly fine-tuning it to maintain homeostatic balance with the host's immune system. The first and most important contribution to the genesis of the microbiome is vertical transmission of maternal microbiota. Colonization of mucosa in the digestive, respiratory, urogenital tracts as well as the skin begins at or perhaps even before, the time of birth when a newborn is exposed to a mother's microbiota. Recent studies suggest the presence of a microbiome within the placenta as well as fetal meconium, suggesting that the colonization process begins well before delivery.

The gastrointestinal tract has the greatest diversity and abundance of microbes. More than 99% of the gut microbes are anaerobes. Infants born via vaginal delivery have intestinal colonization reflective of maternal vaginal flora such as Lactobacillus and Prevotella species. Infants born via Caesarian delivery are colonized by epidermal rather than vaginal species such as Clostridium, Staphylococcus, Propionebacterium and Corynebacterium. Further development of the neonatal gut microbiome after birth, regardless of mode of delivery is governed by interaction between the microbiota and the host's immune system. The progression of how this evolves remains incompletely characterized. Nutrition, be it in breast milk or formula, has been demonstrated to play a major role in early colonization patterns of the neonatal gut microbiota (Gritz and Bandari, 2015). Interestingly, even relatively small amounts of formula supplementation of breast-fed infants will result in shifts from a breast-fed to a formula-fed pattern (Guaraldi and Salvatori, 2012).

Preterm infants, especially ELBW infants, are at a disadvantage when it comes to development of a healthy microbiome. Factors contributing to this are not limited to their gut immaturity, but also include preterm ruptured membranes, maternal infection, increased incidence of Caesarian delivery, perinatal and postnatal broad spectrum antibiotic exposure as well as exposure to other gut-modifying medications (Gritz and Bandari, 2015). A 2007 study by Butel found that healthy full-term breastfed infants are colonized by Bifidobacterium by day 7 of life, whereas preterm infants are not. Interestingly, they also suggest that there may be gestational age thresholds for colonization with certain microbes—33 weeks appears to be the milestone for appearance of Bifidobacterium, the organism most commonly implicated in development and maintenance of a healthy microbiome.

Colostrum

Human milk protects against infections in the breastfed offspring mainly via the secretory IgA antibodies, but also most likely several other factors like the bactericidal lactoferrin. Protection against infections has been well evidenced during lactation against, e.g. acute and prolonged diarrhea, respiratory tract infections, otitis media, urinary tract infection, neonatal sepsis and NEC (Hanson, 1998).

Consumption of human milk provides passive immunity to the newborn GI tract through a large number of soluble and cellular components. Milk contains antimicrobial proteins and peptides including lactoferrin, lysozyme and defensins, which modulate the gut microbiome. In addition to antimicrobial activity, breast milk may contribute to maintenance of the barrier function of the GI tract, through growth factors and cytokines, or to the defense of the GI epithelium through soluble pattern recognition receptors such as secretory IgA and soluble CD-14 receptor, which may prevent bacterial attachment to enteric tissues through steric hindrance (Trend, 2016). The concentration of protein and immune factors such as sIgA and leucocytes are reported to be altered by maternal age, mode of delivery, the volume of milk produced, smoking, BMI, parity and maternal infection. Many of these maternal parameters are also risk factors associated with preterm birth (Trend, 2016).

Armogida, 2004 found the highest concentrations of HNP-1 and HBD-2; moderate amounts of HD6 and low amounts and HD5 and HBD-1 in colostrum. HNP-1, HD5 and HD6 were present in significantly higher amounts in colostrum compared with mature milk. There was a trend toward higher levels of HBD-2 in breast milk from women who delivered premature infants when compared with those who delivered at term. Trend recently found the most abundant proteins measured to be lactoferrin, sIgA, lysozyme and sCD-14. Defense peptides HBD-1 and HBD-2 were present in lower quantities. Median values of IL-10, IL-13, TNF-α, IFN-γ and HD5 were at the limit of detection, as these molecules were measurable in fewer than half of the breast milk samples. Introduction of suboptimal enteral formula diets coupled with parenteral nutrition, predispose to disease, while advancing amounts of mothers milk from birth and in particular colostrum protects against disease (Siggers, 2010).

Current Treatment of Preterm Infants

With increasing and earlier use of antenatal corticosteroids, assisted ventilation and surfactant, and changing attitudes towards intensive care, survival rates for very preterm births, especially those born before 28 week's gestation, has improved strikingly since the mid 1990's (Saigal, 2008).

A Cochrane analysis from 2014 concluded that supplementation of the milk diet with enteral probiotics reduced the risk of severe NEC as well as mortality (AlFaleh, 2014). Probiotics are known to have several important effects on a cellular level—NF_{K}B activation, upregulation of cytoprotective genes, prevention of apoptosis, generation of reactive oxygen species and expression of tight junctions (Patel, 2012). Several reports of probiotic associated sepsis have however raised concerns regarding routine clinical use of live bacteria in hosts, such as premature infants, who have immature epithelial-barrier defenses (Patel, 2012).

Antimicrobial Peptides

At birth, the immune system, especially the adaptive immunity, is immature, and characterized by low levels of immunoglobulins, naïve T cells and antigen presenting cells. The current hypothesis is that certain innate immune cells, which harbour effector molecules such as antimicrobial peptides and proteins, compensate to some degree for this impairment and play a major role in protecting against microbes in early life (Kai-Larsen, 2014). Antimicrobial peptides are already expressed when the foetus is in the womb, but the levels increase with gestational age. HD5 and HD6 transcripts have already appeared at 13 weeks of gestation (Mallow, 1996) and low levels of HD5 have been detected in the small intestine at 24 weeks of gestation (Salzman, 1998). Campeotto, 2010 found that hBD-2 can be detected at high level in the feces of full-term and preterm infants, independently of gestational age or mode of feeding. In conclusion, there is a big need for new treatments of infants born prematurely. There is a particular need for treatments that can facilitate the maturation of the mucosal defense in both the lungs and intestines simultaneously. There is a need for a treatment that can be administered through both oral, pulmonary and subcutaneous administration.

### SUMMARY

The inventor has surprisingly demonstrated that mammalian defensins have the ability to change the gut microbiota through an increase of microbial presence and abundance; to increase mucosal integrity through an increase in IL-22 concentration; to reduce the incidence of necrotizing enterocolitis; to reduce airway hyperresponsiveness (AHR) and increase airway compliance (Cdyn); particularly to improve lung function through a reduction of AHR and increase of airway compliance (Cdyn); to reduce lung inflammation; to reduce neutrophil-, eosinophil- and macrophage count in bronchio-alveolar-lavage-fluid (BALF) as well as rebalance the immune system through normalization of IFN-γ, TNF-α, IL-4, IL-5, IL-6, IL-9, IL-10 and IL-13 concentrations. The inventor has also demonstrated efficacy in reduction of histological inflammation in an asthma model. Particularly, the inventor has also demonstrated efficacy in reduction of histological inflammation in the lungs in an asthma model as well as in the intestines and liver in a model of graft versus host disease.

The data indicate that administration of mammalian defensins results in reduction of the major complications associated with preterm birth (IDC10; P07) and therefore are useful in treatment or prevention of sepsis (IDC10; P36, P37.5 and P52), necrotizing enterocolitis (IDC10; P77), short bowel syndrome (IDC10; K91.2), impaired neurodevelopment (IDC10; P52 and P91) and extrauterine growth restriction (IDC10; P92) in preterm infants.

Surprisingly it has been demonstrated in both a preventive and a therapeutic high fat diet mouse model that both α- and β-defensins have a strong influence on the composition of the microbiota but also that these effects are different from one defensin to another and that the combination of an α- and a β-defensin is yet again different from the individual effects.

It has further surprisingly been demonstrated in a mouse model of Dithizone/Klebsiella induced necrotizing enterocolitis that 3-defensins can prevent or dramatically reduce the incidence of necrotizing enterocolitis possibly facilitated through increased mucosal integrity by increase of IL-22 production.

It has further surprisingly been demonstrated in a mouse model of house dust mite allergy that a dosage of human beta-defensin 2 (hBD-2) whether administered orally or intranasally is capable of preventing the development of asthma and impaired lung function as defined by increased AHR, decreased Cdyn, inflammatory cell count in BALF and inflammatory cytokine production in lung tissue in a steroid-sensitive murine model, where mice are immunized by house dust mite (HDM)+Freund's adjuvant and challenged with HDM. Contrary to conventional asthma prophylaxis, where immune suppression is the target, prophylaxis with hBD-2 seems to rebalance the immune system and thus maintain a well-functioning immune response.

It has further surprisingly been demonstrated in a mouse model of house dust mite allergy that a dosage of human beta-defensin 2 (hBD-2) whether administered orally or intranasally is also capable of treating asthma and improve lung function by decreasing AHR, increasing Cdyn, reducing histological lung inflammation, reducing inflammatory cell count in BALF and inflammatory cytokine production in lung tissue in a steroid-sensitive murine model, where mice are immunized by house dust mite (HDM)+Freund's adjuvant and challenged with HDM.

Without hBD-2 treatment, animals developed asthma characterized by dramatically increased AHR, decreased Cdyn, inflammatory histological changes of the lung tissue, increased white blood cell count, in particular neutrophils, eosinophils and macrophages and increased concentration of inflammatory cytokines.

It has further surprisingly been demonstrated in human PBMC's, a marker of systemic toxicity, that the systemic toxicity of defensins varies. hBD-1, hBD-2 and HD5 were found not to be toxic in PBMC's. hBD-4 was found to be moderately toxic, whereas hBD-3 was found to be toxic. This is an important and surprising finding as Sheng, 2014 found hBD-3 not to be toxic, when tested in gut epithelial cell lines. Shengs finding was not surprising as the beta defensins are expressed on epithelial surfaces. The epithelial surfaces in the preterm infant e.g. the gut are however immature and leaky and orally administered defensins are thus likely to be absorbed and exert their effect systemically.

It has further surprisingly been demonstrated in a murine model of graft versus host disease, a model that shares a number of similarities to the preterm infant that prophylactic treatment with hBD-2 dramatically decreased mortality and weight loss; decreased the histology score of the small and large intestine as well as the liver all suggesting improved gut health and gut integrity. Treatment with hBD2 reduced the infiltration with CD45+leucocytes in gut epithelium/lamina propria and reduced intestinal T cell and myeloid cell infiltration. Prophylactic treatment with hBD2 also showed reduction of TNF-α and IL-6 and induction of IL-10. The hBD-2 treatment additionally showed a reduction of IL-1β from myeloid cells (FACS analysis of the spleen and reduced Th1 cytokine especially TNF-α and IFN-γ production.

Therefore in one aspect, there is provided a method of maturation/normalization of the intestinal microbiota, encompassing increased presence and abundance of key commensal bacteria e.g. bifidobacteriaceae, in a preterm infant, the method comprising administration of one or more mammalian antimicrobial peptides selected from the group consisting of defensins, cathelicidins, lactoferrins, lactoferricins and lysozymes to a preterm infant or a woman about to give birth to a preterm infant.

In other aspects there is provided a method for treatment and/or prevention of necrotizing enterocolitis, gut immaturity, dysmolality, increased gut permeability, sepsis, maturation/normalization of the mucosal defense as well as increased IL-22 production in the intestines in a preterm infant, the method comprising administration of at least one antimicrobial peptide selected from the group consisting of defensins, cathelicidins, lactoferrins, lactoferricins and lysozymes to a preterm infant or to a woman about to give birth to a preterm infant.

In other aspects there is provided a method of treatment and/or prevention of inflammatory diseases of the respiratory system (IDC10; P22 and P23) in a preterm infant, the method comprising administration of at least one antimicrobial peptide selected from the group consisting of defensins, cathelicidins, lactoferrins, lactoferricins and lysozymes to a preterm infant or to a woman about to give birth to a preterm infant.

In other aspects there is provided a method for treatment and/or prevention of a disease selected from the group consisting of sepsis, necrotizing enterocolitis, short bowel syndrome and extrauterine growth restriction in the preterm infant, the method comprising administration of at least one antimicrobial peptide selected from the group consisting of defensins, cathelicidins, lactoferrins, lactoferricins and lysozymes to a preterm infant or to a woman about to give birth to a preterm infant.

In other aspects there is provided a method for treatment and/or prevention of impaired neurodevelopment, white-matter injury, cerebral palsy, mental retardation and sensory impairments in the preterm infant, the method comprising administration of at least one antimicrobial peptide selected from the group consisting of defensins, cathelicidins, lactoferrins, lactoferricins and lysozymes to a preterm infant or to a woman about to give birth to a preterm infant.

Further is provided methods of improving gut health in a preterm infant, the method comprising administration of at least one antimicrobial peptide selected from the group consisting of defensins, cathelicidins, lactoferrins, lactoferricins and lysozymes to a preterm infant or to a woman about to give birth to a preterm infant.

Further is provided methods of reducing histological lung inflammation, perivascular and bronchiovascular inflammation, BALF inflammatory cell count, and/or inflammatory cytokine production in lung tissue in a preterm infant, the method comprising administration of at least one antimicrobial peptide selected from the group consisting of defensins, cathelicidins, lactoferrins, lactoferricins and lysozymes to a preterm infant or to a woman about to give birth to a preterm infant.

In another aspect there is provided a method for increasing pulmonary compliance, for reducing airway hyper responsiveness, and/or for increasing the peak expiratory flow in a preterm infant, said method comprising administration of at least one antimicrobial peptide selected from the group consisting of defensins, cathelicidins, lactoferrins, lactoferricins and lysozymes to a preterm infant or to a woman about to give birth to a preterm infant.

In another aspect there is provided a method for increasing lung function and pulmonary compliance, for reducing airway hyper responsiveness, and/or for increasing the peak expiratory flow in a preterm infant, said method comprising administration of at least one antimicrobial peptide selected from the group consisting of defensins, cathelicidins, lactoferrins, lactoferricins and lysozymes to a preterm infant or to a woman about to give birth to a preterm infant.

Further is provided a method for rebalancing the immune system through normalization of the tissue cytokine production and thus preventing a cytokine storm despite inflammatory cell migration into the bronchioalveolar fluid in a preterm infant, said method comprising administration of at least one antimicrobial peptide selected from the group consisting of defensins, cathelicidins, lactoferrins, lactoferricins and lysozymes to a preterm infant or to a woman about to give birth to a preterm infant.

In other aspects, the disclosure relates to an antimicrobial polypeptide selected from the group consisting of defensins, cathelicidins, lactoferrins, lactoferricins and lysozymes for use in a method of treatment according to any of the methods described herein.

In yet another aspect, the disclosure relates to the use of an antimicrobial polypeptide selected from the group consisting of defensins, cathelicidins, lactoferrins, lactoferricins and lysozymes for the manufacture of a medicament for the treatment of a disorder as defined herein.

### DESCRIPTION OF DRAWINGS

FIG. 1.

Schematic outline of the experimental setup for investigating the effects of mammalian defensins (HD5, hBD-2 and HD5+hBD-2) on the composition of the microbiota in a high fat diet murine model.

FIG. 2.

Schematic outline of the experimental set up for investigating the effects of prophylactic treatment with mammalian defensins in a murine steroid-sensitive model for prevention of asthma, where the mice are immunized by house dust mite (HDM)+Freund's adjuvant and challenged with HDM.

FIG. 3.

Schematic outline of the experimental set up for investigating the effects of treatment with mammalian defensins in a murine steroid-sensitive model for treatment of asthma, where the mice are immunized by house dust mite (HDM)+Freund's adjuvant and challenged with HDM.

FIG. 4.

Clustal W (2.1) multiple sequence alignment of A) human beta defensin 1-4 and B): HD5 and HD6

In the Clustal W alignments:
* indicates positions which have a single, fully conserved residue.
: indicates that one of the following ‘strong’ groups is fully conserved: S,T,A; N,E,Q,K; N,H,Q,K; N,D,E,Q; Q,H,R,K; M,I,L,V; M,I,L,F; H,Y; F,Y,W.
. indicates that one of the following ‘weaker’ groups is fully conserved: -C,S,A; A,T,V; S,A,G; S,T,N,K; S,T,P,A; S,G,N,D; S,N,D,E,Q,K; N,D,E,Q,H,K; N,E,Q,H,R,K; V,L,I,M; H,F,Y.

FIG. 5.

Unweighted and weighted unifrac analysis of microbial presence (describing the variety of bacteria present) and abundance (describing the amount of a given bacteria present as a percentage of all bacteria present) following prophylactic treatment with oral HD5, hBD-2 and HD5+hBD-2 (mix) in a murine high fat diet model. Untreated high fat diet (HF) and low fat diet (LF) are used as controls.

FIG. 6.

Genus analysis of microbial abundance following prophylactic treatment with oral HD5, hBD-2 and HD5+hBD-2 (mix) in a murine high fat diet model.

FIG. 7.

Abundance of Allobaculum in the small intestine following prophylactic treatment with oral HD5 and hBD-2 in a murine high fat diet model.

FIG. 8.

Abundance of Lactobacillaceae in colon following prophylactic treatment with oral hBD-2 in a murine high fat model.

FIG. 9.

Relative abundance of Barnesiella in colon following 4 and 10 weeks of prophylactic treatment with oral hBD-2 in a murine high fat diet model.

FIG. 10.

Unweighted unifrac analysis of microbial presence and abundance (see text to FIG. 5) following therapeutic treatment with HD5 or hBD-2 in a murine high fat diet model.

FIG. 11.

Relative abundance of Alloprevotella in colon following therapeutic intervention with oral HD5 and hBD-2 in a murine high fat diet model.

FIG. 12.

Relative abundance of Bifidobacteriaceae in the small intestine and colon following therapeutic intervention with HD5 or hBD-2 in a murine high fat diet model.

FIG. 13.

Clinical score (body weight loss, stool consistency and presence of blood per rectum) in a murine 14-week therapeutic SCID CD4+CD25+Tcell transfer colitis model showing effect of hBD-2, 1 mg/kg s.c. OD on par with 100 μg/mouse anti TNF-α s.c. twice weekly (Enbrel) and 0.3 mg/kg dexamethasone intraperitoneally OD.

FIG. 14.

Colon weight in a murine 14-week therapeutic SCID CD4+CD25+Tcell transfer colitis model showing effect of hBD-2, 1 mg/kg s.c. OD on par with 100 μg/mouse anti TNF-α s.c. twice weekly (Enbrel) and 0.3 mg/kg dexamethasone intraperitoneally OD.

FIG. 15

Myeloperoxidase activity in intestinal tissue as an expression of gut health in a murine 14-week therapeutic SCID CD4+CD25+ Tcell transfer model of colitis showing effect of 1 mg/kg s.c. hBD-2 on par with 100 μg/mouse anti TNF-α s.c. twice weekly (Enbrel) and 0.3 mg/kg dexamethasone intraperitoneally OD.

FIG. 16:

Airway hyper responsiveness in the murine House Dust Mite steroid-sensitive asthma model following prophylactic intranasal and oral administration of hBD-2 respectively.

FIG. 17:

Pulmonary compliance in the murine House Dust Mite steroid-sensitive asthma model following prophylactic intranasal and oral administration of hBD-2 respectively.

FIG. 18.

Cytokine concentrations of TNF-αin lung homogenate in the House Dust Mite murine steroid-sensitive asthma model following prophylactic intranasal (A) and oral (B) administration of hBD-2 respectively.

FIG. 19

Cytokine concentrations of IL-4 in lung homogenate in the House Dust Mite murine steroid-sensitive asthma model following prophylactic intranasal (A) and oral (B) administration of hBD-2 respectively.

FIG. 20

Cytokine concentrations of IL-5 in lung homogenate in the House Dust Mite murine steroid-sensitive asthma model following prophylactic intranasal (A) and oral (B) administration of hBD-2 respectively.

FIG. 21

Cytokine concentrations of IL-6 in lung homogenate in the House Dust Mite murine steroid-sensitive asthma model following prophylactic intranasal (A) and oral (B) administration of hBD-2 respectively.

FIG. 22

Cytokine concentrations of IL-9 in lung homogenate in the House Dust Mite murine steroid-sensitive asthma model following prophylactic intranasal (A) and oral (B) administration of hBD-2 respectively.

FIG. 23

Cytokine concentrations of IL-13 in lung homogenate in the House Dust Mite murine steroid-sensitive asthma model following prophylactic intranasal (A) and oral (B) administration of hBD-2 respectively.

FIG. 24

Cytokine concentrations of IL-33 in lung homogenate in the House Dust Mite murine steroid-sensitive asthma model following prophylactic intranasal (A) and oral (B) administration of hBD-2 respectively.

FIG. 25:

Airway hyper responsiveness in the House Dust Mite murine steroid-sensitive asthma model following therapeutic intranasal (A) and oral (B) administration of hBD-2 respectively. Saline is the non-challenged control. HDM/Vehicle is the House Dust Mite challenged control treated with vehicle.

“hBD-2 IN 1.2 mpk” is hBD-2 administered intranasally at 1.2 mg/kg. 5 mpk is 5 mg/kg.

FIG. 26:

Pulmonary compliance in the House Dust Mite murine steroid-sensitive asthma model following therapeutic intranasal (A) and oral (B) administration of hBD-2 respectively.

FIG. 27:

Total and differential cell count in BALF in the House Dust Mite murine steroid-sensitive asthma model following therapeutic intranasal administration of hBD-2

FIG. 28.

Cytokine concentrations of IFN-γ in lung homogenate in the House Dust Mite murine steroid-sensitive asthma model following therapeutic intranasal and oral administration of hBD-2 respectively. Data from the intranasal arm is shown on the left and data from the peroral arm is shown on the right.

FIG. 29

Cytokine concentrations of TNF-α in lung homogenate in the House Dust Mite murine steroid-sensitive asthma model following therapeutic intranasal and oral administration of hBD-2 respectively. Data from the intranasal arm is shown on the left and data from the peroral arm is shown on the right.

FIG. 30

Cytokine concentrations of IL-6 in lung homogenate in the House Dust Mite murine steroid-sensitive asthma model following therapeutic intranasal and oral administration of hBD-2 respectively.

FIG. 31

Cytokine concentrations of IL-9 in lung homogenate in the House Dust Mite murine steroid-sensitive asthma model following therapeutic intranasal and oral administration of hBD-2 respectively.

FIG. 32

Cytokine concentrations of IL-4 in lung homogenate in the House Dust Mite murine steroid-sensitive asthma model following therapeutic intranasal and oral administration of hBD-2 respectively. Data from the intranasal arm is shown on the left and data from the peroral arm is shown on the right.

FIG. 33

Cytokine concentrations IL-5 in lung homogenate in the House Dust Mite murine steroid-sensitive asthma model following therapeutic intranasal and oral administration of hBD-2 respectively.

FIG. 34

Cytokine concentrations of KC (IL-8) in lung homogenate in the House Dust Mite murine steroid-sensitive asthma model following therapeutic intranasal and oral administration of hBD-2 respectively.

FIG. 35

Cytokine concentrations of IL-10 in lung homogenate in the House Dust Mite murine steroid-sensitive asthma model following therapeutic intranasal and oral administration of hBD-2 respectively. Data from the intranasal arm is shown on the left and data from the peroral arm is shown on the right.

FIG. 36

Cytokine concentrations of IL-13 in lung homogenate in the House Dust Mite murine steroid-sensitive asthma model following therapeutic intranasal and oral administration of hBD-2 respectively.

FIG. 37.

Lung histology with H&E/PAS preparation in the House Dust Mite murine steroid-sensitive asthma model following therapeutic intranasal and oral administration of hBD-2 respectively. Upper left panel: untreated and unchallenged control. Upper right panel: untreated and HDM challenged control. Lower left panel: HDM challenged treated with hBD-2 PO. Lower right panel: HDM challenged treated with hBD-2 IN. 50× enlargement.

FIG. 38.

Lung inflammation severity in the House Dust Mite murine steroid-sensitive asthma model following therapeutic intranasal and oral administration of hBD-2 respectively.

FIG. 39.

Cell viability of PBMC cells following exposure for 24 hours to Alamar Blue and HD5, hBD-1, hBD-2, hBD-3 and hBD-4 at concentrations of 1, 10 and 40 μg/ml.

FIG. 40.

Histological scoring system for evaluation of severity of necrotizing enterocolitis in a murine model of Klebsiella/Dithizone induced necrotizing enterocolitis. >2 indicates significant gut inflammation.

FIG. 41.

A statistically significant reduction of the incidence of necrotizing enterocolitis is noted 16 hours post challenge in the mice that received one dose of 1.2 mg/kg hBD-2 one hour post challenge with Klebsiella/Dithizone.

FIG. 42.

A statistically significant gut mucosa protective increase of IL-22 is noted 9 hours post challenge in the group of mice that received one dose of 1.2 mg/kg hBD-2 one hour post challenge with Klebsiella/Dithizone.

FIG. 43.

A highly statistically significant reduction of mortality (p<0.0001) in 15 mice treated with oral hBD-2 for 10 days from the day of stem cell transplantation compared with 15 mice treated with PBS.

FIG. 44.

A statistically significant reduction of histology score of the small intestine, colon and liver in 10 mice treated with oral hBD-2 for 10 days from the day of stem cell transplantation.

FIG. 45.

Weight loss in % from baseline (a) and in gram (b) in mice treated with oral hBD-2 for 10 days from the day of stem cell transplantation.

FIG. 46.

Reduced CD45+ leucocyte cell migration into lamina propria of colon and small intestine in 10 mice treated with oral hBD-2 for 10 days from the day of stem cell transplantation.

FIG. 47 a-c.

Reduction of intestinal T cell and myeloid cell infiltration of the lamina propria of the colon and small intestine in 10 mice treated with oral hBD-2 for 10 days from the day of stem cell transplantation.

FIG. 48 a-c.

Cytokine concentrations of TNF-α (a), IL-6 (b) and IL-10 (c) in serum of 10 mice treated with oral hBD-2 for 10 days from the day of stem cell transplantation.

FIG. 49 a-c.

Reduced IL-1β production in myeloid cells in 10 mice treated with oral hBD-2 for 10 days from the day of stem cell transplantation.

FIG. 50.

Reduced proportion of neutrophils (a) and reduced Th 1 cytokine production—IFN-γ in CD4 T cells (b) and CD8 T cells (c) and CD69+CD4 T cells (d); TNF-α in CD4 T cells (e) and CD8 T cells (f) in 10 mice treated with oral hBD-2 for 10 days from the day of stem cell transplantation.

FIG. 51.

Increased inflammation, myeloid cell and leucocyte infiltration as well as increased tissue repair in colon in 10 mice treated with oral PBS for 10 days from the day of stem cell transplantation.

FIG. 52.

A statistically significant reduction of mortality (p=0.03) in 7 mice treated with oral hBD-2 for 10 days from the day of stem cell transplantation compared with 13 mice treated with cyclosporine and PBS respectively.

FIG. 53.

Weight loss in % from baseline in mice treated with oral hBD-2, cyclosporine or PBS for 10 days from the day of stem cell transplantation.

FIG. 54.

A significant reduction of mortality in 22 mice treated with oral hBD-2, oral HD5 or PBS respectively for 10 days from the day of stem cell transplantation.

FIG. 55.

Bacterial population control exerted by orally administered HD5 and hBD-2 measured as the distance from the intestinal lining to bacterial population (lysis zone) in μm in mice compared with mice on a low fat or western diet.

### DETAILED DESCRIPTION

### Definitions

The term “defensin” as used herein refers to polypeptides belonging to the defensin class of antimicrobial peptides. Defensins represent one of the dominant innate host defences that serve to maintain a healthy microbiome and ward off potential pathogens (Wehkamp et al. et al., 2002 and Salzman et al., 2007). Defensins are peptides possessing antimicrobial activity against Gram positive and negative bacteria, fungi and archaea as well as exerting anti-inflammatory activity.

Human defensins are small cationic peptides divided into α- and β-defensins based on the topology of their three intramolecular cysteine disulphide bonds. α-defensins can be further subdivided into those expressed in intracellularly in neutrophil granules (HNP1-4) and those expressed by Paneth cells in the crypts of the small intestine (HD5 and HD6 or DEFA5 and DEFA6). β-defensins (DEFBn) are mainly produced by epithelial cells in various tissues and organs including the skin, eye, middle ear, mouth, trachea, lungs, gastrointestinal tract, urogenital system, kidneys, vagina, liver, pancreas and mammary glands. Examples of defensins include human intestinal alpha defensin 5 (HD5; SEQ ID NO: 5); human intestinal alpha defensin 6 (HD6; SEQ ID NO: 6); human neutrophil peptide 1 (HNP-1; SEQ ID NO: 8); human neutrophil peptide 2 (HNP-2; SEQ ID NO:9); human neutrophil peptide 3 (HNP-3; SEQ ID NO: 10); human neutrophil peptide 4 (HNP-4; SEQ ID NO: 11) all belonging to the alfa defensin class; and also human beta defensin 1 (hBD-1; SEQ ID NO: 1); human beta defensin 2 (hBD-2; SEQ ID NO: 2); human beta defensin 3 (hBD-3; SEQ ID NO: 3); human beta defensin 4 (hBD-4; SEQ ID NO: 4); mouse beta defensin 3 (SEQ ID NO: 7); and truncated hBD-2 (SEQ ID NO: 16). Defensins are expressed as precursors and are processed by cleavage of the signal peptide and in some cases pro-peptides as well before secretion into the extracellular space. The best characterized members of the human β-defensin family are hBD-1-4. Some of the human defensins e.g. hBD-1 are produced constitutively, whereas others e.g. hBD-2, hBD-3 and hBD-4 are induced by pro-inflammatory cytokines or exogenous microbial products. The above-identified sequences represent the predicted mature bioactive defensins. It will be understood by one of skill in the art that processing may differ from cell to cell and that the resulting secreted mature peptide may differ by one or two C- or N-terminal amino acids from the predicted sequences and still retain bioactivity.

The term “Cathelicidin”, also known as LL-37, as used herein relates to a family of antimicrobial peptides found in lysosomes of macrophages and Granulocyte/polymorphonuclear leukocytes. Cathelicidins serve a critical role in mammalian innate immune defense against invasive bacterial infection. The cathelicidin family of peptides are classified as antimicrobial peptides (AMPs). The AMP family also includes the defensins. Whilst the defensins share common structural features, cathelicidin-related peptides are highly heterogeneous. Members of the cathelicidin family of antimicrobial polypeptides are characterized by a highly conserved region (cathelin domain) and a highly variable cathelicidin peptide domain. One example is human cathelicidin (LL-37, SEQ ID NO: 12).

The term “Lactoferrin”, also known as lactotransferrin, as used herein is related to a multifunctional protein of the transferrin family. Lactoferrin is a globular protein with a molecular mass of about 80 kDa that is widely represented in various secretory fluids, such as milk, saliva, tears, and nasal secretions. Lactoferrin is also present in secondary granules of neutrophil granulocytes and is secreted by acinar cells. Lactoferrin can be purified from milk or produced recombinantly. Human colostrum has the highest concentration, followed by human milk, then cow milk (150 mg/L). Lactoferrin is one of the components of the immune system of the body; it has antimicrobial activity (bacteriocidal, fungicidal) and is part of the innate defense. One example is human lactoferrin (SEQ ID NO: 13)

The term “Lactoferricin” as used herein relates to an amphipathic, cationic peptide with anti-microbial properties. It can be generated by pepsin-mediated digestion of lactoferrin. One example is lactoferricin-H (SEQ ID NO: 14)

The term “Lysozyme”, also known as muramidase or N-acetylmuramide glycanhydrolase, as used herein refers to an antimicrobial enzyme produced by animals and forms part of the innate immune system. Lysozyme is a glycoside hydrolase that catalyzes the hydrolysis of 1,4-beta-linkage between N-acetylmuramic acid and N-acetyl-D-glucosamine residue in peptidoglycan, which is the major component of the gram-positive bacterial cell wall. This hydrolysis in turn compromises the integrity of bacterial cell walls causing lysis of the bacteria. Lysozyme is abundant in secretions including tears, saliva, human milk, and mucus. It is also present in cytoplasmic granules of the macrophages and the polymorphonuclear neutrophils. One example is human lysozyme (SEQ ID NO: 15)

The term“identity” as used herein refers to the relatedness between two amino acid sequences or between two nucleotide sequences. The degree of identity between two amino acid sequences is determined using the Needleman-Wunsch algorithm (Needleman and Wunsch, 1970, J. Mol. Biol. 48: 443-453) as implemented in the Needle program of the EMBOSS package (Rice et al., 2000, http://emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the nobrief option) is used as the percent identity and is calculated as follows: (Identical Residues×100)/(Length of Alignment−Total Number of Gaps in Alignment).

The term “normal microbiota” is used herein to indicate a microbiota that is not dysbiotic. Normal microbiota is characterized by having large gene richness. Normal intestinal microbiota is characterized by comprising bacteria belonging to the genera Bacteriodetes, Faecalibacterium, Roseburia, Blautia, Ruminococcus, Coprococcus, Bifidobacterium, Methanobrevibacter, Lactobacillus, Coprococcus, Clostridium, Akkermansia, Eubacterium.

Normal lung microbiota is characterized by comprising bacteria belonging to the genera Bacteroidetes, Firmicutes, and Proteobacteria with the core microbiota consisting of Pseudomonas, Streptococcus, Prevotella, Fusobacteria, Veillonella, Haemophilus, Neisseria and Porphyromonas

The term “improving intestinal health”, as used herein, refers to an increase in the mucosal proportion of the gut wall, increase in the height of the intestinal villi, increase in the density of intestinal goblet cells, increase in the digestive brush border enzyme activity and lower intestinal myeloperoxidase activity

The term “white-matter injury”, as used herein, is characterized by focal cystic periventricular leukomalacia, diffuse necrosis or both.

The term “sensory impairments”, as used herein, refers to conditions such as visual and auditory deficits.

The terms “treatment” and “treating” as used herein refer to the management and care of a patient for the purpose of combating a condition, disease or disorder. The term is intended to include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” is to be understood to refer to the management and care of a patient for the purpose of hindering, reducing the active compounds to prevent or reduce the risk of the onset of symptoms or complications. The patient to be treated is preferably a mammalian, in particular a human being. The term “patient” as used herein refers to a prematurely born infant or to a woman who is about to give birth prematurely (perterm labor).

The term “preterm infant”, as used herein, refers to an infant who has been born before gestational age of 37 weeks, such before gestational age of 35 week, for example 30 weeks, such as 25 weeks. The term includes VLBW (very low birth weight) infants with a birth weight below 1,500 g and ELBW (extremely low birth weight) infants with a birth weight below 1,000 g.

The term “woman about to give birth to a premature infant”, as used herein, refers to a pregnant woman who is in labor and about to give birth to an infant who has a gestational age of 37 or less, such a gestational age of 35 week or less, for example 30 weeks or less, such as 25 weeks or less. The woman about to give birth to a premature infant may also be a pregnant woman who has been diagnosed with a particular disorder such as an inflammatory disorder of the lungs or the intestines, suffers from particular symptoms indicative of a disorder, such as an inflammatory disorder of the lungs or the intestines. Such a pregnant woman is considered to be at risk of giving premature birth.

Mammalian Alfa Defensins, Mammalian Beta Defensins, Mammalian Cathelicidins, Mammalian Lactoferrins/Lactoferricins and Mammalian Lysozymes.

This disclosure relates to uses of mammalian alfa defensins and/or beta defensins and/or cathelicidins and/or lactoferrins and/or lactoferricins and/or lysozymes, such as human alfa and beta defensins, more preferably Hominidae, in the treatment or prophylaxis of sepsis, respiratory illness, necrotizing enterocolitis, acute and prolonged diarrhea, short bowel syndrome, impaired neurodevelopment and extrauterine growth restriction in the preterm infant or in a mother about to give birth prematurely.

In one embodiment, the antimicrobial peptides are selected from the group consisting of α-defensins, β-defensins, cathelicidins, lactoferrins, lactoferricins and lysozymes.

In one embodiment, the mammalian alfa or beta defensins are selected from the group consisting of HNP-1, HNP-2, HNP-3, HNP-4, HD5, HD6, hBD-1, hBD-2, hBD-3 and hBD-4. In another embodiment, the mammalian alfa or beta defensins are selected from the group consisting of HD5, HD6, hBD-1, hBD-2, and hBD-4. These peptides are all expressed in the intestinal mucosa and can collectively be called intestinal defensins as opposed to the HNPs that are not expressed on the surface. In a preferred embodiment, the mammalian alfa or beta defensins are selected from the group consisting of HD5 and hBD-2.

In an embodiment, the mammalian alfa and/or beta defensins have a degree of identity of at least 80%, preferably at least 85%, more preferably at least 90%, and most preferably at least 95% to any of the amino acid sequences of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10 and SEQ ID NO: 11. In another embodiment, a defensin differs from one of the SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10 and SEQ ID NO: 11by less than 10 amino acids, such as less than 8, for example less than 5, such as less than 4, for example less than 3, such as less than 2 amino acids. In another embodiment, the defensin is a fragment of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10 and SEQ ID NO: 11 having no more than 10 amino acids, such as less than 8, for example less than 5, such as less than 4, for example less than 3, such as less than 2 amino acids deleted from the N- and/or C-terminus of the sequence.

In a preferred embodiment, the human alfa defensins consist of (alfa defensin 5 (SEQ ID NO: 5) and/or alfa defensin 6 (SEQ ID NO: 6). In a preferred embodiment, the mammalian beta defensins consist of human beta defensin 1 (SEQ ID NO: 1), human beta defensin 2 (SEQ ID NO: 2), human beta defensin 3 (SEQ ID NO: 3) and/or human beta defensin 4 (SEQ ID NO: 4).

In a preferred embodiment, a human alfa defensin has a degree of identity of at least 80%, preferably at least 85%, more preferably at least 90%, and most preferably at least 95% to the amino acid sequence of SEQ ID NO: 5. In a preferred embodiment, the human mammalian alfa defensins consist of alfa defensin 5 (SEQ ID NO: 5).

In a preferred embodiment, the human beta defensin has a degree of identity of at least 80%, preferably at least 85%, more preferably at least 90%, and most preferably at least 95% to the amino acid sequence of SEQ ID NO: 2. In a preferred embodiment, the human beta defensins consists of human beta defensin 2 (SEQ ID NO: 2).

In yet another embodiment, the mammalian alfa defensins comprise of human alfa defensins and/or mouse and/or pig alfa defensins, and functionally equivalent variants thereof. Preferably, the mammalian alfa defensin consist of human alfa defensin 5, human alfa defensin 6 and functionally equivalent variants thereof. More preferably, the mammalian alfa defensins consist of human alfa defensin 5, and functionally equivalent variants or orthologues thereof.

In yet a further embodiment, the mammalian beta defensins consist of human beta defensins and/or mouse and/or pig beta defensins, and functionally equivalent variants thereof. Preferably, the mammalian beta defensins consist of human beta defensin 1, human beta defensin 2, human beta defensin 3, human beta defensin 4, and functionally equivalent variants thereof. More preferably, the mammalian beta defensins consist of human beta defensin 2, and functionally equivalent variants or orthologues thereof.

A “functionally equivalent variant” of a mammalian (e.g. human) alfa or beta defensin is a modified mammalian alfa or beta defensin exhibiting approximatively the same effect on microbiota in the lung or the intestine or the skin as the parent mammalian alfa and/or beta defensins. A functionally equivalent variant of a mammalian defensin may comprise 1-5 amino acid modifications, preferably 1-4 amino acid modifications, more preferably 1-3 amino acid modifications, most preferably 1-2 amino acid modification(s), and in particular one amino acid modification, as compared to the mammalian defensin amino acid sequence (e.g. any of SEQ ID NOs: 1-6 and SEQ ID NOs: 8-11). Preferably, for beta mammalian defensins, compared to human beta defensin 2, having SEQ ID NO: 2 and for alpha defensins compared to HD5 (SEQ ID NO: 5).

The term “modification” means herein any chemical modification of a mammalian (e.g. human) defensin. The modification(s) can be substitution(s), deletion(s) and/or insertions(s) of the amino acid(s) as well as replacement(s) of amino acid side chain(s); or use of unnatural amino acids with similar characteristics in the amino acid sequence. In particular the modification(s) can be amidations, such as amidation of the C-terminus. Preferably, amino acid modifications are of a minor nature, that is conservative amino acid substitutions or insertions that do not significantly affect the folding and/or activity of the polypeptide; single deletions; small amino- or carboxyl-terminal extensions; or a small extension that facilitates purification by changing net charge or another function, such as a poly-histidine tag, an antigenic epitope or a binding domain. In one embodiment the small extension, such as a poly-histidine tag, an antigenic epitope or a binding domain is attached to the mammalian alfa or beta defensin through a small linker peptide of up to about 20-25 residues and said linker may contain a restriction enzyme cleavage site.

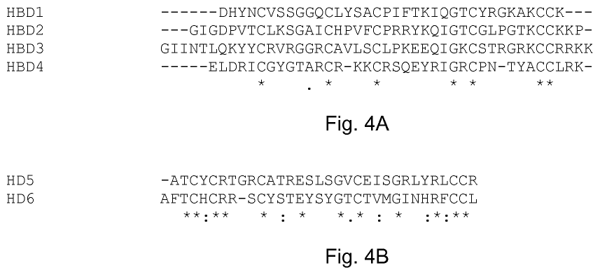

The Clustal W alignments in FIG. 4 can be used to predict which amino acid residues can be substituted without substantially affecting the biological activity of the protein. The sequences were aligned using Clustal W 2.1 (http://www.geno,me.jp/tools/clustalw/) and the following settings: Gap Open Penalty:10, Gap Extension Penalty: 0.05, Weight Transition: NO, Hydrophilic Residues for Proteins: GPSNDQE, Hydrophilic Gaps: YES, Weight Matrix: BLOSUM (for PROTEIN). Substitutions within the following group (Clustal W, ‘strong’ conservation group) are to be regarded as conservative substitutions:

-S,T,A; N,E,Q,K; N,H,Q,K; N,D,E,Q; Q,H,R,K; M,I,L,V; M,I,L,F; H,Y; F,Y,W. Substitutions within the following group (Clustal W, ‘weak’ conservation group) are to be regarded as semi-conservative substitutions: —C,S,A; A,T,V; S,A,G; S,T,N,K; S,T,P,A; S,G,N,D; S,N,D,E,Q,K; N,D,E,Q,H,K; N,E,Q,H,R,K; V,L,I,M; H,F,Y.

Examples of conservative substitutions are substitutions made within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by Neurath and Hill (1979). The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Essential amino acids in a mammalian alfa and/or beta defensin can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., activity against an airway hyper responsiveness or suppression of cytokines e.g. TNF-alpha activity) to identify amino acid residues that are critical to the activity of the molecule. See also, Hilton et al., 1996, J. Biol. Chem. 271: 4699-4708. The identities of essential amino acids can also be inferred from analysis of identities with polypeptides which are related to mammalian alfa and/or beta defensins (see Clustal W alignment in FIG. 4).

Single or multiple amino acid substitutions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30:10832-10837; U.S. Pat. No. 5,223,409; WO 92/06204), and region-directed mutagenesis (Derbyshire et al., 1986, Gene 46:145; Ner et al., 1988, DNA 7:127). When the result of a given substitution cannot be predicted with certainty, the derivatives may be readily assayed according to the methods described herein to determine the presence or absence of biological activity.

In one embodiment, the methods of treatment comprise administration of an effective amount of at least one mammalian α-defensin to a preterm infant or to a woman about to give birth to a preterm infant (preterm labor). In other embodiments, the provided methods comprise administration of an effective amount of at least one mammalian β-defensin to a preterm infant or to a woman about to give birth to a preterm infant. In a further embodiment, the provided methods comprise administration of an effective amount of at least one mammalian α-defensin and at least one mammalian β-defensin to a preterm infant or to a woman about to give birth to a preterm infant. A preferred embodiment provides administration of mammalian alfa defensin HD5 and/or mammalian beta defensin hBD-2.

Long-Acting Defensins, Cathelicidins, Lactoferrins/Lactoferricins and Lysozymes

The half-life of a mammalian α- or β-defensin, cathelicidin, lactoferrin/lactoferricin or lysozyme may be extended by fusing or conjugating the α- or β-defensin, cathelicidin, lactoferrin/lactoferricin or lysozyme with another moiety i.e. constructing a long acting biologically active α- or β-defensin, cathelicidin, lactoferrin/lactoferricin or lysozyme linked to a pharmaceutically acceptable molecule providing an in vivo plasma half-life of the α- or β-defensin, cathelicidin, lactoferrin/lactoferricin or lysozyme, which is increased substantially compared to the in vivo plasma half-life of the non-conjugated α- or β-defensin, cathelicidin, lactoferrin/lactoferricin or lysozyme administered in the same manner.

In one embodiment, the α- or β-defensin, cathelicidin, lactoferrin, lactoferricin or lysozyme further comprises at least one additional moiety selected from the group consisting of a cell penetrating peptide (CPP), an Albumin Binding Moiety (ABM), a detectable moiety (Z), and a half-life extending peptide.

A long acting biologically active α- or β-defensin, cathelicidin, lactoferrin/lactoferricin or lysozyme comprising a mammal α-defensin or analog thereof or a mammal β-defensin or analog thereof or a mammal cathelicidin or analog thereof or a mammal lactoferrin or analog thereof or a mammal lactoferricin or analog thereof or a mammal lysozyme or analog thereof linked to a pharmaceutically acceptable molecule selected from the group consisting of a molecule having binding to a neonatal Fc receptor (FcRn), transferrin, albumin (HAS), XTEN® or PEG, a homo-amino acid polymer (HAP), a proline-alanine-serine polymer (PAS), or an elastin-like peptide (ELP), hyaluronic acid, a negatively charged highly siasylated peptide such as the carboxy-terminal peptide (CTP) of chorionic gonadotropin (CG) 6-chain, human IgG, and CH3(CH2)ₙCO-wherein n is 8 to 22.

The α- or β-defensin, cathelicidin, lactoferrin/lactoferricin or lysozyme analog may also be of non-mammalian origin, and may be a peptide, a polypeptide or a protein.

The α- or β-defensin, cathelicidin, lactoferrin/lactoferricin or lysozyme analog may be linked to the pharmaceutically acceptable molecule in various ways as described in the prior art literature, such as without limitation chemical coupling through a bifunctional linker, gene technologically by coupling the N-terminal or C-terminal of the defensin, cathelicidin, lactoferrin/lactoferricin or lysozyme, such as α-defensin or β-defensin, to the pharmaceutically acceptable molecule, such as albumin or an albumin analog. In particular, the N-terminal of albumin or an albumin analogue, e.g. human albumin, can be coupled to the C-terminal of an α-defensin or β-defensin, cathelicidin, lactoferrin/lactofferricin or lysozyme, or the N-terminal of an α- or β-defensin, cathelicidin, lactoferrin/lactoferricin or lysozyme; or the C-terminal of albumin, e.g. human albumin, can be coupled to the C-terminal of an α-defensin or β-defensin, cathelicidin, lactoferrin/lactoferricin or lysozyme, or the N-terminal of α- or β-defensin, cathelicidin, lactoferrin/lactoferricin or lysozyme. A linker sequence can be inserted between the albumin and the α- or β-defensin, cathelicidin, lactoferrin/lactoferricin or lysozyme chain.

The α- or β-defensin, cathelicidin, lactoferrin/lactoferricin or lysozyme analog may be linked to the pharmaceutically acceptable molecule through a stable linker or a more labile linker. Several linkers are known in the art, including bifunctional PEG molecules (e.g. see Paige et. al Pharmaceutical Research, vol. 12, no. 12, 1995), hydrolysable linkers (Shechter et al. Bioconjugate Chem. 2005, 16: 913-920 and International Journal of Peptide Research and Therapeutics, Vol. 13, Nos. 1-2, June 2007 and WO2009095479), PDPH and EMCH see e.g. in WO2010092135. In the special case where chemical conjugation (linking of two or more molecules) of the α- or β-defensin, cathelidin, lactoferrin/lactoferricin or lysozyme agonist, to the pharmaceutically acceptable molecule, strongly reduce the functional α- or β-defensin, cathelicidin, lactoferrin/lactoferricin or lysozyme activity, it may be preferable to use a more labile linker that can release the functional α- or β-defensin, cathelicidin, lactoferrin/lactoferricin or lysozyme agonist.

Half-life extension may also be accomplished through acylation of the peptide backbone with a spacer e.g. γ-L-glutamyl spacer and a C-18 fatty di-acid chain to Lysine. The fatty di-acid site chain and the spacer mediate a strong but reversible binding to albumin, slowing release from the injection site and reducing renal clearance.

Methods and Uses

Human defensin 5, human beta defensin 2 and human defensin 5 plus human beta defensin 2 is found to be able to increase the microbial presence and abundance in the intestines; thus showing potent activity as a medicament for maturing or normalizing the intestinal microbiota as well as preventing or treating necrotizing enterocolitis, short bowel syndrome, sepsis, respiratory illness, neurodevelopment impairment and extrauterine growth restriction in a preterm infant.

Human beta defensin 2 is found to be able to decrease the incidence of necrotizing enterocolitis and prevent weight loss through a normalized gut function—increase mucosal proportion of the intestinal wall, increase the height of the intestinal villi and increase the density of goblet cells, increase the digestive brush border enzyme activity, lower the intestinal myeloperoxidase activity and increased IL-22 production; thus showing potent activity as a medicament for prevention or treatment of necrotizing enterocolitis, short bowel syndrome, sepsis, neurodevelopmental impairment and extrauterine growth restriction.

Human beta defensin 2 has been shown in a therapeutic animal model of severe intestinal inflammation and dysbiosis (CD4+CD25+ T-cell transfer), that β-defensins whether administered orally or subcutaneously mitigate weight loss, improve intestinal health and importantly decreases the disease activity index on par with strong immunosuppressants such as prednisolone/dexamethasone and cyclosporine as well as anti TNF-α, all routinely used in treatment of colitis such as Crohn's Disease and Ulcerative Coloitis in adults thus showing potent activity as a prophylactic or therapeutic treatment of preterm infants with necrotizing enterocolitis.

It has been demonstrated that β-defensins can normalize gut health and function through a decrease of myeloperoxidase activity, thus presenting a method for improving gut health in preterm infants.

Human beta defensin 2 is found to be able to prevent an increase of airway hyper responsiveness; to prevent a decrease of pulmonary compliance; to prevent lung inflammation; to prevent neutrophil-, eosinophil- and macrophage migration into BALF as well as normalize TNF-α, IL-4, IL-5, IL-6, IL-9 and IL-13 concentrations in lung tissue homogenate; thus showing potent activity as a medicament for prevention of respiratory illness and inflammatory conditions of the lungs, such as pneumonia and sepsis in a preterm infant.

Particularly, human beta defensin 2 and human defensin 5 have been demonstrated in a murine model of graft versus host disease to dramatically decreased mortality and weight loss on par or better than the standard treatment of graft versus host disease with cyclosporine. It has further been demonstrated that the defensins decreased the histology score of the small and large intestine as well as the liver all suggesting improved gut health and gut integrity. Treatment with defensins reduced the infiltration with CD45+ leucocytes as well as intestinal T cell and myeloid cell infiltration in gut epithelium/lamina propria. Prophylactic treatment with defensins also showed reduction of TNF-α and IL-6 and induction of IL-10. The defensins prophylactic treatment additionally showed a reduction of IL-1β from myeloid cells (FACS analysis of the spleen) and reduced Th1 cytokine especially TNF-α and IFN-γ production.

Human beta defensin 2 was found to be able to improve lung function through prevention of an increase of airway hyper responsiveness; to prevent a decrease of pulmonary compliance; to prevent lung inflammation; to prevent neutrophil-, eosinophil- and macrophage migration into BALF as well as normalize TNF-α, IL-4, IL-5, IL-6, IL-9 and IL-13 concentrations in lung tissue homogenate; thus showing potent activity as a medicament for prevention of respiratory illness and inflammatory conditions of the lungs, such as pneumonia and sepsis in a preterm infant.

Human beta defensin 2 is found to be able to reduce airway hyper responsiveness; increase pulmonary compliance; reduce lung inflammation; reduce BALF neutrophil-, eosinophil- and macrophage count as well as normalize IFN-γ, TNF-α, IL-4, IL-5, IL-6, IL-9, IL-10 and IL-13 concentrations in lung homogenate; thus showing potent activity as a medicament for treatment of inflammatory conditions of the lungs, such as pneumonia and sepsis in a preterm infant.

Human beta defensin 2 is found to be able to rebalance the immune system normalizing tissue cytokine production of IFN-γ, TNF-α, IL-4, IL-5, IL-6, IL-9, IL-10 and IL-13.

Surprisingly it has been found that both parenteral but also oral administration of defensins is effective to prevent and treat inflammatory conditions of the lung. This is unexpected, as it is known that hBD-2 is not absorbed from the gut. An advantage of this observation is that severely ill preterm infants on assisted ventilation can be treated by oral administration of the defensins.

Human beta defensin 2 has been found to mature and normalize the intestinal microbiota, prevent or treat inflammation of the gut and/or lung, sepsis, respiratory illness, neurodevelopmental impairment and extra uterine growth restriction in the preterm infant.

Therefore in one aspect the disclosure relates to methods for treatment of a preterm infant or a mother about to give birth to a preterm infant by administration of at least one antimicrobial peptide selected from the group consisting of defensins, cathelicidins, lactoferrins, lactoferricins and lysozymes to said preterm infant or to a woman about to give birth to a preterm infant. In one embodiment, the administration is oral, buccal, sublingual, rectal, vaginal, intratracheal, intrapulmonary, intranasal, intracranial, subcutaneous, intravenous, dermal or transdermal. Preferably the administration is oral. Oral and parenteral administration is advantageous for preterm infants with compromised breathing or preterm infants undergoing medical ventilation.

In another aspect there is provided methods for prevention or treatment of necrotizing enterocolitis, short bowel syndrome, sepsis, respiratory illness, impaired neurodevelopment and extrauterine growth restriction by administering an effective amount of a mammalian antimicrobial peptide from the group consisting of defensins, cathelicidins, lactoferrins/lactoferricins and lysozymes to a subject in need of such treatment. These conditions can be treated by oral or parenteral administration. Preferably, the administration is oral.

The provided methods can mature or normalize gut microbiota in a preterm infant by increasing the presence and abundance of key commensal bacteria.

The provided methods can improve intestinal health, normalize gut function and increase food uptake in a preterm infant through increasing the mucosal proportion of the gut wall, increasing the height of the intestinal villi and increasing the density of intestinal goblet cells, increasing the digestive brush border enzyme activity and lower intestinal myeloperoxidase activity.

The provided methods can treat or prevent respiratory illness, lung inflammation, respiratory tract infection, respiratory failure, pneumonia and sepsis in a preterm infant by reducing migration of white blood cells e.g. neutrophils, eosinophils and macrophages in BALF.

The provided methods can be used to prevent or treat respiratory distress syndrome. Infant respiratory distress syndrome (IRDS), also called neonatal respiratory distress syndrome (NRDS), respiratory distress syndrome of newborn, or increasingly surfactant deficiency disorder (SDD), and previously called hyaline membrane disease (HMD), is a syndrome in premature infants caused by developmental insufficiency of pulmonary surfactant production and structural immaturity in the lungs.

The provided methods can also be used to prevent or treat bronchopulmonary dysplasia. Bronchopulmonary dysplasia (BPD; formerly chronic lung disease of infancy) is a chronic lung disease in which premature infants, usually those who were treated with supplemental oxygen, require long-term oxygen. It is more common in infants with low birth weight and those who receive prolonged mechanical ventilation to treat respiratory distress syndrome (RDS). It results in significant morbidity and mortality.

The methods may also normalize cytokine production of e.g. IFN-γ, TNF-α, IL-113, IL-4, IL-5, IL-6, IL-8, IL-9, IL-10, IL-13 and IL-33 in lung tissue homogenate from a preterm infant affected by one of the said conditions as described herein and thus prevent a cytokine storm despite inflammatory cell migration into BALF.

The provided methods can treat or prevent lung and/or gut inflammation by changing bacterial phenotypes through a change at the transcriptional level as well as structure and composition of the lung and/or gut bacterial flora or the lung and/or gut metabolome of a subject affected by one of the said conditions as described herein.

Without being bound by theory the effects observed using oral administration may be ascribed to a change in the gut microflora and gut metabolome that may have an effect on the lungs through the so-called gut-lung axis. Chronic lung disorders such as asthma, COPD and cystic fibrosis all exhibit a component of intestinal disease manifestation indicating that there is a vital cross talk between these two mucosal sites of the human body and a variety of respiratory diseases have been associated with a dysbiosis not only of the airway microbiota but also the intestinal microbiota (Marsland et al, 2015). Caesarian birth reduces the diversity and alters the composition of the intestinal microbiota early in life and is at the same time linked to a predisposition towards asthma during childhood (Jakobsson et al, 2014).

Commensal microbes calibrate innate and adaptive immune responses and impact activation thresholds for pathogenic stimulations, in large part by producing small molecules that mediate host-microbial interactions (Donia and Fishback, 2015). While the epithelial barrier ensures that microorganisms are largely confined to the gut, microbial metabolites can penetrate the epithelial barrier, allowing them to enter and accumulate in the host circulatory system where they are sensed by immune cells (Dorrestein, 2014). Trompette, 2013 demonstrated in mice that fermentable fibers in the diet changed the composition not only of the gut but also the lung microbiota in particular the ratio of Firmicutes to Bacteriodetes, the latter leading to increased local and systemic levels of Short Chain Fatty Acids, which in turn influenced Dendritic Cell hematopoiesis and functionality thus shaping the immunological environment in the lung and influencing the severity of allergic inflammation. Schirmer et al (2016) further demonstrated in the Human Functional Genomics Project that inter-individual variation in cytokine response is linked to specific microbial organisms as well as microbial functions. The majority of detected associations were both cytokine and stimulus specific, suggesting that the immune system recognizes and interacts with microbial organisms and products with high specificity and that these microbial factors are associated with a particular immunological phenotype. TNF-α and IFN-γ production capacity appeared to be more strongly influenced by the microbiome, whereas other cytokines such as IL-1β, IL-6 and Th17 derived IL-17 and IL-22 exhibited fewer, but more specific, associations with the gut microbiota.

Further provided are methods for treatment of necrotizing enterocolitis, short bowel syndrome and extrauterine growth restriction in a preterm infant by administering an effective amount of an α- and/or a β-defensin and/or a cathelicidin and/or a lactoferrin/lactoferricin and/or a lysozyme to a preterm infant or a woman about to give birth to a preterm infant.

Further provided are methods for prevention of necrotizing enterocolitis, sepsis and impaired neurodevelopment in a preterm infant by administering an effective amount of an α- and/or a β-defensin.

The methods of treatment described herein can be performed by administration of a composition comprising at least one mammalian α- and/or β-defensin in combination with either a mammalian lactoferrin/lactoferricin, a mammalian lysozyme, a mammalian cathelicidin, surfactant, prebiotics, probiotics, glucocorticoids, antibiotics, immunosuppressants, GLP-2 or GLP-2 analogs or a combination of these. The defensins can be administered separately or together with one or more of these therapies. The defensins can also be administered together with other medicaments which can be used in the preterm infant or administered to a mother in preterm labor. Importantly, the disclosed methods can be used for treatment, prevention or normalization of a dysbiotic microbiota/metabolome in the gut and/or lung of a preterm infant and/or an infant who's mother during pregnancy has undertaken an antibiotic treatment or immunosuppressive therapy, or another treatment that has negative effects on the lung or gut microbiota.

Normalizing the gut and/or lung microbiota may also involve changing the metabolome to one that produces relatively more butyrate and relatively less acetate.

In Vitro Synthesis

The mammalian antimicrobial peptides including mammalian alfa defensins, mammalian beta defensins, mammalian cathelicidins, mammalian lactoferrins/lactoferricins and mammalian lysozymes of the invention may be prepared by in vitro synthesis, using conventional methods as known in the art. Various commercial synthetic apparatuses are available, for example automated synthesizers by Applied Biosystems Inc., Beckman, etc. By using synthesizers, naturally occurring amino acids may be substituted with unnatural amino acids, particularly D-isomers (or D-forms) e.g. D-alanine and D-isoleucine, diastereoisomers, side chains having different lengths or functionalities, and the like. The particular sequence and the manner of preparation will be determined by convenience, economics, purity required, and the like. Chemical linking may be provided to various peptides or proteins comprising convenient functionalities for bonding, such as amino groups for amide or substituted amine formation, e.g. reductive amination, thiol groups for thioether or disulphide formation, carboxyl groups for amide formation, and the like. If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

Mammalian antimicrobial peptides including mammalian alfa defensins, mammalian beta defensins, mammalian cathelicidins, mammalian lactoferrins/lactoferricins or mammalian lysozymes or functional equivalents thereof, may also be isolated and purified in accordance with conventional methods of recombinant synthesis. Recombinant synthesis may be performed using appropriate expression vectors and a eukaryotic or prokaryotic expression system. A solution may be prepared of the expression host and the media and the defensins present purified using HPLC, exclusion chromatography, gel electrophoresis, affinity chromatography, or other purification technique. Methods for recombinant expression of human beta defensin-2 in E. coli are disclosed in WO 2010/007166 (Novozymes).

The mammalian antimicrobial peptides including mammalian alfa defensins, mammalian beta defensins, mammalian cathelicidins, mammalian lactoferrins/lactoferricins or mammalian lysozymes may also be induced by administration of the corresponding mRNA to a preterm infant or to a woman about to give birth to a preterm infant.

Dosages

A mammalian alfa defensin, a mammalian beta defensin, a mammalian cathelicidin, a mammalian lactoferrin/lactoferricin, a mammalian lysozyme, such as a human alfa defensin, a human beta defensin, a human cathelicidin, a human lactoferrin/lactoferricin, a human lysozyme are preferably employed in pharmaceutical compositions in an amount which is effective to prevent or treat necrotizing enterocolitis, short bowel syndrome, sepsis, respiratory illness, impaired neurodevelopment and extrauterine growth restriction in the preterm infant or in the mother about to give preterm birth preferably with acceptable toxicity to the patient. A mammalian alfa defensin and a mammalian beta defensin, a mammalian cathelicidin, a mammalian lactoferrin/lactoferricin, a mammalian lysozyme, such as a human alfa defensin, a human beta defensin, a human cathelicidin, a human lactoferrin/lactoferricin, a human lysozyme are also preferably employed in pharmaceutical compositions in an amount which is effective to maintain a normal microbiota composition in the lung and/or the intestine or to treat or normalize a dysbiotic microbiota in the lung and/or the intestine, preferably with acceptable toxicity to the patient in need of the treatment.

For such treatments, the appropriate dosage will, of course, vary depending upon, for example, the chemical nature and the pharmacokinetic data of a compound used, the individual host, the mode of administration and the nature and severity of the conditions being treated.

However, in general, for satisfactory results in mammals, for example humans, an indicated daily dosage of a human alfa defensin is preferably from about 0.1 mg HD5/kg body weight to about 10 mg HD5/kg body weight, more preferably from about 0.5 mg HD5/kg body weight to about 10 mg HD5/kg body weight; such as 1 mg HD5/kg body weight to 10 mg HD5/kg body weight, more preferably from about 1.2 mg HD5/kg body weight to about 10 mg HD5/kg body weight, preferably from about 1.2 mg HD5/kg body weight to about 5 mg HD5/kg body weight, even more preferably 1.2 mg HD5/kg body weight, for example, administered in divided doses up to one, two or three times a day.

In one embodiment an indicated daily dosage of a human beta defensin is preferably from about 0.1 mg hBD-2/kg body weight to about 10 mg hBD-2/kg body weight, more preferably from about 0.5 mg hBD-2/kg body weight to about 10 mg hBD-2/kg body weight; such as 1 mg hBD-2/kg body weight to 10 mg hBD-2/kg body weight, more preferably from about 1.2 mg hBD-2/kg body weight to about 10 mg hBD-2/kg body weight, preferably from about 1.2 mg hBD-2/kg body weight to about 5 mg hBD-2/kg body weight, even more preferably 1.2 mg hBD-2/kg body weight, for example, administered in divided doses up to one, two or three times a day.

When two different defensins are administered in one dosage, the dosage may comprise equal or approximately equal amounts of the two defensins determined on a weight basis or on a molar basis. The ratio may also differ so that the ratio of alpha defensin to beta-defensin varies from 10:1 to 1:10, such as 5:1 to 1:5, for example 2:1 to 1:2 determined on a weight or molar basis.

The compounds of preferred embodiments can be administered to mammals, for example humans, by similar modes of administration at similar dosages than conventionally used.

In one embodiment, methods are provided as described herein, wherein the daily dosage is between 0.1 and 10 mg defensin/kg, such as between 0.5 and 5 mg defensin/kg, such as between 1 and 2 mg defensin/kg, such as 1.2 mg defensin/kg per day.

In one embodiment, methods are provided as described herein, wherein the daily dosage is between 0.1 and 10 mg cathelicidin/kg, such as between 0.5 and 5 mg cathelicidin/kg, such as between 1 and 2 mg cathelicidin/kg, such as 1.2 mg cathelicidin/kg per day.

In one embodiment, methods are provided as described herein, wherein the daily dosage is between 1 and 1000 mg lactoferrin/kg, such as between 5 and 500 mg lactoferrin/kg, such as between 50 and 200 mg lactoferrin/kg such as 100 mg/kg lactoferrin/kg per day.

In one embodiment, methods are provided as described herein, wherein the daily dosage is between 1 and 1000 mg lysozyme/kg, such as between 5 and 500 mg lysozyme/kg, such as between 50 and 200 mg lysozyme/kg such as 100 mg/kg lysozyme/kg per day.

In one embodiment, methods are provided as described herein, wherein the daily dosage is between 1 and 1000 μg lactoferricin/kg such as between 1 and 500 μg lactoferrin/kg, such as between 5 and 100 μg lactoferricin/kg such as 10 μg/kg lactoferricin per day.

In one embodiment an indicated daily dosage of a human cathelicidin is preferably from about 0.1 mg cathelicidin/kg body weight to about 10 mg cathelicidin/kg body weight, for example, administered in divided doses up to one, two or three times a day. In one embodiment an indicated daily dosage of a human lactoferrin is preferably from about 1 mg/kg body weight to about 1000 mg lactoferrin/kg body weight, for example, administered in divided doses up to one, two or three times a day.

In one embodiment an indicated daily dosage of a human lactoferricin is preferably from about 1 μg/kg body weight to about 1000 μg/kg body weight, for example, administered in divided doses up to one, two or three times a day.

In one embodiment an indicated daily dosage of a human lysozyme is preferably from about 1 mg lysozyme/kg body weight to about 1000 mg lysozyme/kg body weight, for example, administered in divided doses up to one, two or three times a day.

In certain embodiments, the pharmaceutical compositions of preferred embodiments can include a mammalian alfa defensin and/or a mammalian beta defensin and/or a mammalian cathelicidin and/or a mammalian lactoferrin/lactoferricin and/or a mammalian lysozyme, such as a human alfa defensin and/or a human beta defensin and/or a human cathelicidin and/or a human lactoferrin/lactoferricin and/or a human lysozyme, in an amount of about 0.01 mg or less to about 1500 mg or more per unit dosage form, preferably from about 0.01, 0.02, 0.03, 0.04, or 0.05 mg to about 150, 200, 250, 300, 350, 400, 450, 500, 600, 700, 800, 900, or 1000 mg, and more preferably from about 1, 2, 3, 4, 5, 6, 7, 8, 9, 10, 15, 20, or 25 mg to about 30, 35, 40, 45, 50, 55, 60, 65, 70, 75, 80, 85, 90, 95, or 100 mg. In certain embodiments, however, lower or higher dosages than those mentioned above may be preferred. Appropriate concentrations and dosages can be readily determined by one skilled in the art.

In certain embodiments, the pharmaceutical compositions of preferred embodiments include a mammalian alfa defensin, such as a human alfa defensin. In other embodiments, the pharmaceutical compositions of preferred embodiments include a mammalian beta defensin, such as a human beta defensin. In further embodiments, the pharmaceutical compositions of preferred embodiments include a mammalian alfa defensin and a mammalian beta defensin, such as a human alfa defensin and a human beta defensin, wherein the alfa and the beta defensins are present in equal amounts on a molarity basis or on a mg/mL basis. In other embodiments, the pharmaceutical compositions of preferred embodiments include a mammalian cathelicidin, such as a human cathelicidin. In other embodiments, the pharmaceutical compositions of preferred embodiments include a mammalian lactoferrin/lactoferricin, such as a human lactoferrin/lactoferricin. In other embodiments, the pharmaceutical compositions of preferred embodiments include a mammalian lysozyme, such as a human lysozyme.

In one embodiment, the mammalian alfa and/or beta defensin and/or cathelicidin and/or lactoferrin/lactoferricin and/or lysozyme is administered at least once daily, such as at least twice daily, for example at least 3 times daily.

Formulations for Oral or Parenteral Administration

Mammalian alfa defensins, beta defensins, cathelicidins, lactoferrins/lactoferricins and lysozymes can be employed therapeutically in compositions formulated for administration by any conventional route. In one embodiment, the administration is oral, buccal, sublingual, rectal, vaginal, intratracheal, intrapulmonary, intranasal, intracranial, subcutaneous, intravenous, dermal or transdermal. Preferably the administration is oral.

In one embodiment, the administration of at least one mammalian α-defensin and/or at least one mammalian β-defensin and/or at least one mammalian cathelicidin and/or at least one mammalian lactoferrin/lactoferricin and/or at least one mammalian lysozyme, according to the disclosed methods, is generally intranasal or intrapulmonary. Intranasal and intrapulmonary administration is normal for pulmonary drug delivery. In one embodiment, the administration of at least one mammalian α-defensin and/or at least one mammalian β-defensin and/or at least one mammalian cathelicidin and/or at least one mammalian lactoferrin/lactoferricin and/or at least one mammalian lysozyme, according to the disclosed methods, is oral.

In one embodiment, the administration of at least one mammalian α-defensin and/or at least one mammalian β-defensin and/or at least one mammalian cathelicidin and/or at least one mammalian lactoferrin/lactoferricin and/or at least one mammalian lysozyme, according to the disclosed methods, is subcutaneous or intravenous.

Within some embodiments, compositions of preferred embodiments may be formulated as a lyophilizate, utilizing appropriate excipients that provide stability as a lyophilizate, and subsequently after rehydration. Pharmaceutical compositions containing a mammalian alfa defensin and/or a mammalian beta defensin and/or a mammalian cathelicidin and/or a mammalian lactoferrin/lactoferricin and/or a mammalian lysozyme, such as a human alfa defensin and/or a human beta defensin and/or human cathelicidin and/or human lactoferrin/lactoferricin and/or human lysozyme, can be manufactured according to conventional methods, e.g., by mixing, granulating, coating, dissolving or lyophilizing processes. In a preferred embodiment, pharmaceutical compositions containing a mammalian alfa defensin and/or a mammalian beta defensin and/or a mammalian cathelicidin and/or a mammalian lactoferrin/lactoferricin and/or a mammalian lysozyme are formulated as a sterile and isotonic solution.

Pharmaceutically acceptable carriers and/or diluents are familiar to those skilled in the art. For compositions formulated as liquid solutions, acceptable carriers and/or diluents include saline and sterile water should be included, and the composition may optionally include antioxidants, buffers, bacteriostats, and other common additives. The disclosed compositions may be formulated in a wide variety of formulations for oral administration. Solid form preparations may include powders, tablets, drops, capsules, cachets, lozenges, and dispersible granules. Other forms suitable for oral administration may include liquid form preparations including emulsions, syrups, elixirs, aqueous solutions, aqueous suspensions, toothpaste, gel dentifrice, chewing gum, or solid form preparations which are intended to be converted shortly before use to liquid form preparations, such as solutions, suspensions, and emulsions.

The disclosed compositions may be formulated in a wide variety of formulations for buccal, sublingual, oral, rectal, vaginal, dermal, transdermal, intracranial, subcutaneous or intravenous administration. The formulation can contain (in addition to a mammalian alfa defensin and/or a mammalian beta defensin and/or a mammalian cathelicidin and/or a mammalian lactoferrin/lactoferricin and/or a mammalian lysozyme, and other optional active ingredients) carriers, fillers, disintegrators, flow conditioners, sugars and sweeteners, fragrances, preservatives, stabilizers, wetting agents, emulsifiers, solubilizers, salts for regulating osmotic pressure, buffers, diluents, dispersing and surface-active agents, binders, lubricants, and/or other pharmaceutical excipients as are known in the art. One skilled in this art may further formulate mammalian alfa defensins, mammalian beta defensins, mammalian cathelicidins, mammalian lactoferrins/lactoferricins and mammalian lysozymes in an appropriate manner, and in accordance with accepted practices, such as those described in Remington's Pharmaceutical Sciences, Gennaro (1990).

A mammalian alfa defensin, a mammalian beta defensin, a mammalian cathelicidin, a mammalian lactoferrin/lactoferricin and a mammalian lysozyme, such as a human alfa defensin, a human beta defensin, a human cathelicidin, a human lactoferrin/lactoferricin and a human lysozyme, can be used alone, or in combination therapies with one, two, or more other pharmaceutical compounds or drug substances, for example with surfactant, prebiotics, probiotics, glucocorticoids, antibiotics, immunosuppressants, GLP-2 or GLP-2 analogs or a combination of these and/or with one or more pharmaceutically acceptable excipient(s).

Airway Administration

Airway administration may be used for administering the compositions of the invention. The term “intrapulmonary administration” as used herein refers to a topical administration to the lungs. When used herein the terms “intratracheal, intrabronchial or intra alveolar administration” include all forms of such administration whereby defensins, cathelicidins, lactoferrins, lactoferricins and/or lysozymes is applied into the trachea, the bronchi or the alveoli, respectively, whether by instillation of a solution of defensins, cathelicidins, lactoferrins, lactoferricins and/or lysozymes, by applying defensins, cathelicidins, lactoferrins, lactoferricins and/or lysozymes in a powder form, or by allowing defensins, cathelicidins, lactoferrins, lactoferricins and/or lysozymes to reach the relevant part of the airway by inhalation of defensins, cathelicidins, lactoferrins, lactoferricins and/or lysozymes as an aerosolized or nebulized solution or suspension or inhaled powder or gel, with or without added stabilizers or other excipients.

Methods of intrabronchial/alveolar administration include, but are not limited to, bronchoalveolar lavage (BAL) according to methods well known to those skilled in the art, using as a lavage fluid a physiologically acceptable composition in which defensins, cathelicidins, lactoferrins, lactoferricins and/or lysozymes has been dissolved or indeed by any other effective form of intrabronchial administration including the use of inhaled powders containing defensins, cathelicidins, lactoferrins, lactoferricins and/or lysozymes in dry form, with or without excipients, or the direct application of defensins, cathelicidins, lactoferrins, lactoferricins and/or lysozymes, in solution or suspension or powder form during bronchoscopy. Methods for intratracheal administration include, but are not limited to, blind tracheal washing with a similar solution of dissolved defensins, cathelicidins, lactoferrins, lactoferricins and/or lysozymes or defensins, cathelicidins, lactoferrins, lactoferricins and/or lysozymes suspension, or the inhalation of nebulized fluid droplets containing dissolved defensins, cathelicidins, lactoferrins, lactoferricins and/or lysozymes or defensins, cathelicidins, lactoferrins, lactoferricins and/or lysozymes suspension obtained by use of any nebulizing apparatus adequate for this purpose.

In another embodiment, intratracheal, intrabronchial or intra alveolar administration does not include inhalation of the product but the instillation or application of a solution of defensins, cathelicidins, lactoferrins, lactoferricins and/or lysozymes or a powder or a gel containing defensins, cathelicidins, lactoferrins, lactoferricins and/or lysozymes into the trachea or lower airways.

Other preferred methods of administration may include using the following devices:
1. Pressurized nebulizers using compressed air/oxygen mixture
2. Ultrasonic nebulizers
3. Electronic micropump nebulizers
4. Metered dose inhaler (MDI)
5. Dry powder inhaler systems (DPI),

The aerosol may be delivered via a) facemasks or b) via endotracheal tubes in intubated patients during mechanical ventilation (device 1, 2 and 3). The devices 4 and 5 can also be used by the patient without assistance provided that the patient is able to self-activate the aerosol device.

Preferred concentrations for a solution comprising defensins, cathelicidins, lactoferrins, lactoferricins and/or lysozymes and/or functional homologues or variants of defensins, cathelicidins, lactoferrins, lactoferricins and/or lysozymes are in the range of from about 0.1 μg to 1000 μg per ml solution, such as in the range of from about 0.1 μg to 250 μg per ml solution.

Pharmaceutical Composition for Intrapulmonary Administration

Pharmaceutical compositions or formulations for use in the present disclosure include defensins, cathelicidins, lactoferrins, lactoferricins and/or lysozymes in combination with, preferably dissolved in, a pharmaceutically acceptable carrier, preferably an aqueous carrier or diluent, or carried to the lower airways as a pegylated preparation or as a liposomal or nanoparticle preparation administered as an aerosol via inhalation, or as a lavage fluid administered via a bronchoscope as a bronchoalveloar lavage or as a blind intratracheal wash or lavage. A variety of aqueous carriers may be used, including, but not limited to 0.9% saline, buffered saline, physiologically compatible buffers and the like. The compositions may be sterilized by conventional techniques well known to those skilled in the art. The resulting aqueous solutions may be packaged for use or filtered under aseptic conditions and freeze-dried, the freeze-dried preparation being dissolved in a sterile aqueous solution prior to administration

In one embodiment a freeze-dried defensin, cathelicidin, lactoferrin, lactoferricin and/or lysozyme preparation may be pre-packaged for example in single dose units. In an even more preferred embodiment the single dose unit is adjusted to the patient.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

The formulations may contain pharmaceutically acceptable carriers and excipients including microspheres, liposomes, microcapsules, nanoparticles or the like. Conventional liposomes are typically composed of phospholipids (neutral or negatively charged) and/or cholesterol. The liposomes are vesicular structures based on lipid bilayers surrounding aqueous compartments. They can vary in their physiochemical properties such as size, lipid composition, surface charge and number and fluidity of the phospholipids bilayers. The most frequently used lipid for liposome formation are: 1,2-Dilauroyl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC), 1,2-Distearoyl-sn-Glycero-3-Phosphocholine (DSPC), 1,2-Dioleoyl-sn-Glycero-3-Phosphocholine (DOPC), 1,2-Dimyristoyl-sn-Glycero-3-Phosphoethanolamine (DMPE), 1,2-Dipalmitoyl-sn-Glycero-3-Phosphoethanolamine (DPPE), 1,2-Dioleoyl-sn-Glycero-3-Phosphoethanolamine (DOPE), 1,2-Dimyristoyl-sn-Glycero-3-Phosphate (Monosodium Salt) (DMPA), 1,2-Dipalmitoyl-sn-Glycero-3-Phosphate (Monosodium Salt) (DPPA), 1,2-Dioleoyl-sn-Glycero-3-Phosphate (Monosodium Salt) (DOPA), 1,2-Dimyristoyl-sn-Glycero-3-[Phospho-rac-(1-glycerol)] (Sodium Salt) (DMPG), 1,2-Dipalmitoyl-sn-Glycero-3-[Phospho-rac-(1-glycerol)] (Sodium Salt) (DPPG), 1,2-Dioleoyl-sn-Glycero-3-[Phospho-rac-(1-glycerol)] (Sodium Salt) (DOPG), 1,2-Dimyristoyl-sn-Glycero-3-[Phospho-L-Serine] (Sodium Salt) (DMPS), 1,2-Dipalmitoyl-sn-Glycero-3-[Phospho-L-Serine) (Sodium Salt) (DPPS), 1,2-Dioleoyl-sn-Glycero-3-[Phospho-L-Serine] (Sodium Salt) (DOPS), 1,2-Dioleoyl-sn-Glycero-3-Phosphoethanolamine-N-(glutaryl) (Sodium Salt) and 1,1′,2,2′-Tetramyristoyl Cardiolipin (Ammonium Salt). Formulations composed of DPPC in combination with other lipids or modifiers of liposomes are preferred e.g. in combination with cholesterol and/or phosphatidylcholine.

Long-circulating liposomes are characterized by their ability to extravasate at body sites where the permeability of the vascular wall is increased. The most popular way of producing long-circulating liposomes is to attach hydrophilic polymer polyethylene glycol (PEG) covalently to the outer surface of the liposome. Some of the preferred lipids are: 1,2-Dipalmitoyl-sn-Glycero-3-Phosphoethanolamine-N-[Methoxy(Polyethylene glycol)-2000] (Ammonium Salt), 1,2-Dipalmitoyl-sn-Glycero-3-Phosphoethanolamine-N-[Methoxy(Polyethylene glycol)-5000] (Ammonium Salt), 1,2-Dioleoyl-3-Trimethylammonium-Propane (Chloride Salt) (DOTAP).

Possible lipids applicable for liposomes are supplied by Avanti, Polar Lipids, Inc, Alabaster, Ala. Additionally, the liposome suspension may include lipid-protective agents which protect lipids against free-radical and lipid-peroxidative damage on storage. Lipophilic free-radical quenchers, such as alpha-tocopherol and water-soluble iron-specific chelators, such as ferrioxianine, are preferred.

A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028, all of which are incorporated herein by reference. Another method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous solution of the targeted drug and the targeting component and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Micelles are formed by surfactants (molecules that contain a hydrophobic portion and one or more ionic or otherwise strongly hydrophilic groups) in aqueous solution.

Common surfactants well known to one of skill in the art can be used in the micelles of the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are nonionic polyoxyethylene and polyoxypropylene detergents compatible with IV injection such as, TWEEN-80, PLURONIC F-68, n-octyl-beta-D-glucopyranoside, and the like. In addition, phospholipids, such as those described for use in the production of liposomes, may also be used for micelle formation.

It is an aspect of the present disclosure to provide method for preventing or treating a mucosal disorder in a preterm infant, wherein the mucosal disorder is selected from the group consisting of: necrotizing enterocolitis, acute and prolonged diarrhea, short bowel syndrome, respiratory illness, lung inflammation, respiratory tract infection, respiratory failure, pneumonia, obstructive apnea, bronchopulmonary dysplasia, respiratory distress syndrome, and primary atelectasis, the method comprising administering a composition comprising at least one antimicrobial peptide selected from the group consisting of: α-defensins and 3-defensins to a preterm infant or to a woman about to give birth to a preterm infant.

A further aspect of the present disclosure is to provide the method as presented herein, wherein the mucosal disorder is in the gut and/or in the lung.

In one embodiment, the mucosal disorder is in the gut and is selected from the group consisting of: necrotizing enterocolitis, acute and prolonged diarrhea, and short bowel syndrome.

In one embodiment, the mucosal disorder is in the lung and is selected from the group consisting of: respiratory illness, lung inflammation, respiratory tract infection, respiratory failure, pneumonia, obstructive apnea, bronchopulmonary dysplasia, respiratory distress syndrome, and primary atelectasis.

In one aspect, the present disclosure provides a method for maturing the mucosal defense and rebalancing the immune system in the gut and lung in a preterm infant, the method comprising administering a composition comprising at least one antimicrobial peptide selected from the group consisting of α-defensins, 3-defensins, cathelicidins, lactoferrins, lactoferricins and lysozymes to a preterm infant or to a woman about to give birth to a preterm infant (preterm labor).

In one aspect, the present disclosure provides a method for preventing or treating sepsis, respiratory illness, necrotizing enterocolitis, acute and prolonged diarrhea, short bowel syndrome, impaired neurodevelopment and extra uterine growth restriction in a preterm infant, the method comprising administering a composition comprising at least one antimicrobial peptide selected from the group consisting of α-defensins, β-defensins, cathelicidins, lactoferrins, lactoferricins and lysozymes to a preterm infant or to a woman about to give birth to a preterm infant.

In one aspect, the present disclosure provides a method for improving intestinal health i.e. decreasing myeloperoxidase activity in a preterm infant, the method comprising administering a composition comprising at least one antimicrobial peptide selected from the group consisting of α-defensins, β-defensins, cathelicidins, lactoferrins, lactoferricins and lysozymes to a preterm infant or to a woman about to give birth to a preterm infant.

In one aspect, the present disclosure provides a method for preventing or treating necrotizing enterocolitis, gut immaturity, dysmolality, increased gut permeability, abnormal microbiota, and sepsis in a preterm infant, the method comprising improving intestinal health by administering a composition comprising at least one antimicrobial peptide selected from the group consisting of α-defensins, 3-defensins, cathelicidins, lactoferrins, lactoferricins and lysozymes to a preterm infant or to a woman about to give birth to a preterm infant.

In one aspect, the present disclosure provides a method for prevention or treatment of short bowel syndrome in a preterm infant, said method comprising administering a composition comprising at least one antimicrobial peptide selected from the group consisting of α-defensins, β-defensins, cathelicidins, lactoferrins, lactoferricins and lysozymes and potentially one or more GLP-2 or GLP-2 analogs to a preterm infant or to a woman about to give birth to a preterm infant.

In one aspect, the present disclosure provides a method for prevention or treatment of extrauterine growth restriction in a preterm infant, said method comprising improving intestinal health by administering a composition comprising at least one antimicrobial peptide selected from the group consisting of α-defensins, 3-defensins, cathelicidins, lactoferrins, lactoferricins and lysozymes to a preterm infant or to a woman about to give birth to a preterm infant.

In one aspect, the present disclosure provides a method for prevention or reduction of neurodevelopmental impairment, white-matter injury, cerebral palsy, mental retardation and sensory impairments in a preterm infant, said method comprising administering a composition comprising at least one antimicrobial peptide selected from the group consisting of α-defensins, β-defensins, cathelicidins, lactoferrins, lactoferricins and lysozymes to a preterm infant or to a woman about to give birth to a preterm infant.

In one aspect, the present disclosure provides a method for prevention of respiratory illness, lung inflammation, respiratory tract infection, respiratory failure, pneumonia and sepsis in a preterm infant, said method comprising administering a composition comprising at least one antimicrobial peptide selected from the group consisting of α-defensins, β-defensins, cathelicidins, lactoferrins, lactoferricins and lysozymes to a preterm infant or to a woman about to give birth to a preterm infant.

In one aspect, the present disclosure provides a method of reducing histological lung inflammation and inflammatory cell count in bronchoalveolar lavage fluid in a preterm infant, said method comprising administering a composition comprising at least one antimicrobial peptide selected from the group consisting of α-defensins, β-defensins, cathelicidins, lactoferrins, lactoferricins and lysozymesto a preterm infant or to a woman about to give birth to a preterm infant.

In one aspect, the present disclosure provides a method for rebalancing the immune response normalizing the inflammatory response and preventing cytokine storm through normalization of the inflammatory cytokine production in a preterm infant, said method comprising administering a composition comprising at least one antimicrobial peptide selected from the group consisting of α-defensins, β-defensins, cathelicidins, lactoferrins, lactoferricins and lysozymesto a preterm infant or to a woman about to give birth to a preterm infant.

In one aspect, the present disclosure provides a method of increasing pulmonary compliance, reducing airway hyper responsiveness, and/or increasing the peak expiratory flow in a preterm infant, said method comprising administering a composition comprising at least one antimicrobial peptide selected from the group consisting of α-defensins, β-defensins, cathelicidins, lactoferrins, lactoferricins and lysozymesto a preterm infant or to a woman about to give birth to a preterm infant.

In one aspect, the present disclosure provides a method of increasing lung function and pulmonary compliance, reducing airway hyper responsiveness, and/or increasing the peak expiratory flow in a preterm infant, said method comprising administering a composition comprising at least one antimicrobial peptide selected from the group consisting of α-defensins, β-defensins, cathelicidins, lactoferrins, lactoferricins and lysozymesto a preterm infant or to a woman about to give birth to a preterm infant.

In one aspect, the present disclosure provides a method for prevention and/or treatment of infection following preterm ruptured membranes, maternal infection, Caesarian delivery, perinatal and postnatal broad spectrum antibiotic exposure as well as exposure to other gut-modifying medications in a preterm infant, said method comprising administering a composition comprising at least one antimicrobial peptide selected from the group consisting of α-defensins, β-defensins, cathelicidins, lactoferrins, lactoferricins and lysozymes to a preterm infant or to a woman about to give birth to a preterm infant.

In one aspect, the present disclosure provides a method for increasing gene richness, increasing the number of phylae, increasing the bacterial presence, increasing the bacterial abundance, increasing the butyrate production and/or decreasing the acetate production from gut or lung microbiota in a preterm infant, said method comprising administering a composition comprising at least one antimicrobial peptide selected from the group consisting of α-defensins, β-defensins, cathelicidins, lactoferrins, lactoferricins and lysozymes to a preterm infant or to a woman about to give birth to a preterm infant.

In one aspect, the present disclosure provides a method for maturing, maintaining and/or stabilizing a normal microbiota in the gut or lung or skin in a preterm infant, said method comprising administering a composition comprising at least one antimicrobial peptide selected from the group consisting of α-defensins, β-defensins, cathelicidins, lactoferrins, lactoferricins and lysozymes to a preterm infant or to a woman about to give birth to a preterm infant.

In one aspect, the present disclosure provides a method of increasing the abundance of Allobaculum, Alloprevotella, Akkermansia, Barnesiella, Bifidobacteriaceae, Faecalibacterium, Lachnospira, Rothia and Veillonella in the gut of a preterm infant, said method comprising administering a composition comprising at least one antimicrobial peptide selected from the group consisting of α-defensins, 3-defensins, cathelicidins, lactoferrins, lactoferricins and lysozymesto a preterm infant or to a woman about to give birth to a preterm infant.

In one aspect, the present disclosure provides a method for increasing food uptake and weight gain in a preterm infant, said method comprising administering a composition comprising at least one antimicrobial peptide selected from the group consisting of α-defensins, β-defensins, cathelicidins, lactoferrins, lactoferricins and lysozymes to a preterm infant or to a woman about to give birth to a preterm infant.

### EXAMPLES

### Example 1. Modulation of Gut Microbiota by Prophylactic Treatment with Defensins

Materials and Methods:

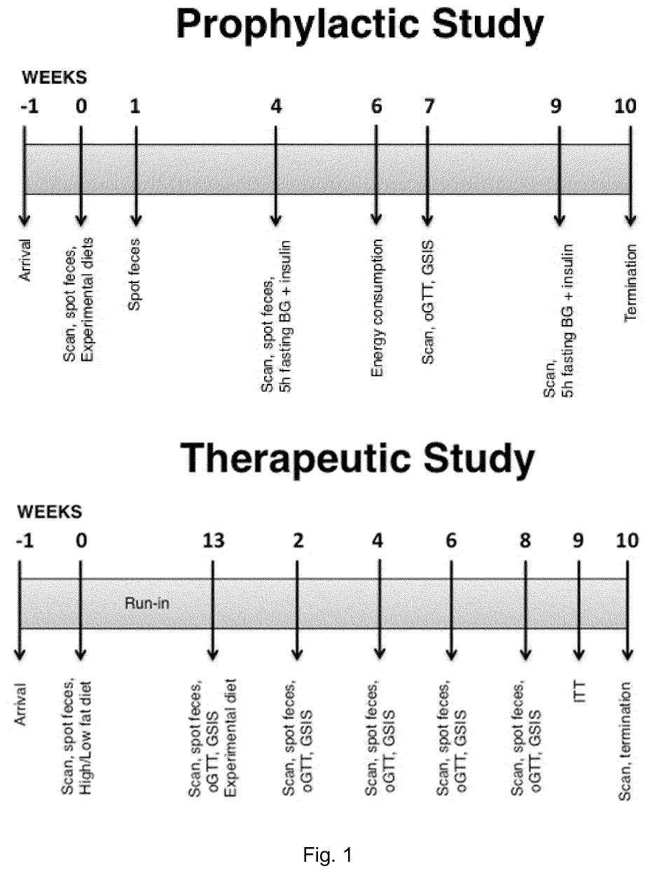

The overall experimental design is shown in FIG. 1, top panel “Prophylactic study”.

Mice:

Mice were housed in trios, 4 cages per group. Feed intake was registered daily just before lights were turned off at 6 pm. Individual mice were subjected to experimental procedures in altered order both group and cage wise. Mice were kept at room temperature under a 12-hour light/dark cycle at SPF standard conditions.

Diets:

For dosing, the average weight was estimated to be 25 grams per mouse. Mice eat approximately 3 grams of feed per mouse per day.

Treatment Regime:

Mice were fed either a high fat diet (HFD) or a low fat (LF) control diet. The HFD contained 4 subgroups; a) hBD-2, b) HD5, c) hBD-2/HD5 and d) standard HFD without supplementation of defensins. Defensin concentration was 1.2 mg hBD-2 per kg mouse per day. HD5 was given in equimolar concentration to hBD-2. The combinatory group was given 50% hBD-2+50% HD5, hence a total amount of defensins equivalent to the remaining test groups.

Tests:

Microbial analyses were carried out to study the microbiota of the intestine. Longitudinal 16S characterization was conducted on 4 paired samples from 60 mice, 240 samples in total. Each mouse was sampled prior to diet change, 1 week post diet change, 4 weeks post diet change and at termination, thus ensuring a thorough characterization of the faecal microbiota as a result of defensin treatment.

Results:

Weight Change.

While the food intake was similar in all three experimental diet groups, both High Fat Diet (HFD) groups (a) treated with hBD-2 and d) standard HFD without treatment) gained significantly more weight than the Low Fat Diet (LFD) reference group over the 10 week study period (*p<0.0001, 2-way ANOVA, Tukey Post Test). The HFD plus hBD-2 group, however, gained significantly less weight than the HFD reference group (*p=0.0028).

Microbiota.

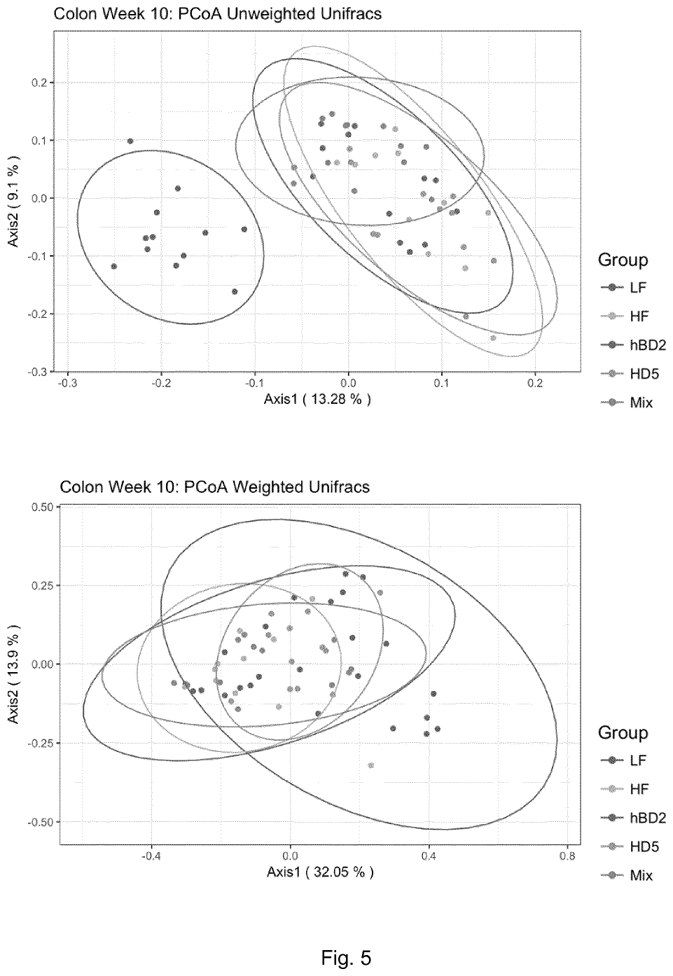

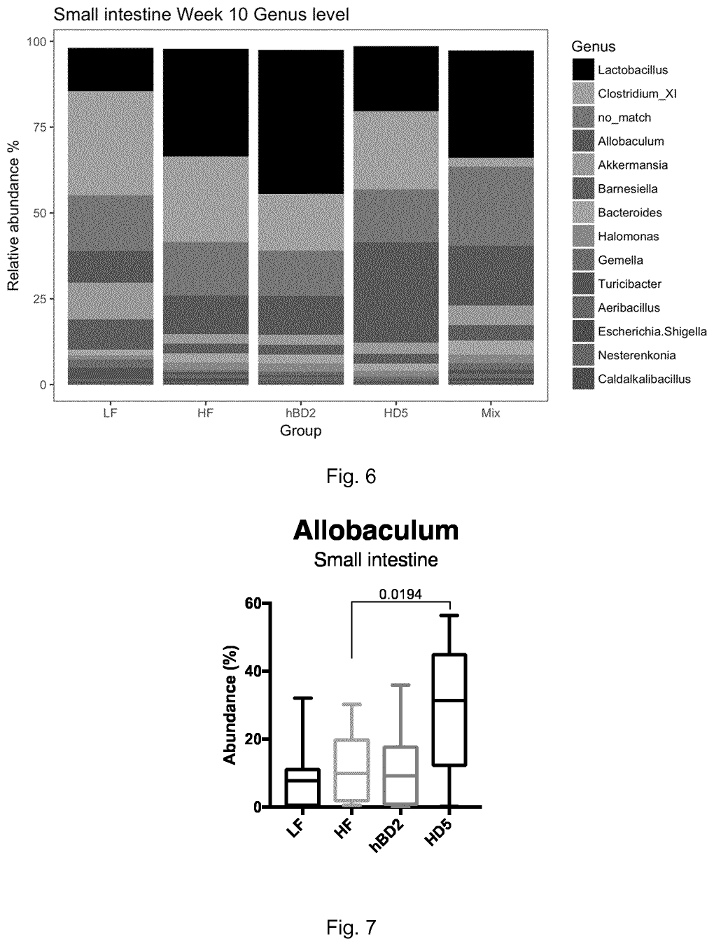

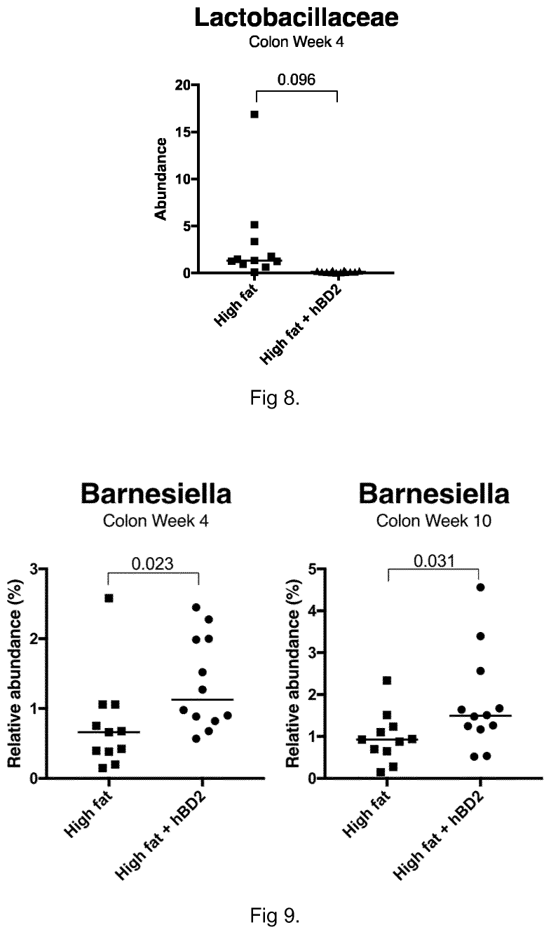

hBD-2 affected primarily the microbial presence, whereas HD5 and hBD-2+HD5 affected primarily the microbial abundance (FIGS. 5 and 6). Presence describes the number of different bacteria present. Increased presence signifies a more diverse microbiota and has in many publications over the past decade been associated with a healthier microbiota and in some cases with specific disease improvement in animal models. Abundance describes the number of bacteria of a given strain present. Ideally, one would like to increase the abundance of “good” bacteria such as Barnesiella, prevotella species and Bifidobacteriaceae, which have been identified as important species, while decreasing the abundance of e.g. pathogens. A statistically significant increase of abundance of Allobaculum was seen in the small intestine following prophylaxis with HD5 (p<0.02; FIG. 7). Allobaculum is a short chain fatty acid producing species. A trend towards lower abundance of Lactobacillaceae was observed in colon following prophylactic treatment with hBD-2 (p=0.1; FIG. 8). A statistically significant increase in abundance of Barnesiella in the colon was observed following prophylactic treatment with hBD-2 (p<0.03; FIG. 9). Barnesiella is a bacteria that has been found to be able to eliminate and protect against the intestinal dominance of antibiotic-resistant pathogenic bacteria that can be observed in hospitalized patients. The abundance of Barnesiella corresponds with the amount of several immunoregulatory cells. The higher the level of Barnesiella in the colon, the more marginal zone B cells and invariant natural killer T cells enumerated in the spleen and liver. Furthermore, a direct association between a change in microbial composition in favor of Barnesiella and the resistance to arthritis has been demonstrated in mice. In the development of colitis in IL-10−/− mice, higher levels of a Barnesiella phylotype correlated with lower activity levels of the disease.

Conclusions:

Premature infants are almost exclusively born via Caesarian sectio. The procedure is performed under sterile conditions and the infants are thus born without a natural microbial population especially of the large mucosal surfaces in the intestines, lungs and skin. The only approved treatment of premature infants today as per the recent Cochrane analysis of therapies (Alfaleh and Annebrees, 2014) are probiotics based on the theory that the mucosal surfaces of these infants will have to be populated by microbes via external sources to establish a microbiota. A foetus starts to produce defensins from the beginning of the third trimester to prepare the mucosal surfaces for the massive bacterial colonization following birth via the natural birth canal and the ELBW premature infants are thus born without any or only a very small natural production of defensins. The above example serves to demonstrate that defensins in the intestines have a profound influence in promoting important commensal bacteria and maintaining a normal microbiota and that the microbiota modulating effect of two different defensins is different.

### Example 2. Modulation of Gut Microbiota by Interventional Treatment with Defensins

Materials and Methods:

The overall design of the study is shown in FIG. 1, lower panel, “Therapeutic study”.

Mice and Diets.

The experiment elucidates the effect of hBD-2 and HD5 on the microbiota in diet-induced obese mice. A run-in period of 13 weeks where mice were fed a very HFD (60% energy from fat) preceded the intervention. Only mice meeting the criteria of a minimum of 12 gram weight gain (approximately 50% of initial bodyweight) during the run-in period were included in the final analyses. Mice that did not meet these criteria stayed in their respective cages as hierarchy ‘keepers’. They were exposed to all experimental tests, but excluded from the analyses.

Treatment Regimen.

Before the intervention all mice were MR scanned. Cages of mice were allocated to experimental groups based on their fat mass. All subsequent measures were paired with data from the same mouse before the intervention. A LFD (low fat diet) reference group was running in parallel. As controls for the intervention 2 additional groups were included: a) very HFD and b) LFD. Experimental mice stayed on the very HFD during the intervention. The mice were on the experimental diet for 10 weeks. They were co-housed throughout the experiment, 4 mice per cage, 3 cages per group. All tests ran over 3 days, 1 cage per group per day.

Tests:

Microbial analyses were carried out to study the microbiota of the intestine. Longitudinal 16S characterization was conducted on 4 paired samples from 60 mice, 240 samples in total. Each mouse was sampled prior to diet change, 1 week post diet change, 4 weeks post diet change and at termination, thus ensuring a thorough characterization of the faecal microbiota as a result of defensin treatment.

Results:

Weight Change—hBD-2.

The standard high fat diet (HFD) fed groups had an equal food intake throughout the entire study period and had the same weight development with equal fat and lean mass the first 13 weeks, thus having the same starting point prior to the dietary intervention. The weight gain was significantly larger than in the low fat diet fed (LFD) group (*p<0.05 2-way ANOVA). After the dietary intervention the HFD groups continued to increase in weight, however the HFD plus hBD-2 group tended to gain less weight the first 4 weeks post dietary intervention, although not significant (*p=0.07, 2-way ANOVA). From week 4 to the end of the study period the HFD plus hBD-2 group gained similar weight as the standard HFD group (*p=0.82, 2-way ANOVA).

Weight Change—HD5.

All HFD fed groups had the same food intake during the study period and equal weight gain during the run-in period of 13 weeks. After dietary intervention the group fed HFD plus HD5 gained significantly less weight than the HFD control (*p<0.05, 2-way ANOVA). In addition, a tendency of decreasing fat percentage in the HFD plus HD5 group was observed, and a significantly lower fat percentage in the HFD plus HD5 was measured 4 weeks after dietary change in comparison to the HFD control (*p=0.009 2-way ANOVA).

Microbiota.

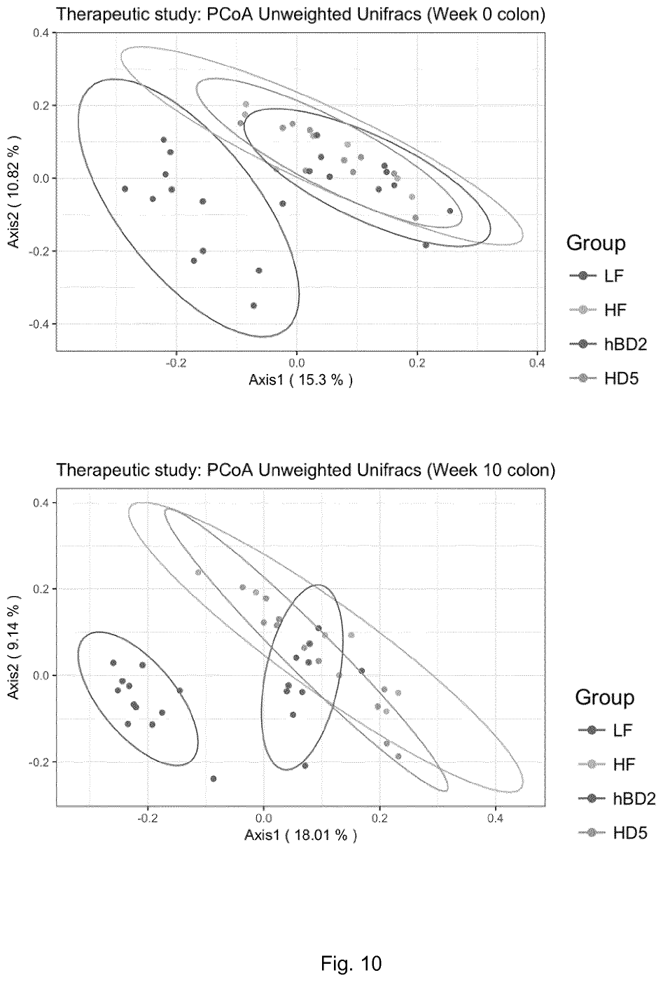

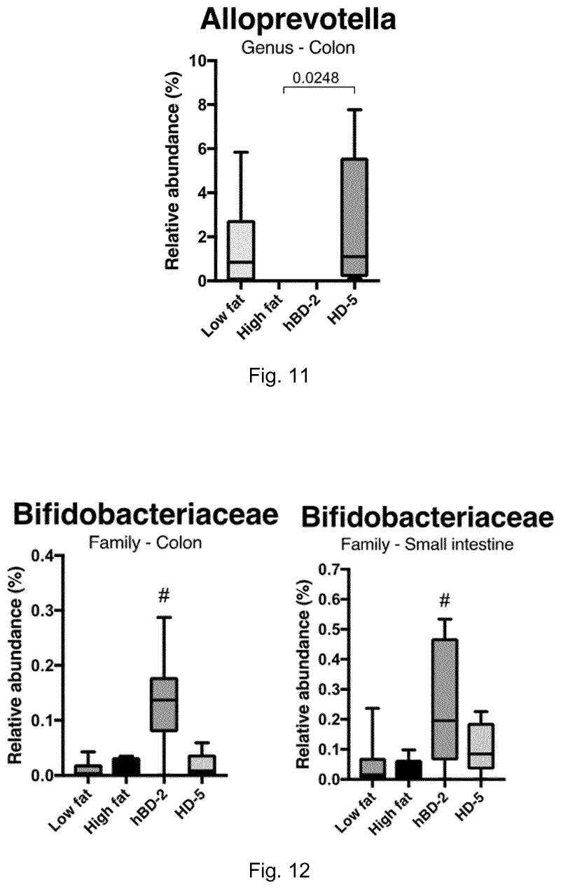

Both defensins were shown to have a profound influence on the bacterial presence as well as bacterial absence i.e. the capability to change the microbiota fascilitating the colonization of “good” bacteria and decreasing the colonization of “bad” bacteria or pathogens (FIG. 10). HD5 increased the abundance of Alloprevotella statistically significantly in the colon (p<0.02) whereas hBD-2 had no influence on Alloprevotella abundance (FIG. 11). hBD-2 dramatically and statistically significant increased the relative abundance of Bifidobacteriaceae both in the small intestine and in the colon (p<0.0001 and p<0.04 respectively; FIG. 12). There was a trend towards HD5 increasing the abundance of Bifidobacteriaceae in the small intestine (FIG. 12).

Conclusions:

Premature infants are almost exclusively born via Caesarian sectio. The procedure is performed under sterile conditions and the infants are thus born without a natural microbial population especially of the large mucosal surfaces in the intestines, lungs and skin. The only approved treatment of premature infants today as per the recent Cochrane analysis of therapies (Alfaleh and Annebrees, 2014) are probiotics based on the theory that the mucosal surfaces of these infants will have to be populated by microbes via external sources to establish a natural microbiota. A foetus starts to produce defensins from the beginning of the third trimester to prepare the mucosal surfaces for the massive bacterial colonization following birth via the natural birth canal. The above example thus serves to demonstrate that defensins in the intestines have a profound influence in establishing and maintaining a normal microbiota and that the microbiota modulating effect of two different defensins is different.

### Example 3. Method to Determine the Efficacy of Prophylactic Treatment with Oral Mammalian α- and β-Defensins in a Premature Piglet Model of Necrotizing Enterocolitis

Materials and Methods:

Treatment Regime:

24 preterm piglets are delivered by caesarean section on day 105 of gestation. The newborn piglets are immediately transferred to incubators with regulated temperature and oxygen supply. When respiration has stabilized, the piglets are fitted with umbilical and orogastric catheters. All piglets are initially provided parenteral nutrition via an esophageal tube. The enteral formula diet is made from three commercially available products used for feeding infants 0-2 years of age. Piglets are stratified according to birth weight and sex and allocated into a control and an intervention group receiving hBD-2.

Tests:

Signs of discomfort or weakness (unwillingness to stand, cold extremities, distended abdomen, dehydration, pale skin color, diarrhea and bloody diarrhea) are recorded. The piglets are weighed every day and their food intake recorded.

An in vivo intestinal permeability test with lactulose and mannitol is performed.

On day 5, all piglets are euthanized, tissue is collected, and a macroscopic NEC scoring system applied. Briefly each of the 5 regions (stomach, proximal, mid and distal small intestine and the colon) of the gastrointestinal tract are macroscopically evaluated for pathological changes, indicative of inflammation and necrosis. The lesions are graded as follows: 1=absence of lesions; 2=local hyperemia; 3=hyperemia, extensive edema and local haemorrhage; 4=extensive haemorrhage; 5=local necrosis and pneumatosis intestinalis and 6=extensive transmural necrosis and pneumatosis intestinalis. An animal is designated as NEC positive when a minimum disease score of 3 in at least one region is observed.

For determination of mucosal proportion, a 10 cm segment from each small intestinal region is removed and slit along its length. The mucosa is gently scraped off with a plastic slide, and the proportion of mucosa determined on a wet weight basis. The weight of the heart, lungs, liver, kidneys, spleen and stomach are determined on a wet weight basis.

Samples of PFA-fixed intestine from proximal and distal regions of the small intestine and colon are embedded in paraffin and stained with HE for histomorphology and histopathology. For quantification of goblet cells in the distal small intestine and colon, tissue slides are stained with Alcian Blue and Periodic Acid Schiff.

As a measure of inflammation, tissue myeloperoxidase activity is assayed. IL-1β, IL-6, IL-8 and TNF-α are determined on tissue samples from the distal small intestine. Brush border enzyme activity is measured in snap-frozen samples from the proximal, middle and distal regions of the small intestine from each piglet.

### Example 4. Efficacy of s.c. hBD-2 Versus s.c. Anti TNF-α (Enbrel) and Intraperitoneal Dexamethasone in a Therapeutic, Murine, 14 Weeks, CD4+CD25+ T Cell Transfer Induced Colitis Model

Materials and Methods:

Treatment regime: 70 female BALB/c mice housed in groups of five per cage were allocated to 7 different treatment groups. Colitis was induced in SCID mice by transplantation of CD4+CD25 Tcells from BALB/c mice. Lymphocytes isolated from spleen and lymph nodes from BALB/c mice were subjected to negative selection of CD4+ Tcells. Afterwards, CD4+CD25+ cells were positively isolated by binding to the beads from the CD4+ Tcell suspensions and the CD4+CD25− were collected from the supernatant. The animals were treated with hBD-2, 0.1 mg/kg s.c. once daily 86 consecutive days from day 7 (OD); 1 mg/kg s.c. OD or 3 mg/kg s.c. OD; with mouse anti TNFα 100 μg/mouse s.c. twice weekly (enbrel); with dexamethasone 0.3 mg/kg intraperitoneally OD (dex); with PBS s.c. as vehicle from day 7 and 86 consecutive days (vehicle) or sham treated (sham).

Treatment Groups:
1.0.1 mg/kg hBD-2, once daily subcutaneously (n=11)
2. 1 mg/kg hBD-2, once daily subcutaneously (n=11)
3. 3 mg/kg hBD-2, once daily subcutaneously (n=11)
4. PBS (hBD-2 vehicle), once daily subcutaneously (n=11)
5. 0.3 mg/kg Dexamethasone, once daily intraperitoneally (n=11)
6. 100 μg/mouse soluble human 75-kilodalton TNF receptor linked to Fc portion of IgG1 (Enbrel), twice a week subcutaneously (n=11)
7. Sham treated group (n=6)

Tests:

Clinical assessment was based on body weight loss, stool consistency and presence of blood per rectum. The animals were sacrificed on day 95 and colon removed for assessment of weight and myeloperoxidase activity.

Results:

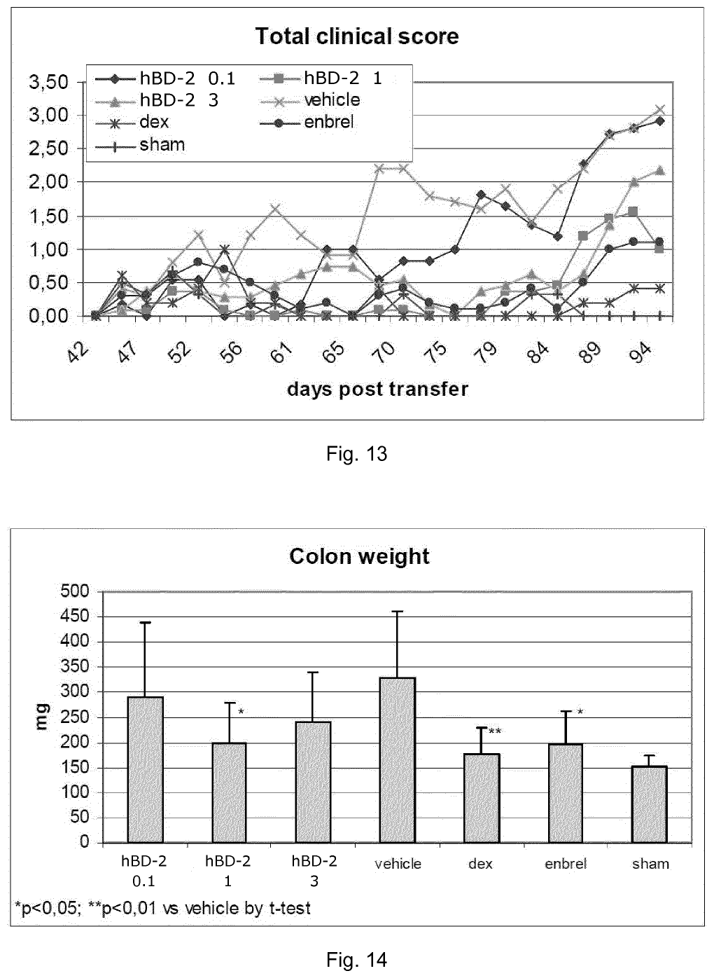

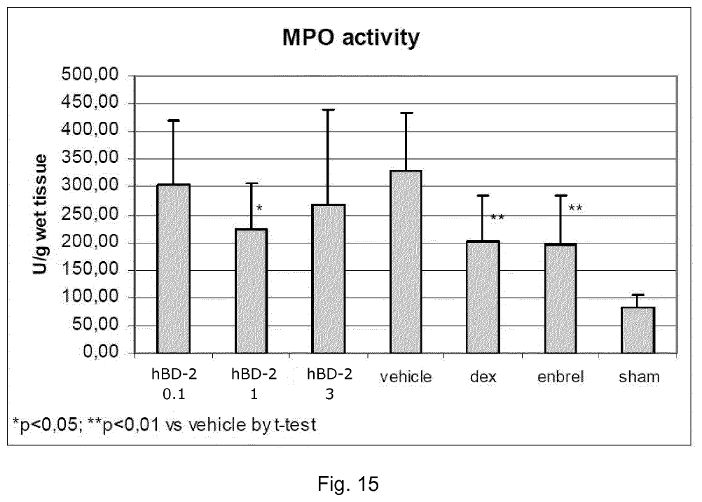

A statistically significant and similar effect on clinical score was observed for hBD-2 1 mg/kg s.c. OD, for Dexamethasone 0.3 mg/kg i.p. OD and for Enbrel (FIG. 13). Similarly these three dosing regimens showed a statistically significant and similar effect on colon weight (FIG. 14) and myeloperoxidase activity (FIG. 15).

Conclusions:

DSS induces a type of colitis in mice that shares a number of characteristics with necrotizing enterocolitis seen in human preterm infants. This example serves to demonstrate that hBD-2 had a statistically significant effect in relieving especially clinical symptoms on par with both dexamethasone and anti TNF-α, compounds that are commonly used to treat necrotizing enterocolitis.

### Example 5. Determining and Assessing the Efficacy of Prophylactic Treatment with Intranasal Versus Oral Mammalian β-Defensins in a Murine House Dust Mite Driven Model of Allergic Asthma

Materials and Methods:

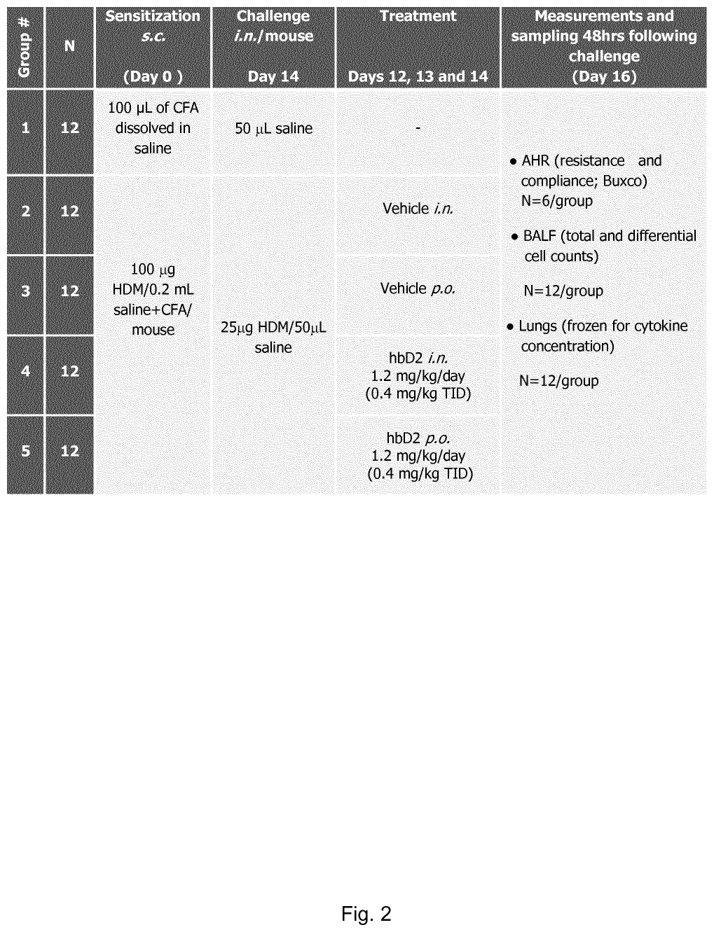

The study design is shown in FIG. 2.

Treatment Regime:

Female 7-10 weeks old BALB/c mice were randomly allocated into 5 study groups one day prior to study start and subcutaneously (SC) sensitized to house dust mite (100 μg HDM in 200 μL saline plus Freund's complete adjuvant in 0.9% saline). The mice were treated with hBD-2 orally and intranasally respectively at a dose of 1.2 mg/kg/day (0.4 mg/kg TID) starting on day 12 in the morning and continued TID at approximately 6 hours intervals. The last dose was administered on day 14 one hour prior to challenge. The total number of doses was 8 doses or a total of 2 mg/kg hBD-2. Mice were then intranasally (IN) challenged with HDM on day 14 (HDM 25 μg in 50 μL of saline) (FIG. 2).

Tests:

Airway inflammation: At 48 hours post challenge, bronchoalveolar lavage was performed washing the lungs with 3 volumes of cold PBS (0.4; 0.3 and 0.3 mL, total 1 mL). Total and differential leucocyte cell counts were determined on an automated haematological analyser Sysmex XT-2000iV.

Lung function: Starting 48 hours after HDM challenge, measurements of lung resistance and lung compliance were carried out after methacholine challenge (3.125 MCH1; 6.25 MCH2; 12.5 MCH3 and 25 mg/mL MCH4) by anaesthetized, cannulated mice using DSI's Buxco Finepoint RC system. Data are represented as airway resistance at 10 mg/kg methacholine and as dose responsive curves.

Lung sampling for cytokine analysis: After completion of every BAL, lungs were removed from the thorax, snap frozen in liquid nitrogen and stored frozen at −80 degrees Celcius until analysis of cytokine concentration of TNF-α, IL-4, IL-5, IL-6, IL-9, IL-13 and IL-33 in lung homogenate by ELISA.

Results:

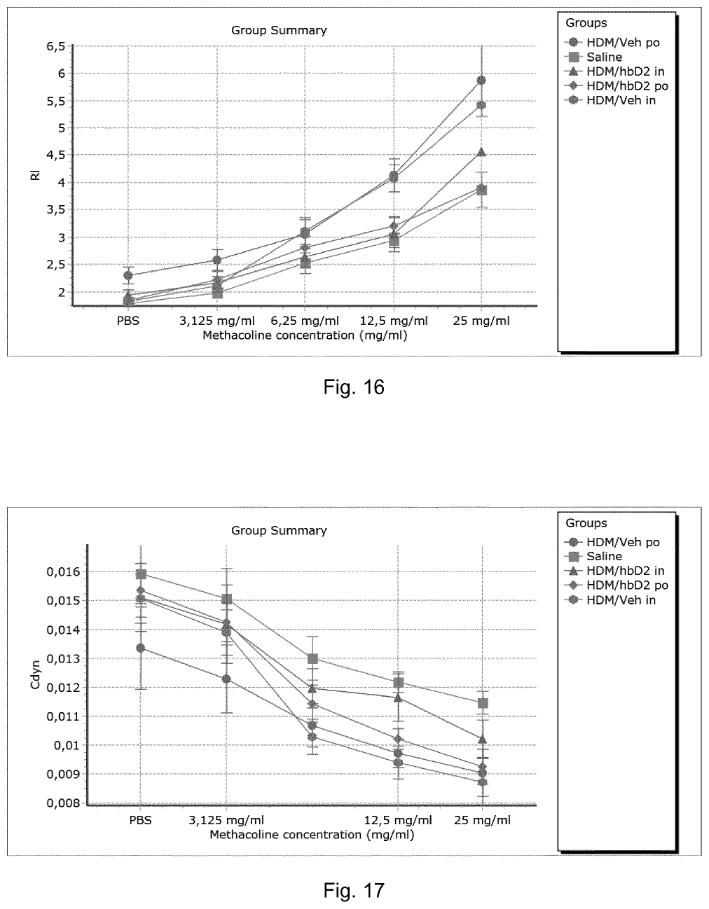

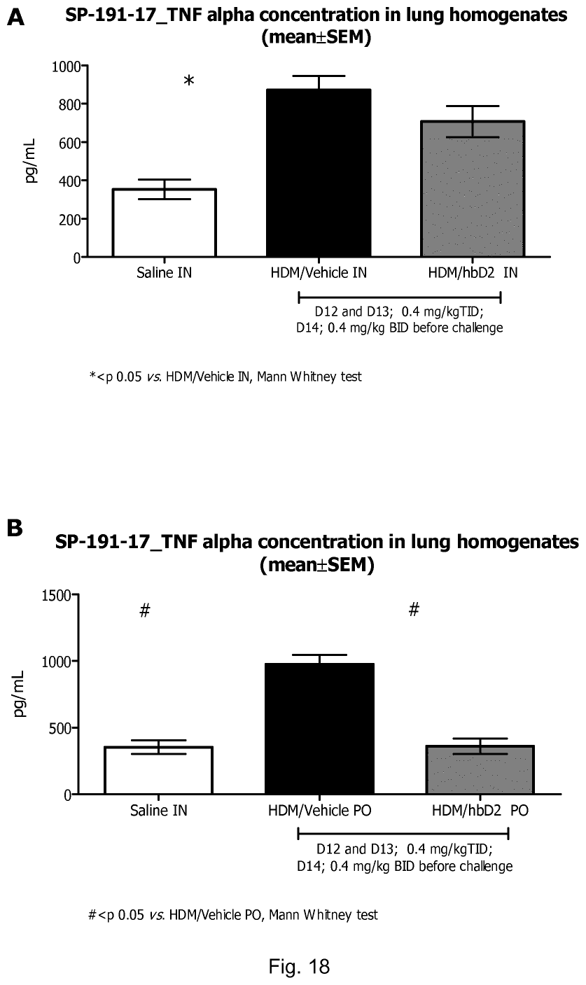

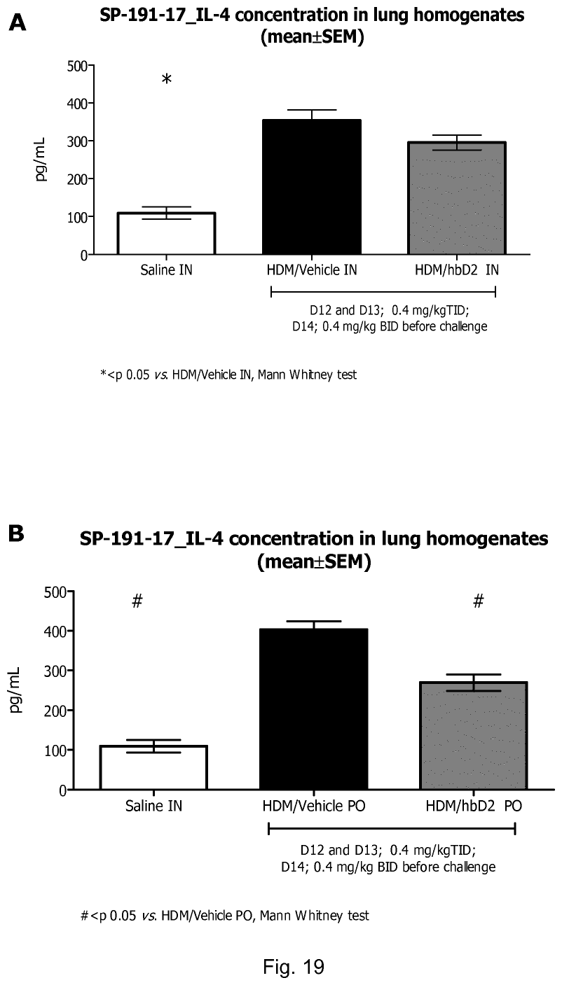

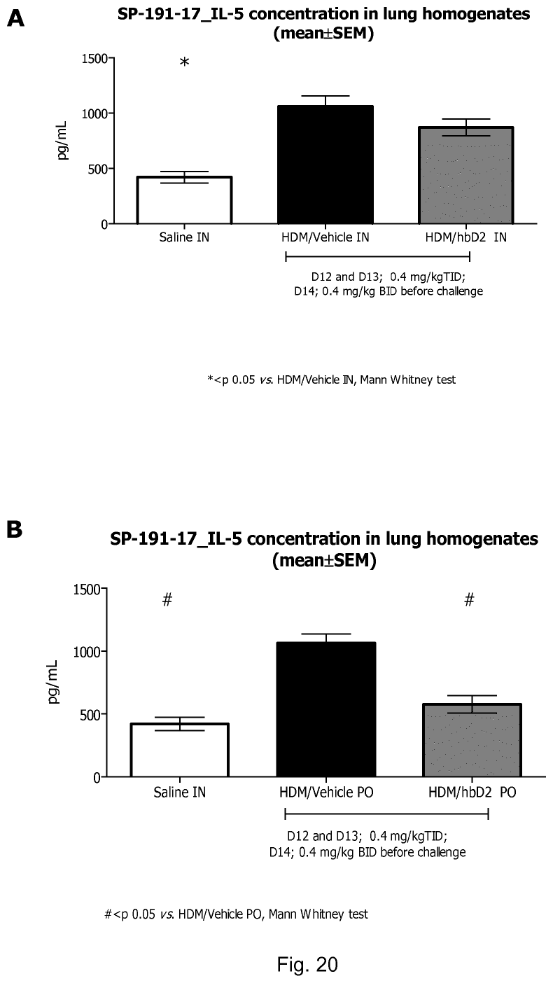

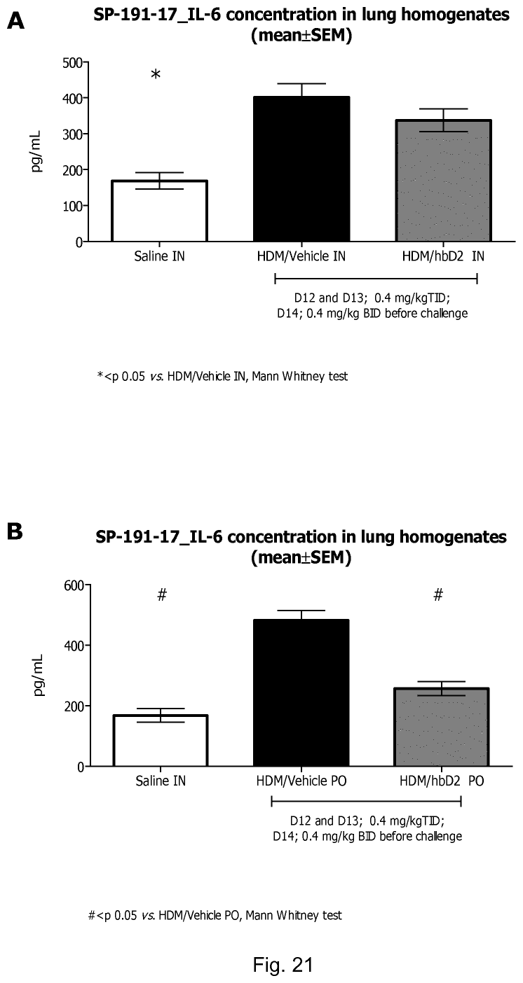

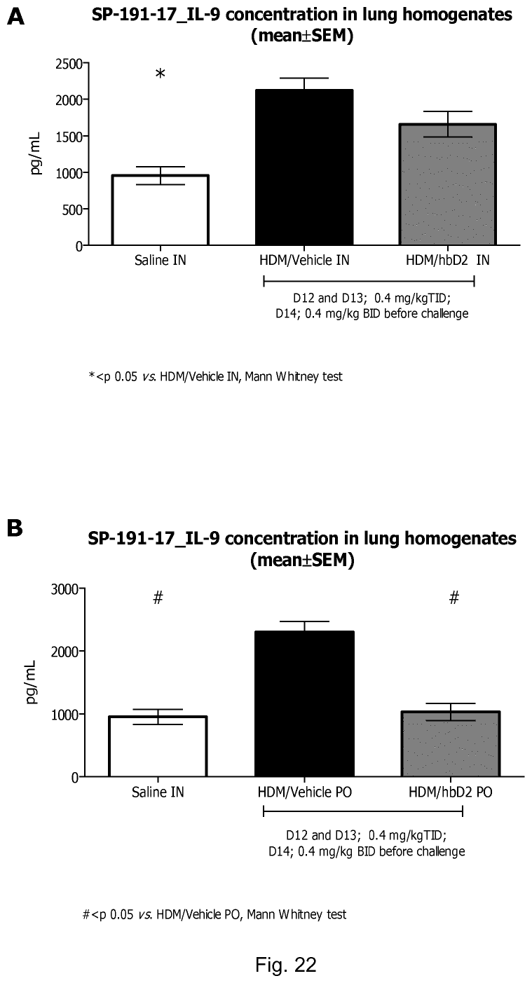

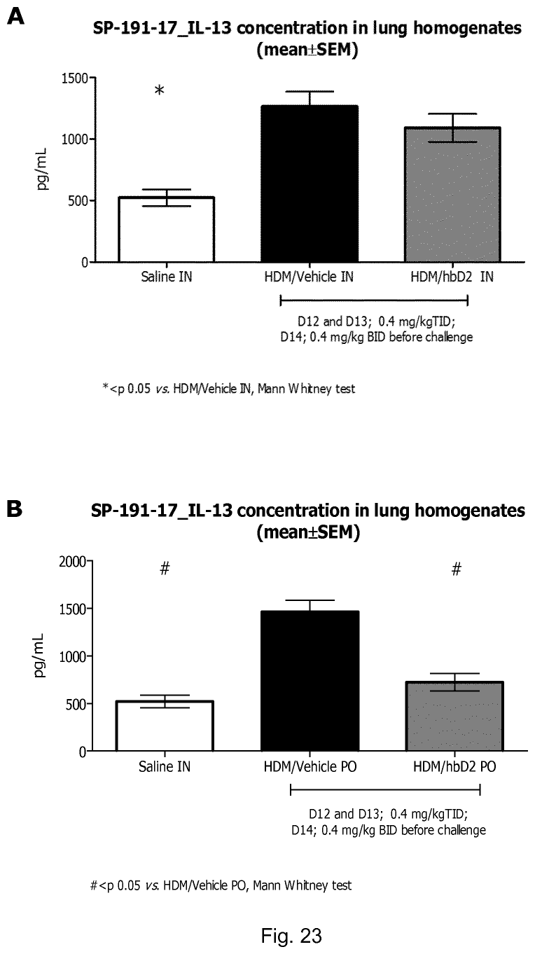

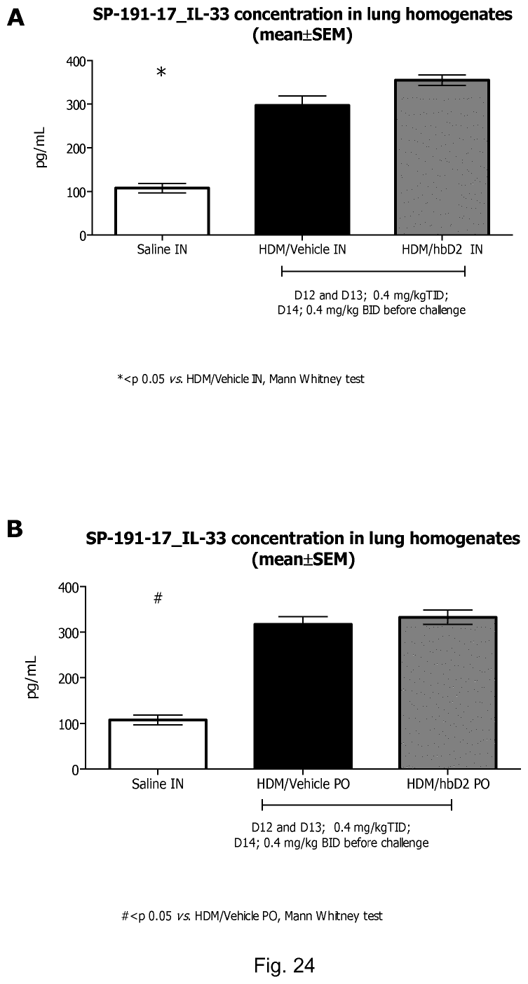

An increase of lung resistance values and decrease of pulmonary compliance values in HDM-challenged vehicle treated animals in comparison to saline-challenged (non-asthmatic) mice was observed (FIGS. 16 and 17). An inflammatory response in both vehicle-treated groups of mice (oral and intranasal) was induced by a single HDM challenge 14 days post sensitization with HDM. It was characterized by a statistically significant increase in total cell, eosinophil, neutrophil, macrophage and lymphocyte counts in BALF (p<0.05) when compared to saline-challenged controls (data not shown). Also, analysis of concentration of seven cytokines TNF-α, IL-4, IL-5, IL-6, IL-9, IL-13 and IL-33 in lung tissue homogenates revealed significantly higher levels in HDM-challenged animals compared to saline-challenged controls (FIGS. 18-24). hBD-2, both after oral and intranasal application TID, administered from day 12 to day 14 (a total of 2.0 mg/kg in 8 administrations), effectively inhibited increase of airway resistance (FIG. 16) and decrease of pulmonary compliance (FIG. 17) as compared to HDM challenged vehicle treated animals. An effect on cellular influx in BALF was observed after oral application, which significantly inhibited neutrophil counts (data not shown). A rebalance of the immune system with a complete normalization of cytokine concentrations in lung tissue homogenates was observed on TNF-α, IL-4, L-5, IL-6, IL-9 and IL-13 cytokine levels following oral administration (FIG. 18b-24b). There was a trend towards lowering of TNF-α, IL-4, IL-5, IL-6, IL-9 and IL-13 following intranasally administered hBD-2, but this was not statistically significantly different from the control (FIG. 18a-24a). All obtained results indicate clear preventive and anti-inflammatory effects of hBD-2 in the house dust mite driven mouse model of allergic asthma.

Conclusions:

The high mortality in especially ELBW infants is primarily caused by sepsis either due to bacterial translocation from necrotizing enterocolitis in the immature intestines or bacterial translocation from pneumonia and inflammation of the immature lung. The lung in especially ELBW infants is not fully developed and production of hBD-2 at best limited. This example serves to demonstrate that oral treatment with hBD-2 has a profound influence on inflammation and cytokine production in the lung and perhaps even more importantly on lung function.

### Example 6. Determining and Assessing the Efficacy of Intranasal Versus Oral Therapeutic Intervention with Mammalian β-Defensins in a Murine House Dust Mite/Freunds Complete Adjuvant Driven Model of Allergic Asthma

Materials and Methods:

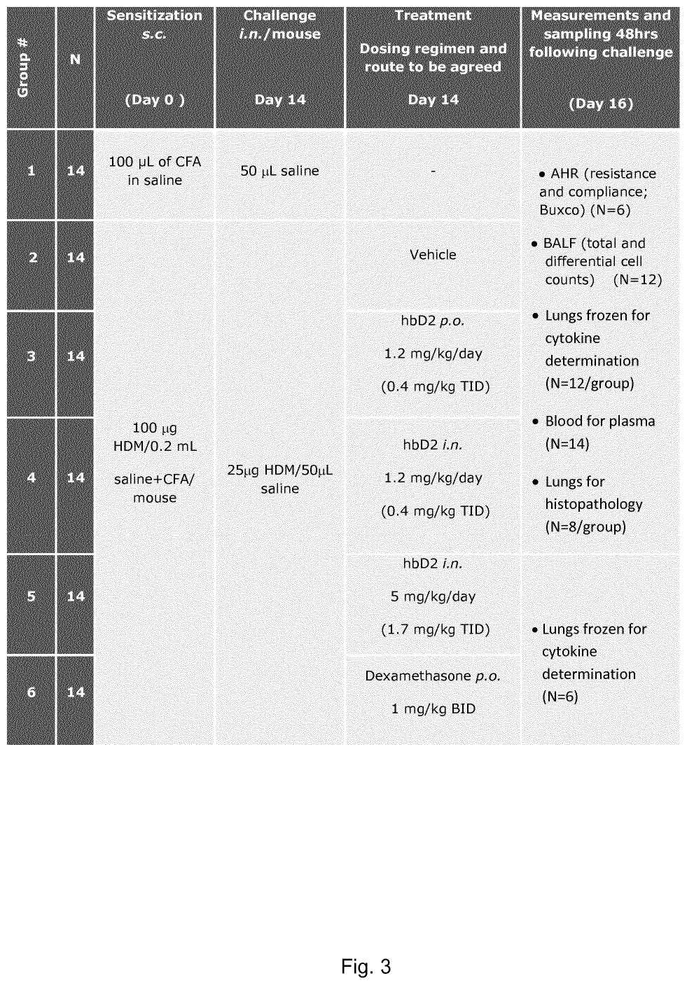

The study design is shown in FIG. 3.

Treatment Regime:

Female 7-10 weeks old BALB/c mice were randomly allocated into 6 study groups one day prior to study start and subcutaneously (SC) sensitized to house dust mite (100 μg HDM in 200 μL saline plus Freund's complete adjuvant in 0.9% saline). Mice were then intranasally (IN) challenged with HDM on day 14 (HDM 25 μg in 50 μL of saline). Dexamethasone was administered orally (1 mg/kg BID; 50 μL phosphate buffered saline (PBS)) on day 14. hBD-2 was administered IN or orally (1.7 mg/kg TID IN; 0.4 mg/kg TID IN; 0.4 mg/kg TID orally, 50 μL phosphate buffered saline) on day 14. The initial dose was administered 60 minutes prior to challenge, and the subsequent two doses approximately 6 hours apart (FIG. 3).

Tests:

Airway inflammation: At 48 hours post challenge, bronchoalveolar lavage was performed washing the lungs with 3 volumes of cold PBS (0.4; 0.3 and 0.3 mL, total 1 mL). Total and differential leucocyte cell counts were determined on an automated haematological analyser Sysmex XT-2000iV.

Lung function: Starting 48 hours after HDM challenge, measurements of lung resistance and lung compliance were carried out after methacholine challenge (3.125 MCH1; 6.25 MCH2; 12.5 MCH3 and 25 mg/mL MCH4) by anaesthetized, cannulated mice using DSI's Buxco Finepoint RC system. Data are represented as airway resistance at 10 mg/kg methacholine and as dose responsive curves.

Lung sampling for cytokine analysis: After completion of every BAL, lungs were removed from the thorax, snap frozen in liquid nitrogen and stored frozen at −80 degrees Celcius until analysis of cytokine concentration of IL-1β, TNF-α, IL-6, IL-10 and IFNγ by ELISA.

Results:

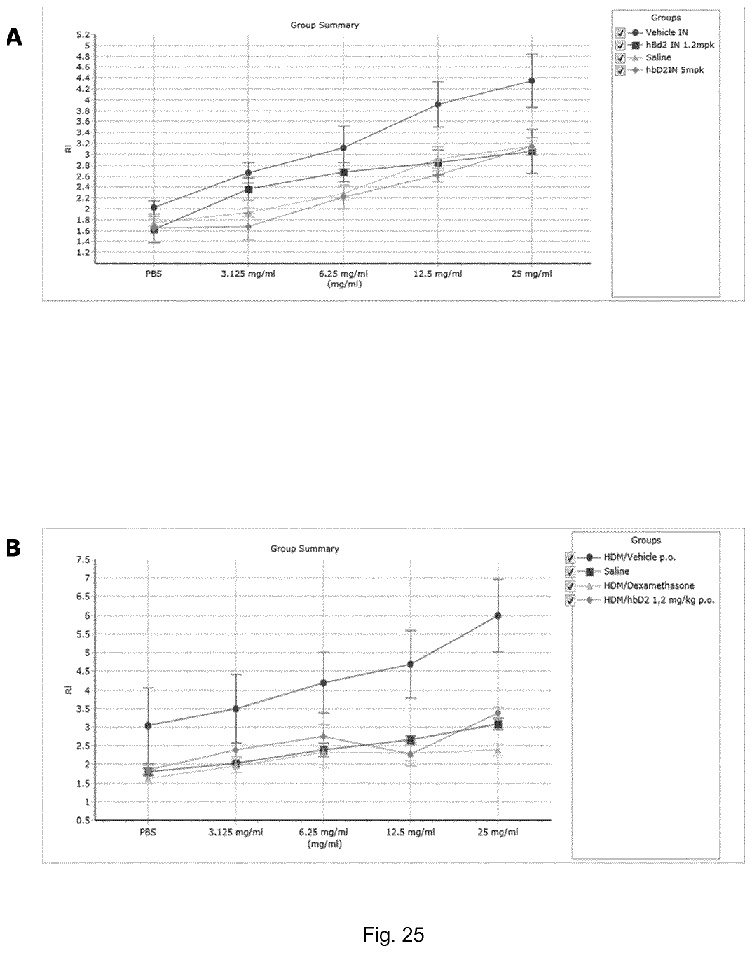

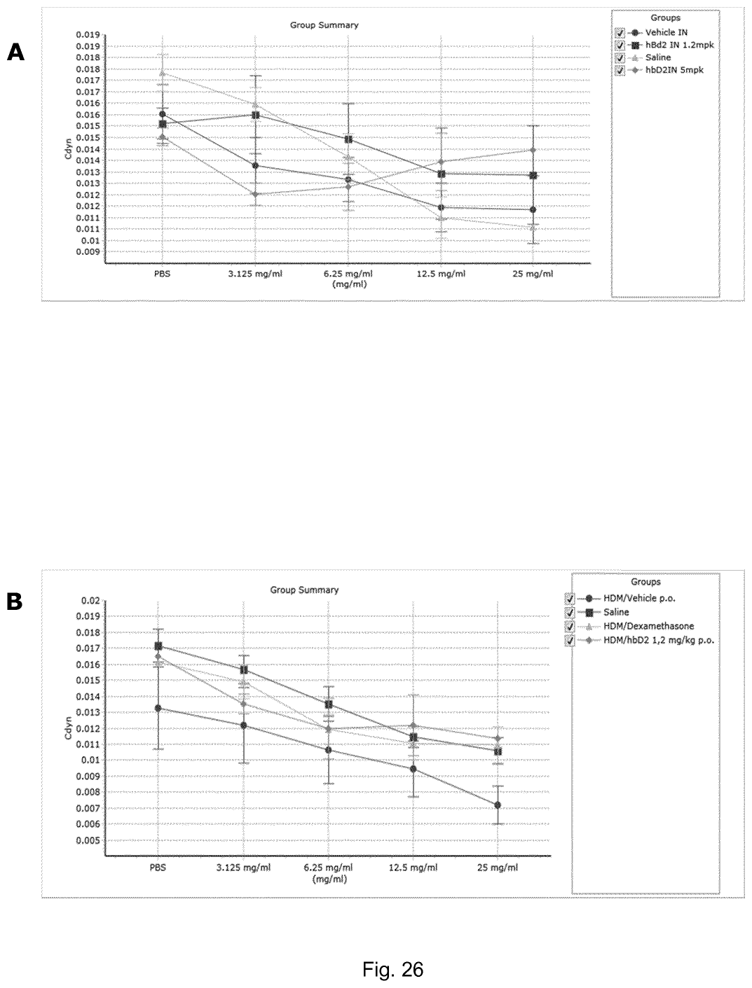

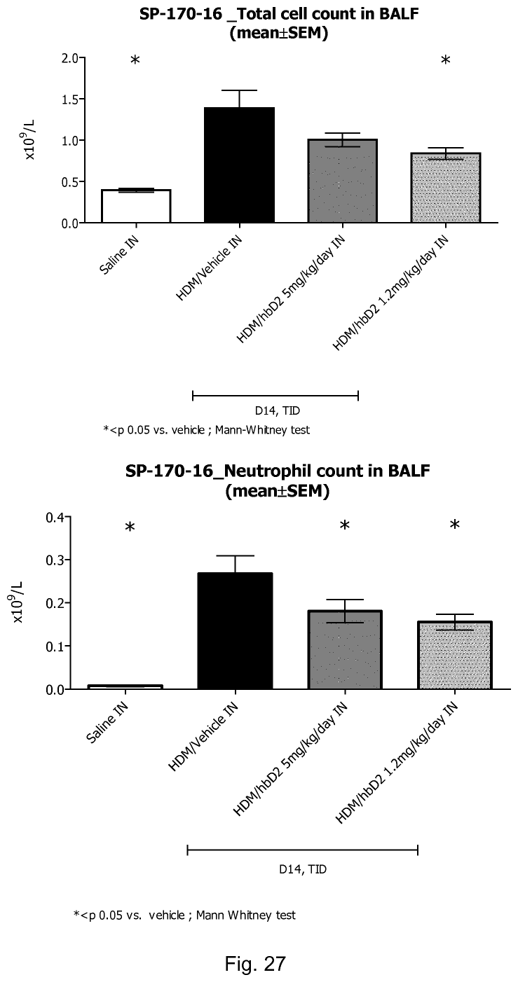

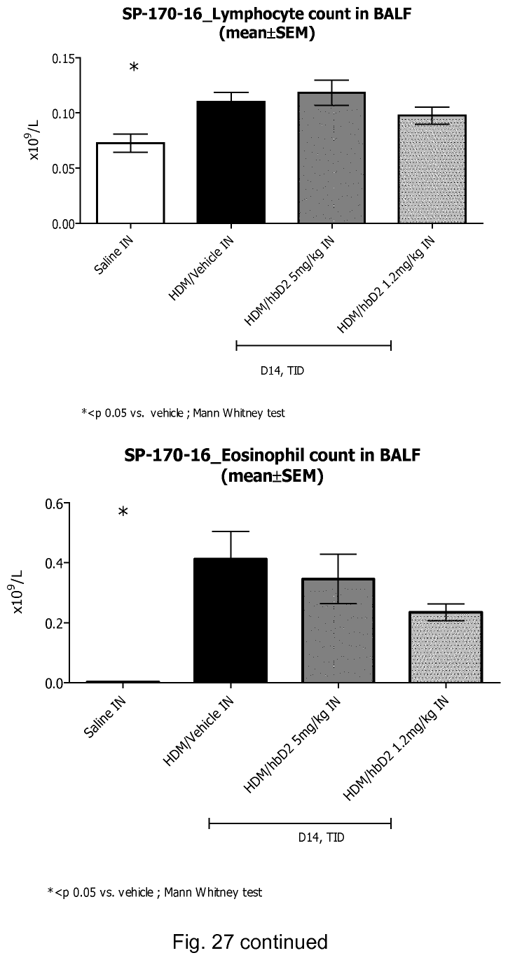

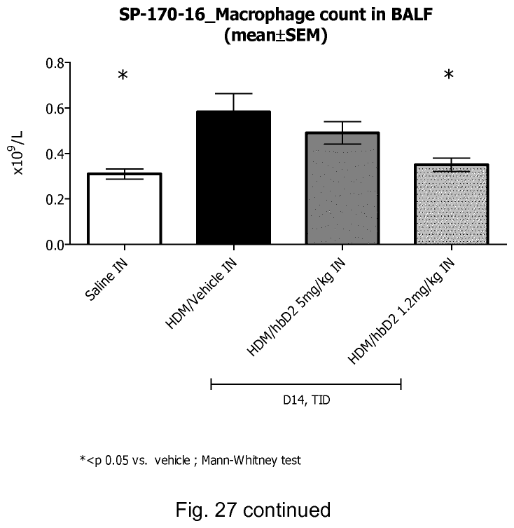

An increase of lung resistance values and decrease of pulmonary compliance values in HDM-challenged vehicle treated animals in comparison to saline-challenged (non-asthmatic) mice was observed (FIGS. 25 and 26). An inflammatory response in both vehicle-treated groups of mice (oral and intranasal) was induced by a single HDM challenge 14 days post sensitization with HDM and adjuvant. It was characterized by a statistically significant increase in total cell, eosinophil, neutrophil, macrophage and lymphocyte counts in BALF (p<0.05) when compared to saline-challenged controls (FIG. 27). Also, analysis of concentration of five cytokines TNF-α, IL-6, IL-10 and IFN-γ in lung tissue homogenates revealed significantly higher levels in HDM-challenged animals compared to saline-challenged controls.

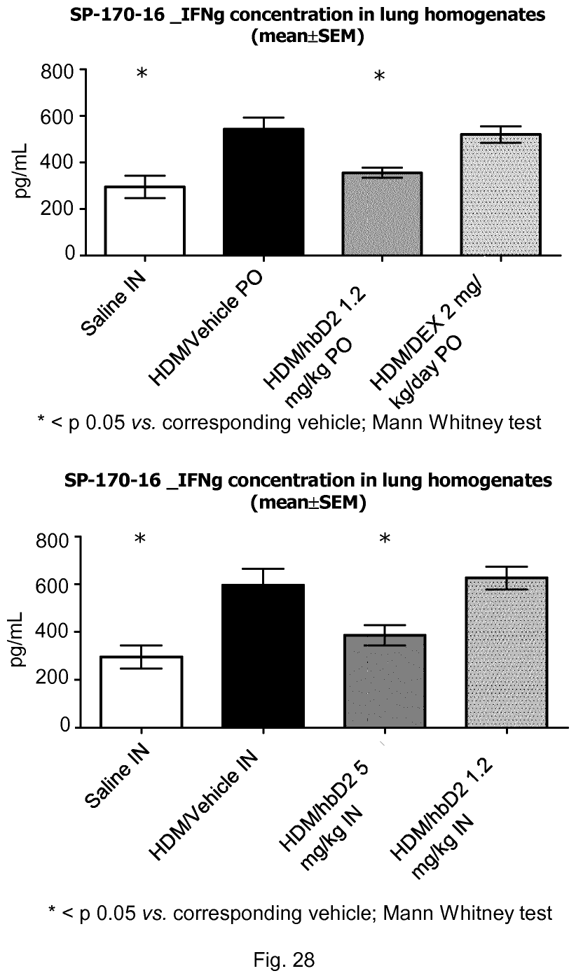

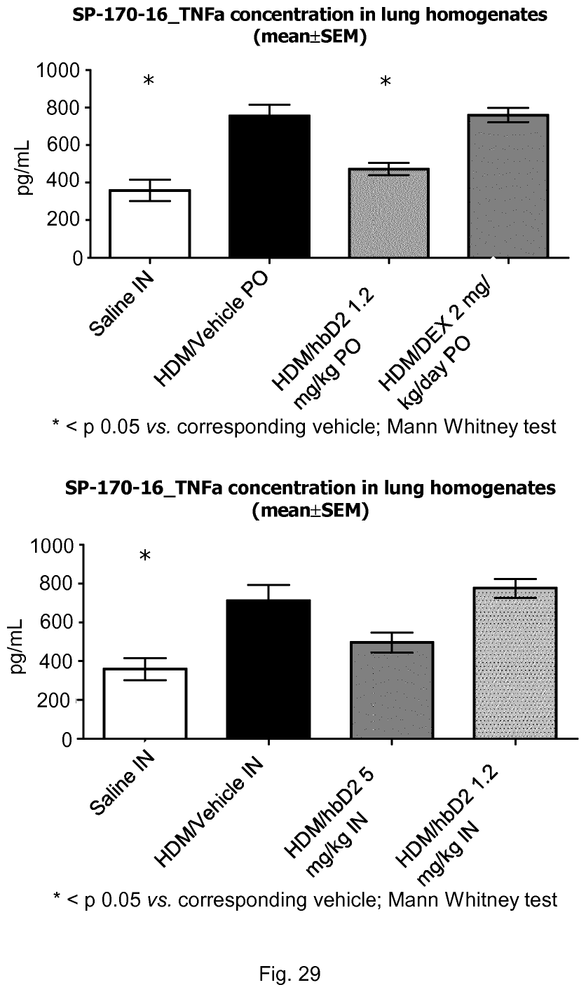

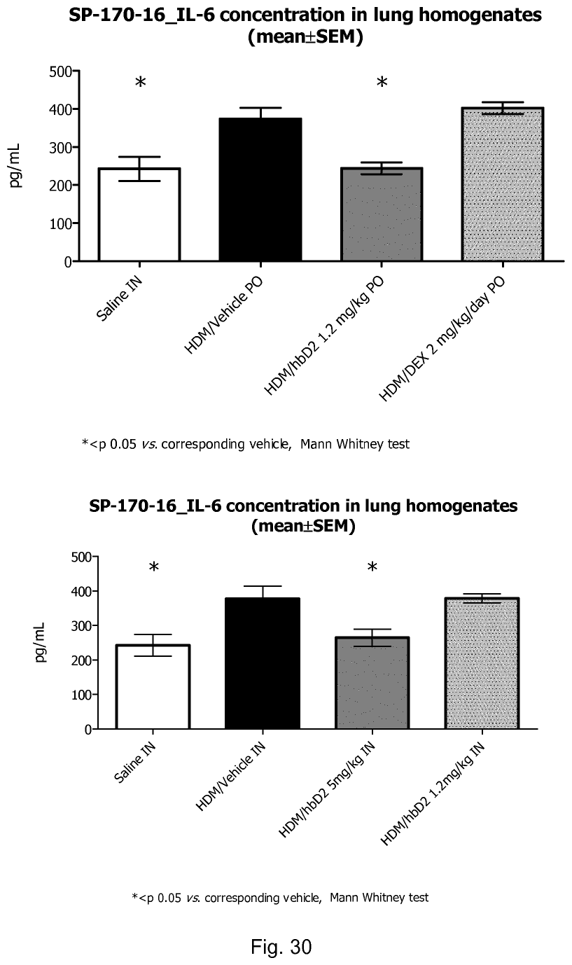

Dexamethasone treatment significantly inhibited total cell and eosinophil counts but not neutrophil, macrophage and lymphocyte counts in BALF (data not shown). In accordance with the cellular data, dexamethasone did not influence levels of TNF-α, IL-6, IL-10 and IFN-γ in lung tissue homogenates as compared to HDM/vehicle control (FIGS. 28 to 29). However, it influenced AHR measurements related to eosinophil counts. Obtained results indicate that this model is steroid resistant to a certain degree. hBD-2, both after oral and intranasal application TID, on day 14, effectively inhibited increase of airway resistance (FIGS. 25a and 25b) and decrease of pulmonary compliance (FIGS. 26a and 26b) as compared to HDM challenged vehicle treated animals. A more prominent effect was observed on some measured parameters after intranasal application, such as cellular influx in BALF, where both doses (0.4 mg/kg/day TID and 1.7 mg/kg/day TID) significantly inhibited total, neutrophil and macrophage cell counts and a trend towards lowering of eosinophils (FIG. 27), while the steroid standard dexamethasone failed to inhibit them (data not shown). Similar significant effects were observed on IL-6, IL-10 and IFN-γ cytokine levels in lung tissue homogenates with both dosing routes (FIG. 28, 32, 35). Perorally administered hBD-2 significantly reduced TNF-α (FIG. 29), while the intranasally administered hBD-2 was not significantly different from the controlAll obtained results indicate clear anti-inflammatory effects of hBD-2 in the house dust mite/Freund's complete adjuvant driven mouse model of allergic asthma.

Conclusions:

The high mortality in especially ELBW infants is primarily caused by sepsis either due to bacterial translocation from necrotizing enterocolitis in the immature intestines or bacterial translocation from pneumonia and inflammation of the immature lung. The lung in especially ELBW infants is not fully developed and production of hBD-2 at best limited. This example serves to demonstrate that oral treatment with hBD-2 has a profound influence on inflammation and cytokine production in the lung and perhaps even more importantly on lung function.

### Example 7. To Determine and Assess the Efficacy of Intranasal Versus Oral Therapeutic Intervention with Administration of Mammalian β-Defensins in a Murine House Dust Mite/Freund's Complete Adjuvant Driven Model of Allergic Asthma

Materials and Methods:

Study design is shown in FIG. 3.

Treatment regime:

Female 7-10 weeks old BALB/c mice randomly allocated into 4 study groups one day prior to study start were subcutaneously (SC) sensitized to house dust mite (100 μg HDM in 200 μL saline plus Freund's complete adjuvant in 0.9% saline). Mice were intranasally (IN) challenged with HDM on day 14 (HDM 25 μg in 50 μL of saline). hBD-2 was administered IN or orally (0.4 mg/kg TID IN; 0.4 mg/kg TID orally, 50 μL phosphate buffered saline) on day 14. The initial dose was administered 60 minutes prior to challenge, and the subsequent doses approximately 6 hours apart (FIG. 3, groups 1-4)).

Tests:

Blood sampling: All terminal blood samples were collected via jugular vein bleeds. Blood was sampled to Li-heparin tubes, put on ice and immediately centrifuged at 4° C. Plasma was separated and stored at −80° C. until the potential SOFA analysis. Lung tissue sampling: The lungs were exposed and excised by gently opening the thorax and by cutting down either side of the sternum and ribs and trimming back. Lungs from the first 6 animals per group were removed from thorax, snap frozen in liquid nitrogen and stored frozen at −80° C. until analysis of cytokine concentration of IL-4, IL-5, IL-8 (KC), IL-9 and IL-13 in lung homogenate by ELISA.

Lungs from the other 8 animals per group were inflated in situ with 10% buffered formalin, removed from thorax, placed individually in 10% buffered formalin, paraffin embedded in toto, sectioned and H&E/PAS stained. The paraffin blocks were retained for the IHC analysis.

Read-Outs
Histopathology (H&E; PAS) (N=8/group; total N=32)
Cytokines in lung tissue homogenates (IL-4, IL-5, IL-8 (KC), IL-9 and IL-13) (N=6/group; total N=24)

Histopathology

Cellular influx (mononuclears, eosinophils, neutrophils) was assessed semi-quantitatively on H&E stained slides separately for peri-bronchial/bronchiolar and perivascular space as follows:
0 absent
1 few scattered inflammatory cells
2 larger aggregates
3 marked accumulation of cells

Overall score for inflammation was calculated as the sum of all individual scores. Goblet cell metaplasia, separately at a level of large airways and distal airways, was assessed at PAS-stained slides as follows:
0 no mucus containing cells along basement membrane
1 few positive cells along basement membrane with less than 75% of the cytoplasm stained
2 few positive cells along basement membrane with more than 75% of the cytoplasm stained
3 numerous positive cells along basement membrane with less than 75% of the cytoplasm stained
4 numerous positive cells along basement membrane with more than 75% of the cytoplasm stained

Statistical Evaluation

Data was processed using MS Excel. Statistical analysis was performed using GraphPad Prism software (version 5.04). Differences between groups are considered statistically significant when p<0.05.

Statistical analysis of selected histological score-values data was performed using median and non-parametric Mann-Whitney test.

Results:

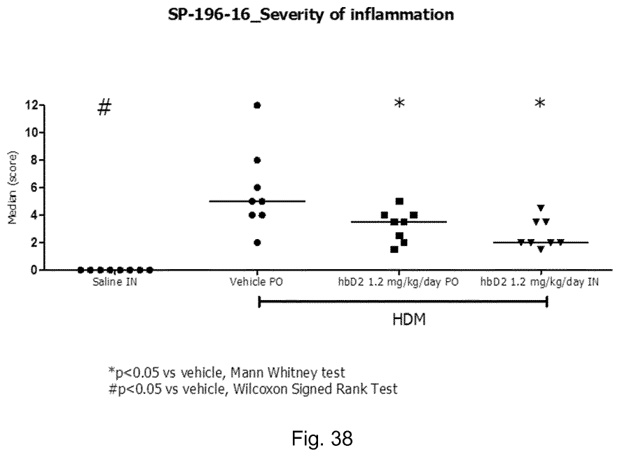

An inflammatory response in both vehicle-treated groups of mice (oral and intranasal) was induced by a single HDM challenge 14 days post sensitization with HDM and adjuvant. It was characterized by a statistically significant increase in concentration of five cytokines IL-4, IL-5, IL-8, IL-9 and IL-13 in lung tissue homogenates and by severe histological inflammatory changes of lung tissue in HDM-challenged animals compared to saline-challenged controls (FIGS. 37 and 38).

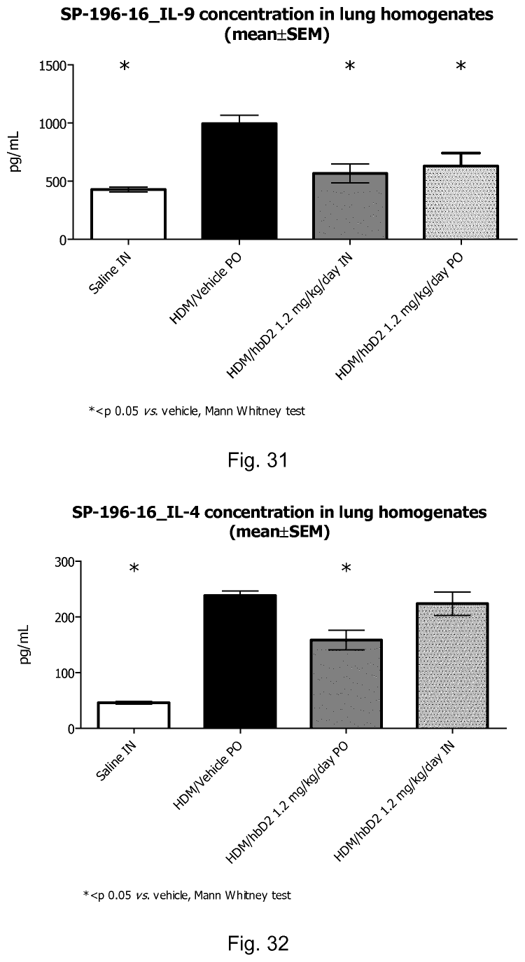

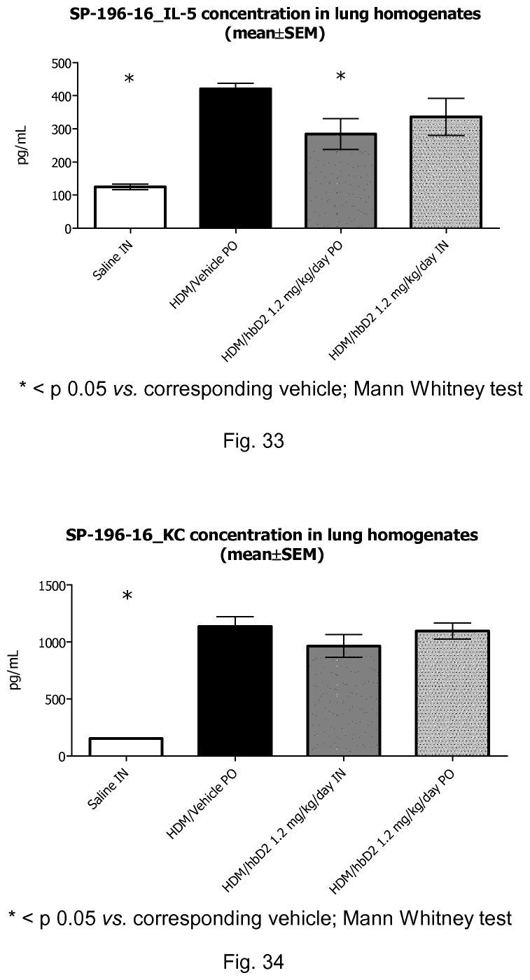

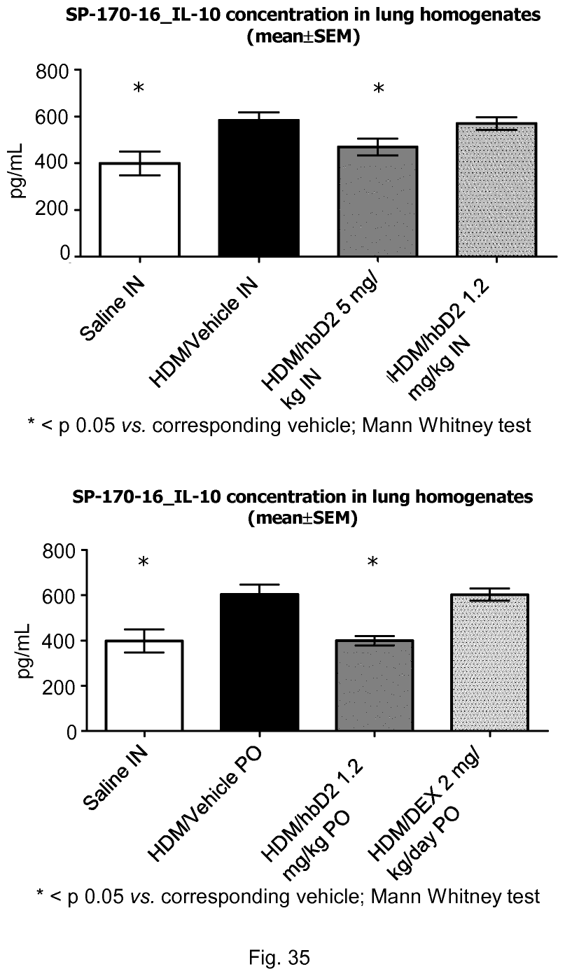

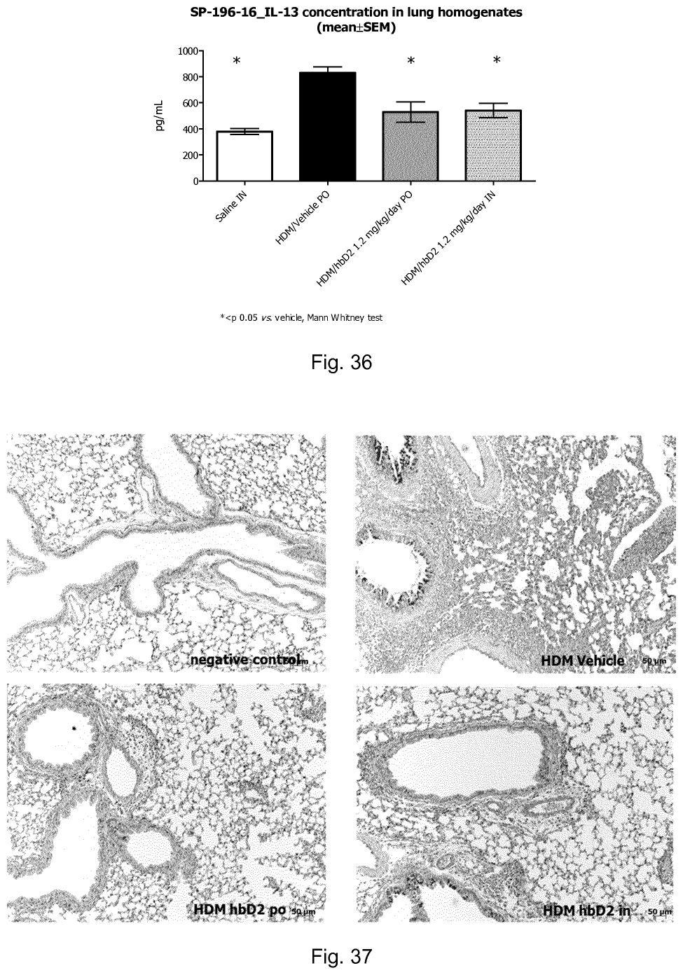

hBD-2, both after oral and intranasal application TID, on day 14, effectively inhibited the increase in histological inflammation of lung tissue as compared to HDM challenged vehicle treated animals (FIGS. 37 and 38). Significant effects were observed on IL-4, IL-5, IL-9 and IL-13 cytokine levels in lung tissue homogenates following oral administration (FIG. 32, 33, 31, 36) and on IL-9 and IL-13 (FIG. 31, 36) following IN administration. All obtained results indicate clear anti-inflammatory effects of hBD-2 in the house dust mite/Freund's complete adjuvant driven mouse model of allergic asthma.

Conclusions:

The high mortality in especially ELBW infants is primarily caused by sepsis either due to bacterial translocation from necrotizing enterocolitis in the immature intestines or bacterial translocation from pneumonia and inflammation of the immature lung. The lung in especially ELBW infants is not fully developed and production of hBD-2 at best limited. This example serves to demonstrate that oral treatment with hBD-2 has a profound influence on inflammation and cytokine production in the lung and perhaps even more importantly on lung function.

### Example 8

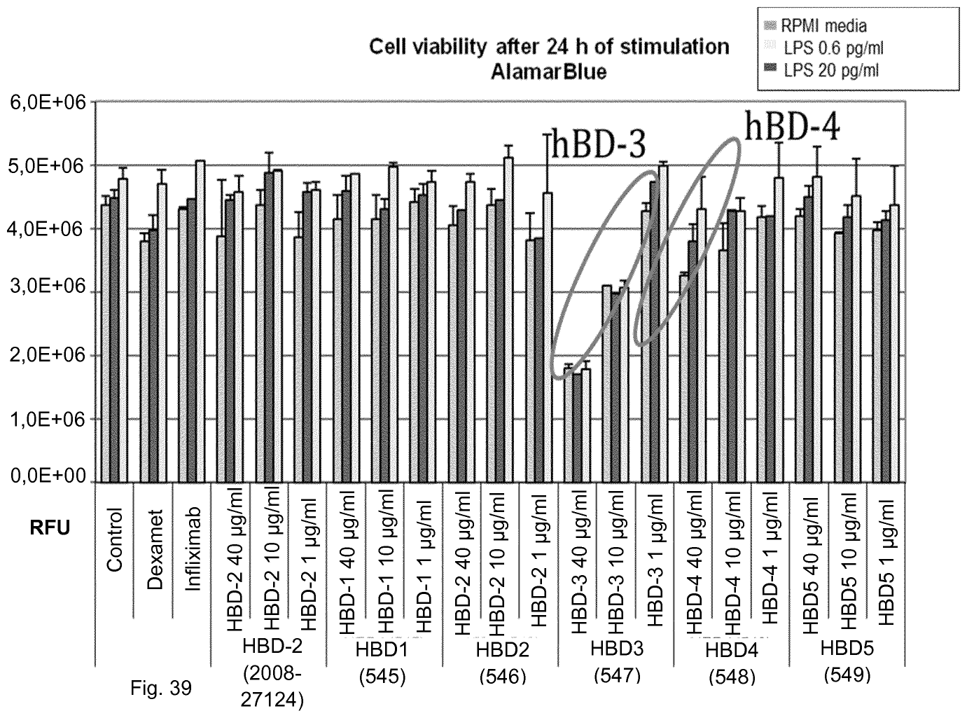

PBMC cell viability following exposure for 24 hours to Alamar Blue and HD5, hBD-1, hBD-2, hBD-3 and hBD-4 at concentrations of 1, 10 and 40 μg/ml at 0.6 and 20.0 pg/ml LPS respectively (FIG. 39).

The experiment shows that HD5, hBD-1 and hBD-2 are not toxic irrespectively of concentration whereas hBD-4 shows moderately increasing toxicity and hBD-3 shows profoundly increasing toxicity at increasing concentrations.

### Example 9

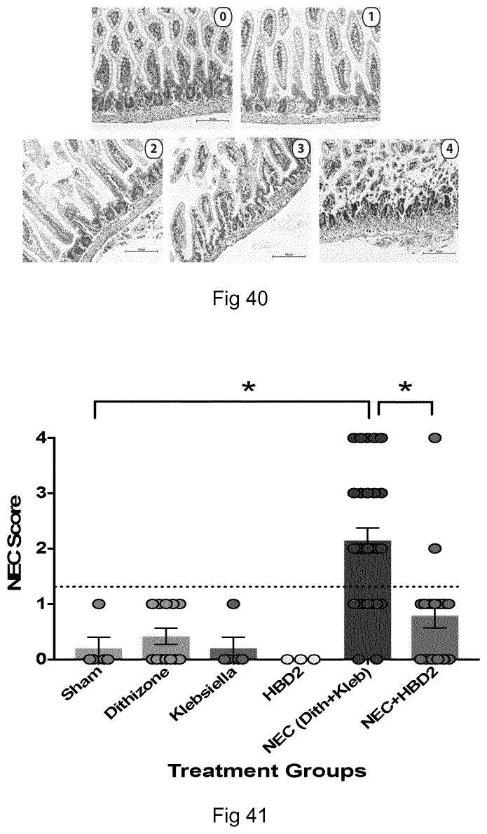

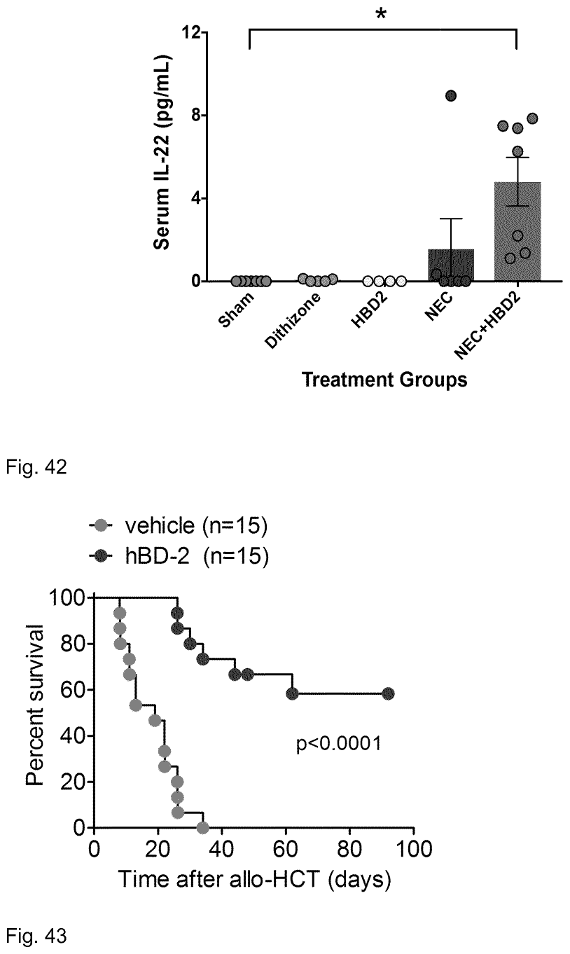

Incidence of necrotizing enterocolitis and IL-22 concentration in a murine model of necrotizing enterocolitis. Intestinal mucosal injury consistent with human necrotizing enterocolitis was induced by a combination of Dithizone followed by Klebsiella pneumonia challenge by oral gavage in postnatal day 14 C57BL/6J mice. The severity of necrotizing enterocolitis was assessed by a histological scoring system with a score of >2 indicating significant damage (FIG. 40). Neither Dithizone nor K. pneumonia challenge induced mucosal injury by themselves, but the combination of Dithizone followed by K. pneumonia challenge induced severe and statistically significant mucosal damage measured 16 hours post challenge (FIG. 41). Prophylactic oral treatment with 1.2 mg/kg hBD-2 one hour post K. pneumonia challenge dramatically and statistically significantly reduced the incidence of necrotizing enterocolitis to normal (FIG. 41). The concentration of IL-22, a mucosal barrier enhancing cytokine, was statistically significantly increased 9 hours post challenge.

### Example 10

Sequences

| SEQ | | |
|---|---|---|
| ID | | |
| NO: | Name | Sequence |
| 1 | hBD-1 | DHYNCVSSGGQCLYSACPIFTKIQGTCYRGKAKCCK |
| 2 | hBD-2 | GIGDPVTCLKSGAICHPVFCPRRYKQIGTCGLPGTKCCKKP |
| 3 | hBD-3 | GIINTLQKYYCRVRGGRCAVLSCLPKEEQIGKCSTRGRKCCRRK |
| 4 | hBD-4 | ELDRICGYGTARCRKKCRSQEYRIGRCPNTYACCLRK |
| 5 | HD5 | ATCYCRTGRCATRESLSGVCEISGRLYRLCCR |
| 6 | HD6 | AFTCHCRRSCYSTEYSYGTCTVMGINHRFCCL |
| 7 | Mouse beta | KINNPVSCLRKGGRCWNRCIGNTRQIGSCGVPFLKCCKRK |
| | defensin 3 | |
| 8 | HNP-1 | ACYCRIPACIAGERRYGTCIYQGRLWAFCC |
| 9 | HNP-2 | CYCRIPACIAGERRYGTCIYQGRLWAFCC |
| 10 | HNP-3 | DCYCRIPACIAGERRYGTCIYQGRLWAFCC |
| 11 | HNP-4 | VCSCRLVFCRRTELRVGNCLIGGVSFTYCCTRV |
| 12 | Human | LLGDFFRKSKEKIGKEFKRIVQRIKDFLRNLVPRTES |
| | Cathelicidin | |
| | (LL-37) | |
| 13 | Human | GRRRSVQWCAVSQPEATKCFQWQRNMRKVRGPPVSCIKRDSP |
| | Lactoferrin | IQCIQAIAENRADAVTLDGGFIYEAGLAPYKLRPVAAEVYGTERQ |
| | | PRTHYYAVAVVKKGGSFQLNELQGLKSCHTGLRRTAGWNVPIG |
| | | TLRPFLNWTGPPEPIEAAVARFFSASCVPGADKGQFPNLCRLCA |
| | | GTGENCAFSSQEPYFSYSGAFKCLRDGAGDVAFIRESTVFEDLS |
| | | DEAERDEYELLCPDNTRKPVDKFKDCHLARVPSHAVVARSVNG |
| | | KEDAIWNLLRQAQEKFGKDKSPKFQLFGSPSGQKDLLFKDSAIG |
| | | FSRVPPRIDSGLYLGSGYFTAIQNLRKSEEEVAARRARVVWCAV |
| | | GEQELRKCNQWSGLSEGSVTCSSASTTEDCIALVLKGEADAMS |
| | | LDGGYVYTAGKCGLVPVLAENYKSQQSSDPDPNCVDRPVEGYL |
| | | AVAVVRRSDTSLTWNSVKGKKSCHTAVDRTAGWNIPMGLLFNQ |
| | | TGSCKFDEYFSQSCAPGSDPRSNLCALCIGDEQGENKCVPNSN |
| | | ERYYGYTGAFRCLAENAGDVAFVKDVTVLQNTDGNNNEAWAK |
| | | DLKLADFALLCLDGKRKPVTEARSCHLAMAPNHAVVSRMDKVE |
| | | RLKQVLLHQQAKFGRNGSDCPDKFCLFQSETKNLLFNDNTECLA |
| | | RLHGKTTYEKYLGPQYVAGITNLKKCSTSPLLEACEFLRK |
| 14 | Lactoferricin-H | GRRRSVQWCAVSQPEATKCFQWQRNMRKVRGPPVSCIKRDSP |
| | | IQCIQA |
| 15 | Human Lysozyme | KVFERCELARTLKRLGMDGYRGISLANWMCLAKWESGYNTRAT |
| | | NYNAGDRSTDYGIFQINSRYWCNDGKTPGAVNACHLSCSALLQ |
| | | DNIADAVACAKRVVRDPQGIRAWVAWRNRCQNRDVRQYVQGC |
| | | GV |
| 16 | truncated hBD2 | PVTCLKSGAICHPVFCPRRYKQIGTCGLPGTKCCKKP |

### Example 11

To determine and assess the efficacy of prophylactic treatment with oral hBD-2 in a murine model of acute graft versus host disease following stem cell transplantation.

Materials and Methods

Treatment Regime:

12 female BALB/c mice were irradiated with 4.5 Gy (2×498 sec) at intervals of at least 4 hours on day 0. Bone marrow was harvested: hind limbs harvested from 2 female WT C57BL/6 mice aseptically. Skeletal muscle was removed and epiphysis cut away. Lumen of bone flushed with PBS and cells pelleted and taken up. Harvest of T-cells: spleens from 2 female WT C57BL/6 mice were meshed through a 100 μM cell strainer into a PBS filled dish. Cells were taken up in PBS and transferred into a 50 mL falcon tube and spun down. Cells were taken up in 1 mL PBS and 20 μL of CD4 microbead+20 μL CD8 microbeads per spleen added for incubation for 20 min at 4 degrees C. Cells were washed with PBS and applied to MACS separation in order to positively select CD4+ and CD8+ cells. Positively selected T-cells were counted and taken up. Transplantation: directly after the second irradiation, all WT BALB/c recipient mice were injected with 5×1.000.000 BM cells (50 μL) i.v. into the retroorbital venous plexus under isoflurane anesthesia. 15 BALB/c mice were treated with 1.2 mg of hBD2/kg BW/day in 100 μL PBS per day vial oral gavage from day 0-10.

15 BALB/c mice received vehicle 100 μL PBS per day via oral gavage from day 0-10.

Tests:

The mice were weighed daily for the initial 7 days and survival was monitored for 100 days.

Results

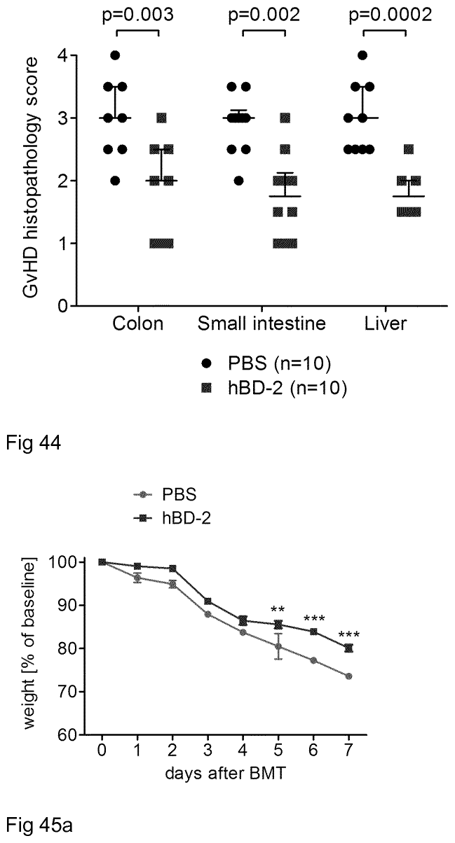

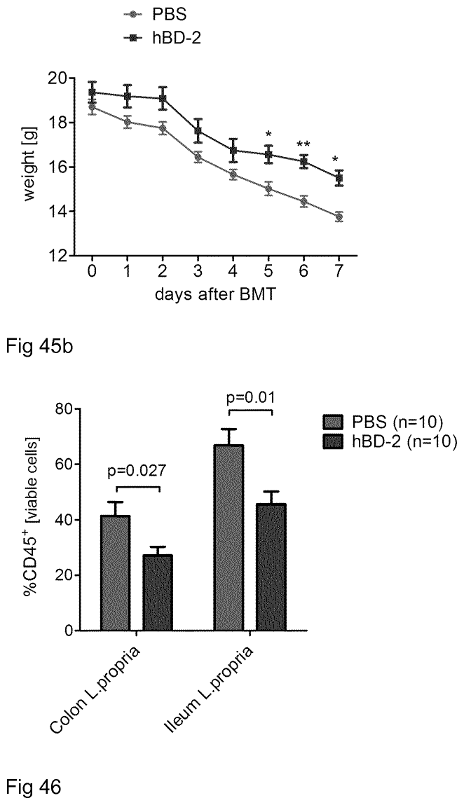

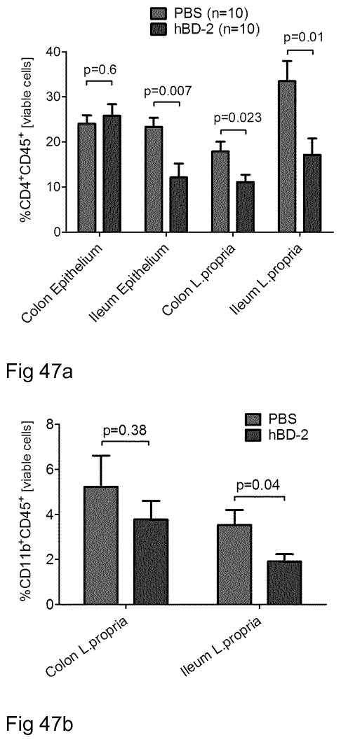

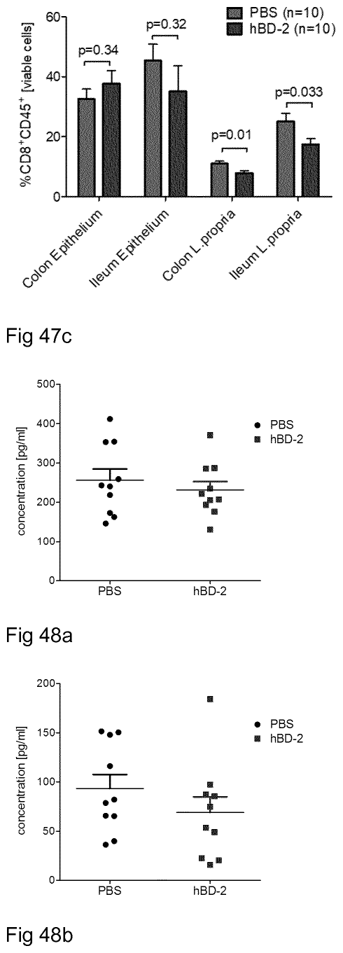

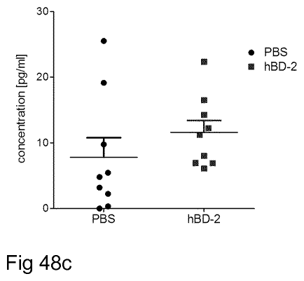

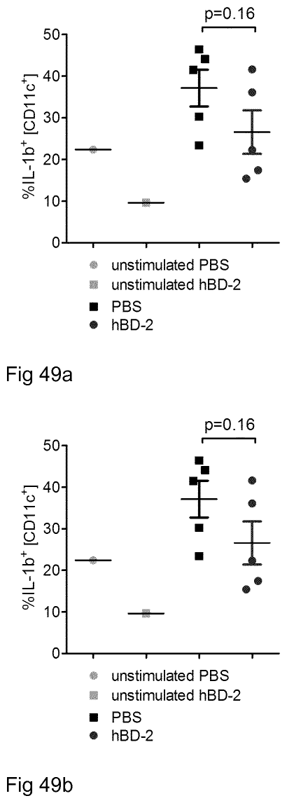

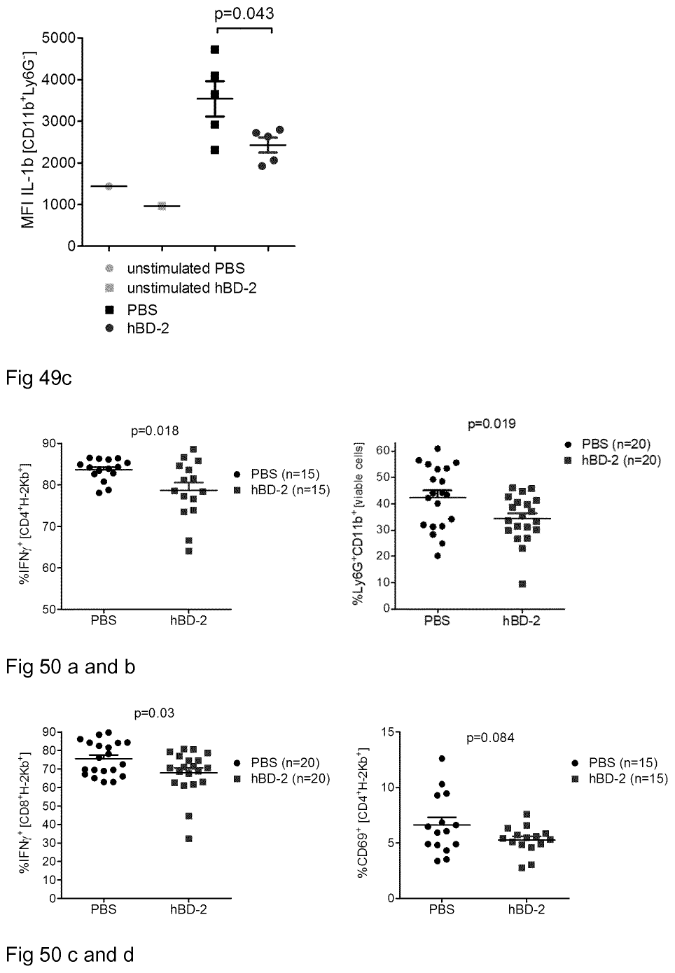

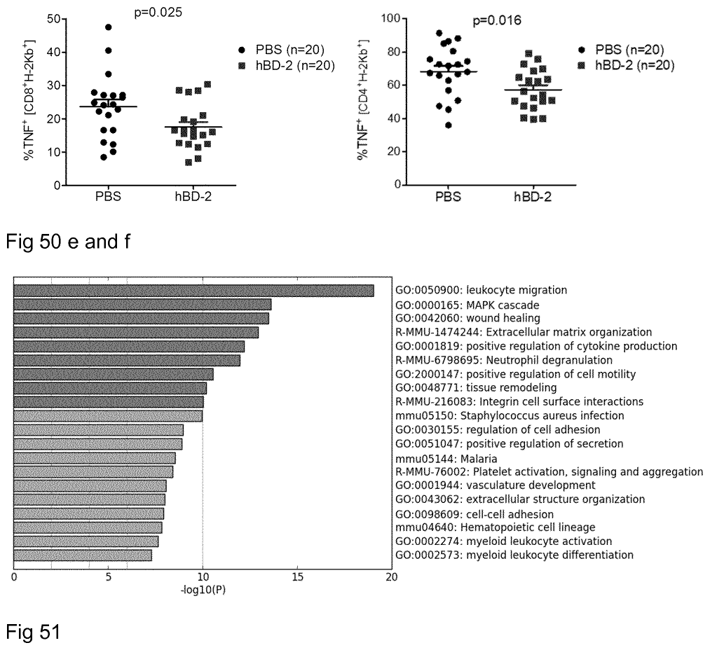

All 15 mice in the vehicle group had died by day 35 whereas only 4 or less than 30% of the hBD2 treated mice had died by day 35 and 8 mice were still alive by day 100 p<0.0001 (FIG. 43). The histology score of the small intestine, colon and liver were all highly statistically lower for the hBD-2 treated group compared with PBS (FIG. 44). The hBD2 treated mice lost statistically significantly less weight the first 7 days following bone marrow transplantation suggesting improved gut health and gut integrity (FIG. 45). Treatment with hBD2 reduced the infiltration with CD45+ leucocytes (FACS analysis in FIG. 46); intestinal T cell and myeloid cell infiltration (FIG. 47 a-c) in lamina propria of the colon. Prophylactic treatment with hBD2 also showed reduction of TNF-α and IL-6 concentrations and increased concentration of IL-10 (FIG. 48 a-c). The hBD-2 treatment additionally showed a reduction of IL-1β from myeloid cells (FACS analysis of the spleen in FIG. 49 a-c). FACS analysis of the spleen also showed reduced number of neutrophils (FIG. 50a); reduced Th1 cytokine production of especially TNF-α and IFN-γ (FIG. 50 b-f). Microarray analysis of the colon samples showed decreased inflammation, leucocyte and myeloid cell migration and tissue remodelling in the hBD-2 treated group versus PBS (FIG. 51).

Prior to stem cell transplantation the intestines of the patient are sterilized to the extent possible by treatment with two or three broad spectrum antibiotics. Following this treatment the intestine resembles the sterile intestine of the preterm infant. If a patient develops Graft versus host disease one of the first and most prominent symptom is usually severe colitis sharing characteristics with necrotizing enterocolitis in preterm infants. This example thus serves to demonstrate that oral treatment with hBD-2 from the time of stem cell transplantation prevents development of colitis and reduces mortality dramatically.

### Example 12

To determine and assess the efficacy of prophylactic treatment with oral hBD-2 versus cyclosporine in a murine model of acute graft versus host disease following stem cell transplantation.

Materials and Methods

Treatment Regime:

20 female BALB/c mice were irradiated with 4.5 Gy (2×498 sec) at intervals of at least 4 hours on day 0. Bone marrow was harvested: hind limbs harvested from 2 female WT C57BL/6 mice aseptically. Skeletal muscle was removed and epiphysis cut away. Lumen of bone flushed with PBS and cells pelleted and taken up. Harvest of T-cells: spleens from 2 female WT C57BL/6 mice were meshed through a 100 μM cell strainer into a PBS filled dish. Cells were taken up in PBS and transferred into a 50 mL falcon tube and spun down. Cells were taken up in 1 mL PBS and 20 μL of CD4 microbead+20 μL CD8 microbeads per spleen added for incubation for 20 min at 4 degrees C. Cells were washed with PBS and applied to MACS separation in order to positively select CD4+ and CD8+cells. Positively selected T-cells were counted and taken up. Transplantation: directly after the second irradiation, all WT BALB/c recipient mice were injected with 5×1.000.000 BM cells (50 μL i.v. into the retroorbital venous plexus under isoflurane anesthesia. 20 BALB/c mice were treated with 1.2 mg of hBD2/kg BW/day (n=7); 50 mg cyclosporine/kg BW on days 0, 3, 6 and 9 (n=7) or 100 μL PBS per day vial oral gavage (n=6) from day 0-10.

Tests:

The mice were weighed at regular intervals during the study and survival was monitored for 90 days.

Results

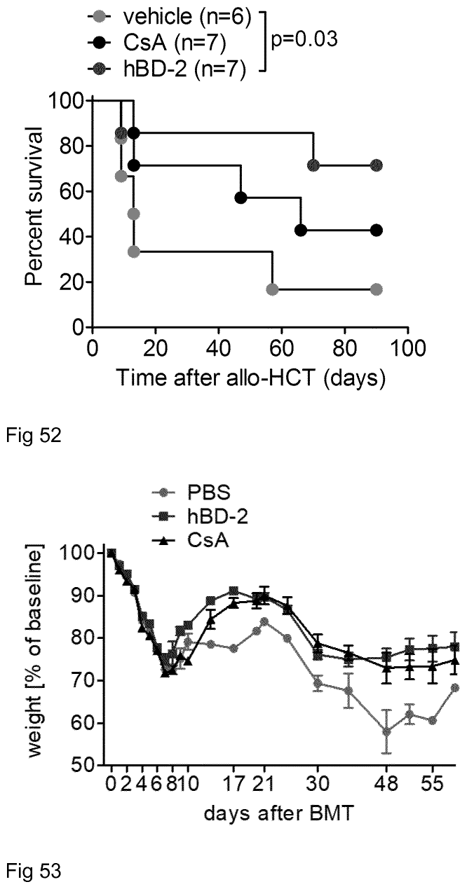

All but one of the PBS treated mice had died by day 90, whereas only 1 of the hBD-2 treated animals had died by day 90 (p=0.03) and 3 animals of the cyclosporine treated animals had died (FIG. 52). The hBD2 and cyclosporine treated mice lost statistically significantly less weight following bone marrow transplantation compared with the PBS treated mice suggesting improved gut health and gut integrity (FIG. 53). Prior to stem cell transplantation the intestines of the patient are sterilized to the extent possible by treatment with two or three broad spectrum antibiotics. Following this treatment the intestine resembles the sterile intestine of the preterm infant. If a patient develops Graft versus host disease one of the first and most prominent symptom is usually severe colitis sharing characteristics with necrotizing enterocolitis in preterm infants. This example thus serves to demonstrate that oral treatment with hBD-2 from the time of stem cell transplantation prevents development of colitis and reduces mortality dramatically.

### Example 13

To determine and assess the efficacy of prophylactic treatment with oral HD5 in a murine model of acute graft versus host disease following stem cell transplantation.

Materials and Methods

Treatment Regime:

22 female BALB/c mice were irradiated with 4.5 Gy (2×498 sec) at intervals of at least 4 hours on day 0. Bone marrow was harvested: hind limbs harvested from 2 female WT C57BL/6 mice aseptically. Skeletal muscle was removed and epiphysis cut away. Lumen of bone flushed with PBS and cells pelleted and taken up. Harvest of T-cells: spleens from 2 female WT C57BL/6 mice were meshed through a 100 μM cell strainer into a PBS filled dish. Cells were taken up in PBS and transferred into a 50 mL falcon tube and spun down. Cells were taken up in 1 mL PBS and 20 μL of CD4 microbead+20 μL CD8 microbeads per spleen added for incubation for 20 min at 4 degrees C. Cells were washed with PBS and applied to MACS separation in order to positively select CD4+ and CD8+cells. Positively selected T-cells were counted and taken up. Transplantation: directly after the second irradiation, all WT BALB/c recipient mice were injected with 5×1.000.000 BM cells (50 μL) i.v. into the retroorbital venous plexus under isoflurane anesthesia. 22 BALB/c mice were treated with 1.2 mg of HD5/kg BW/day; 1.2 mg of hBD-2/kg BW/day or 100 μL of PBS per day vial oral gavage from day 0-10.

Tests:

Survival was monitored for 60 days.

Results

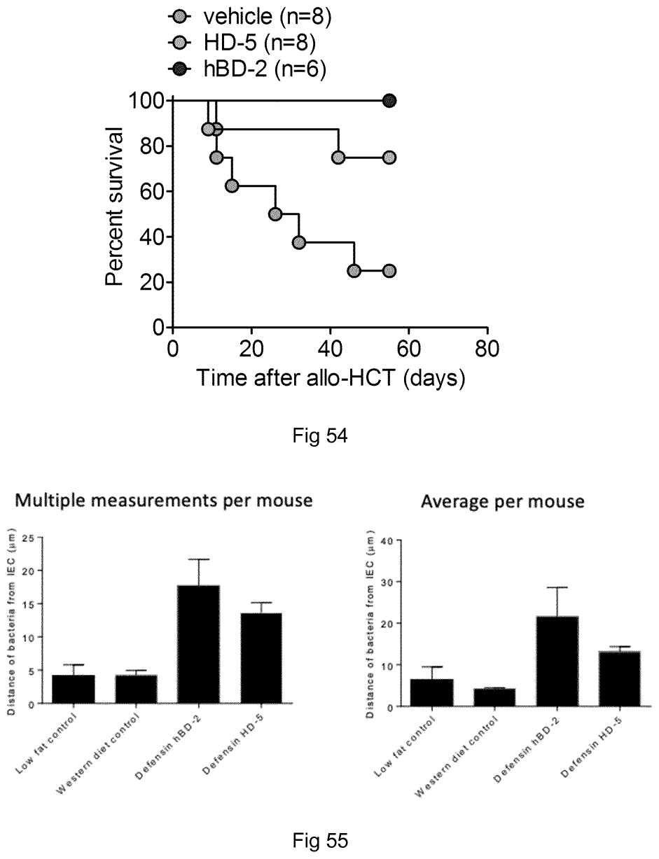

Six out of eight of the PBS treated mice had died by day 60, whereas 2 out of 8 of the HD5 treated animals had died and none of the hBD-2 treated animals had died by day 60 (FIG. 54).

Prior to stem cell transplantation the intestines of the patient are sterilized to the extent possible by treatment with two or three broad spectrum antibiotics. Following this treatment the intestine resembles the sterile intestine of the preterm infant. If a patient develops Graft versus host disease one of the first and most prominent symptom is usually severe colitis sharing characteristics with necrotizing enterocolitis in preterm infants. This example thus serves to demonstrate that oral treatment with hBD-2 from the time of stem cell transplantation prevents development of colitis and reduces mortality dramatically.

### Example 14

Anti microbial effect of orally administered defensins (HD5 and hBD-2).

Method:

Immunostaining of mucins and localization of bacteria by FISH

Mucus immunostaining was paired with fluorescent in situ hybridization (FISH), as previously described in order to analyze bacteria localization at the surface of the intestinal mucosa. Briefly, colonic tissues (proximal colon, 2nd cm from the cecum) without fecal material were placed in methanol-Carnoy's fixative solution (60% methanol, 30% chloroform, 10% glacial acetic acid) for a minimum of 3 hours at room temperature. Tissues were then washed in methanol 2×30 min, ethanol 2×15 min, ethanol/xylene (1:1), 15 min and xylene 2×15 min, followed by embedding in Paraffin with a vertical orientation. Five μm sections were performed and dewax by preheating at 60° C. for 10 min, followed by xylene 60° C. for 10 min, xylene for 10 min and 99.5% ethanol for 10 minutes. Hybridization step was performed at 50° C. overnight with EUB338 probe (5′-GCTGCCTCCCGTAGGAGT-3′, with a 5′ labeling using Alexa 647) diluted to a final concentration of 10 μg/mL in hybridization buffer (20 mM Tris-HCl, pH 7.4, 0.9 M NaCl, 0.1% SDS, 20% formamide). After washing 10 min in wash buffer (20 mM Tris-HCl, pH 7.4, 0.9 M NaCl) and 3×10 min in PBS, PAP pen (Sigma-Aldrich) was used to mark around the section and block solution (5% fetal bovine serum in PBS) was added for 30 min at 4° C. Mucin-2 primary antibody (rabbit H-300, Santa Cruz Biotechnology, Dallas, Tex., USA) was diluted 1:1500 in block solution and apply overnight at 4° C. After washing 3×10 min in PBS, block solution containing anti-rabbit Alexa 488 secondary antibody diluted 1:1500, Phalloidin-Tetramethylrhodamine B isothiocyanate (Sigma-Aldrich) at 1 μg/mL and Hoechst 33258 (Sigma-Aldrich) at 10 μg/mL was applied to the section for 2 h. After washing 3×10 min in PBS slides were mounted using Prolong anti-fade mounting media (Life Technologies, Carlsbad, Calif., USA). Observations were performed with a Zeiss LSM 700 confocal microscope with software Zen 2011 version 7.1. This software was used to determine the distance between bacteria and epithelial cell monolayer, as well as the mucus thickness.

Results:

A highly statistically significant difference in the lysis zone between the intestinal wall and the bacterial population was observed. The distance was small for mice fed both a low fat as well as a western diet, whereas HD5 and in particular hBD-2 had a profound effect on the distance (FIG. 55).

Conclusion:

This experiment demonstrates that orally administered HD5 and hBD-2 in a mouse exerts their microbiota modulating effect at the epithelial surface as if they were produced by the epithelial cells of the mouse themselves and not primarily in the lumen of the intestine as one would expect following oral administration.

### REFERENCES

AlFaleh and Anabrees. Probiotics for prevention of necrotizing enterocolitis in preterm infants. Cochrane Database Syst Rev 2014; 4:CD005496.
Butel et al. Conditions of bifidobacterial colonization in preterm infants: a prospective analysis. J pediatr Gastroenterol Nutr 2007; 44: 577-582.
Campeotto, F. et al. Fecal expression of human β-defensin-2 following birth. Neonatology 2010; 98: 365-369.
Donia, M. S. and Fischbach, M. A. 2015. Small molecules from the human microbiota. Science 349, 1254766
Dorrestein, P. C. et al., 2014. Finding the missing links among metabolites, microbes, and the host. Immunity 40: 824-832.
Gritz and Bandari. The human neonatal gut microbiome: a brief review. Frontiers in pediatrics 2015; 3.
Guaraldi and Salvatori. Effect of breast and formula feeding on gut microbiota shaping in newborns. Front Cell Infect Microbiol 2012; 2: 94
Hanson. Breastfeeding provides passive and likely long-lasting active immunity. Ann Allergy 1998; 81: 523-534.
Jakobsson, H. E. et al. 2014. Decreased gut microbiota diversity, delayed Bacteroidetes colonization and reduced Th1 responses in infants delivered by Caesarian section. Gut 63: 559-566.
Kai-Larsen et al. A review of the innate immune defense of the human foetus and newborn, with emphasis on antimicrobial peptides. Acta Paediatrica 2014; 103: 1000-1008.
Langston et al. Human lung growth in late gestation and in the neonate. Am Rev Respir 1984; 129: 607-613.
Mallow et al. Human enteric defensins. Gene structure and developmental expression. J Biol Chem 1996; 271: 4038-4045.
Manzoni et al. Bovine lactoferrin supplementation for prevention of necrotizing enterocolitis in very-low-birth-weight neonates: a randomized clinical trial. Early Hum Dev 2014; 90: 60-65.
Patel and Denning. Therapeutic use of prebiotics, probiotics and postbiotics to prevent necrotizing enterocolitis: What is the current evidence. Clin Perinatol 2013; 40: 11-25
Puiman, P. J. Paneth cell hyperplasia and metaplasia in necrotizing enterocolitis. Pediatr Res 2011, 69: 217-223.
Richter et al. Influence of gestational age, cesarean section, and type of feeding on fecal human beta-defensin 2 and tumor necrosis factor-alpha. J Pediatr Gastroenterol Nutr 2010; 51:103-5.
Saigal and Doyle. An overview of mortality and sequelae of preterm birth from infancy to adulthood. Lancet 2008; 371:261-269.
Sakurai et al. Extrauterine growth restriction in preterm infants of gestational age<32 weeks. Pediatrics International 2008; 50: 70-75.
Salzman et al. Enteric defensin expression in necrotizing enterocolitis. Pediatr Res 1998; 44: 20-26.
Salzman N H, Underwood M A and Bevins C L, 2007. Paneth cells, defensins, and the commensal microbiota: a hypothesis on intimate interplay at the intestinal mucosa. Semin Immunol 19(2):70-83.
Sangild et al. Invited review: the preterm pig as a model in pediatric gastroenterology. J Anim Sci 2013; 91:4713-29.
Schirmer, M. et al., 2016. Linking the human gut microbiome to inflammatory cytokine production capacity. Cell 167: 1125-1136
Sharma et al. Neonatal gut barrier and multiple organ failure: role of endotoxin and proinflammatory cytokines in sepsis and necrotizing enterocolitis. J Pediatr Surg 2007; 42: 454-461.
Sheng et al. Human β-defensin-3 promotes intestinal epithelial cell migration and reduces the development of necrotizing enterocolitis in a neonatal rat model. Pediatric Researh 2014; 76: 269279.
Siggers et al. Nutritional modulation of the gut microbiota and immune system in preterm neonates susceptible to necrotizing enterocolitis. J Nutr Biochem 2010;
Starner et al. Expression and activity of β-defensins and LL-37 in the developing human lung. J Immunol 2005; 174: 1608-1615.
Stoll and Hansen. Infections in VLBW infants: studies from the NICHD neonatal research network. Semin Perinatol 2003; 27: 293-310.
Strunk et al. Infection-induced inflammation and cerebral injury in preterm infants. Lancet Infect Dis 2014; 14:751-62.
Sweet et al. Two-year outcome of infants weighing 600 grams or less at birth and born 1994 through 1998 Obstet Gynecol 2003; 101: 18-23.
Tirone, C. et al. Correlation of levels of alpha-defensins determined by HPLC-ESI-MS in bronchoalveolar lavage fluid with the diagnosis of pneumonia in premature neonates. Pediatr Res 2010; 68: 140-144.
Trend et al. Levels of innate immune factors in preterm and term mother's breast milk during the 1^{st} month postpartum. British Journal of Nutrition 2016;
Trompette, A. et al., 2013. Gut microbiota metabolism of dietary fiber influences allergic airway disease and hematopoiesis. Nature Medicine 20: 159-168.
Wehkamp J, et al., 2002. Innate immunity and colonic inflammation: enhanced expression of epithelial alpha-defensins. Dig Dis Sci. 47(6):1349-55.
WO 2010/007166
WO 92/06204
WO 95/17413
WO 95/22625
U.S. Pat. No. 5,223,409
WO 2010/007166

1. A method for preventing or treating a mucosal disorder in the gut and/or lung of a preterm infant, the method comprising administering a composition comprising at least one antimicrobial peptide selected from the group consisting of: β-defensins and α-defensins to a preterm infant or to a woman about to give birth to a preterm infant.

2. The method according to any one of the preceding claims, wherein the mucosal disorder is in the gut and is selected from the group consisting of: necrotizing enterocolitis, acute and prolonged diarrhea, and short bowel syndrome, preferably necrotizing enterocolitis.

3. The method according to any one of the preceding claims, wherein the mucosal disorder is in the lung and is selected from the group consisting of: respiratory illness, lung inflammation, respiratory tract infection, respiratory failure, pneumonia, obstructive apnea, bronchopulmonary dysplasia, respiratory distress syndrome, and primary atelectasis.

4. The method according to any one of preceding claims, wherein said β-defensins and α-defensins are selected from the group consisting of hBD-2, truncated hBD-2, hBD-1, hBD-3, hBD-4, HD5, HD6, fragments of defensins and glycosylated defensins.

5. The method according to any one of the preceding claims, wherein said β-defensins and α-defensins are selected from the group consisting of hBD-2, truncated hBD-2, hBD-1, hBD-4, HD5, and HD6.

6. The method according to any of the preceding claims, wherein said β-defensin and α-defensin is hBD-2 and/or HD5.

7. The method according to any of the preceding claims, wherein the mucosal disorder is necrotizing enterocolitis and the defensin is hBD2.

8. The method according to any one of the preceding claims, wherein said composition comprises more than one antimicrobial peptide, such as two antimicrobial peptides, such as three antimicrobial peptides, such as four antimicrobial peptides, such as five antimicrobial peptides.

9. The method according to any of the preceding claims, wherein the composition comprises two defensins, such as for example comprises hBD-2 and HD5.

10. The method according to any one of the preceding claims, wherein said composition is a pharmaceutical composition.

11. The method according to any one of the preceding claims, wherein said antimicrobial peptide further comprises at least one additional moiety selected from the group consisting of a cell penetrating peptide (CPP), an Albumin Binding Moiety (ABM), a detectable moiety (Z), and a half-life extending peptide.

12. The method according to claim 11, wherein the additional moiety is a half-life extending peptide.

13. The method according to claim 12, wherein the half-life extending peptide is a molecule capable of binding to a compound selected from a group consisting of a neonatal Fc receptor (FcRn), transferrin, albumin (HAS), XTEN® or PEG, a homo-amino acid polymer (HAP), a proline-alanine-serine polymer (PAS), or an elastin-like peptide (ELP), hyaluronic acid, a negatively charged highly siasylated peptide such as the carboxy-terminal peptide (CTP) of chorionic gonadotropin (CG) β-chain, human IgG, and CH3(CH2)ₙCO— wherein n is 8 to 22.

14. The method according to any one of the preceding claims, wherein the antimicrobial peptide is administered in combination with surfactants and/or prebiotics and/or probiotics and/or tryptophane, and/or glucocorticoids and/or antibiotics and/or immunosuppressants and/or GLP-2 and/or GLP-2 analogs or any combination thereof.

15. The method according to any one of the preceding claims, wherein said antimicrobial peptide is administered to a preterm infant at least every other day, one time a day, such as at least twice a day, such as at least three times a day, such as at least four times a day, such as five times a day or continuously.

16. The method according to any of the preceding claims, wherein at least one defensin is administered to the preterm infant starting on the date of birth.

17. The method according to any of the preceding claims, wherein at least one defensin is administered to the mother prior to giving birth to a preterm infant.

18. The method according to any one of the preceding claims, wherein said administration is oral, buccal, sublingual, rectal, vaginal, intratracheal, intrapulmonary, intranasal, intracranial, subcutaneous, intravenous, dermal or transdermal.

19. The method according to any one of the preceding claims, wherein said administration is oral.

20. The method according to any one of the preceding claims, wherein said administration is intrapulmonary, intratracheal or intranasal.

21. The method according to claim 20, wherein said intrapulmonary, intratracheal or intranasal administration is by an inhaler, nebulizer, or vaporizer.

22. The method according to any of the preceding claims, wherein the preterm infant has a birth weight of 1,500 grams or less, such as 1,000 grams or less.

23. An antimicrobial peptide for use in a method of treatment according to any of the preceding claims.

24. A defensin polypeptide for use in a method of treatment according to any of the preceding claims.

25. Use of a defensin polypeptide for the manufacture of a medicament for use in the treatment of a disorder as defined in any of the preceding claims.


SEQUENCE LISTING (16 sequences)

SEQ ID NO: 1 - protein, 36 aa, Homo sapiens
DHYNCVSSGGQCLYSACPIFTKIQGTCYRGKAKCCK

SEQ ID NO: 2 - protein, 41 aa, Homo sapiens
GIGDPVTCLKSGAICHPVFCPRRYKQIGTCGLPGTKCCKKP

SEQ ID NO: 3 - protein, 44 aa, Homo sapiens
GIINTLQKYYCRVRGGRCAVLSCLPKEEQIGKCSTRGRKCCRRK

SEQ ID NO: 4 - protein, 37 aa, Homo sapiens
ELDRICGYGTARCRKKCRSQEYRIGRCPNTYACCLRK

SEQ ID NO: 5 - protein, 32 aa, Homo sapiens
ATCYCRTGRCATRESLSGVCEISGRLYRLCCR

SEQ ID NO: 6 - protein, 32 aa, Homo sapiens
AFTCHCRRSCYSTEYSYGTCTVMGINHRFCCL

SEQ ID NO: 7 - protein, 40 aa, Mus musculus
KINNPVSCLRKGGRCWNRCIGNTRQIGSCGVPFLKCCKRK

SEQ ID NO: 8 - protein, 30 aa, Homo sapiens
ACYCRIPACIAGERRYGTCIYQGRLWAFCC

SEQ ID NO: 9 - protein, 29 aa, Homo sapiens
CYCRIPACIAGERRYGTCIYQGRLWAFCC

SEQ ID NO: 10 - protein, 30 aa, Homo sapiens
DCYCRIPACIAGERRYGTCIYQGRLWAFCC

SEQ ID NO: 11 - protein, 33 aa, Homo sapiens
VCSCRLVFCRRTELRVGNCLIGGVSFTYCCTRV

SEQ ID NO: 12 - protein, 37 aa, Homo sapiens
LLGDFFRKSKEKIGKEFKRIVQRIKDFLRNLVPRTES

SEQ ID NO: 13 - protein, 690 aa, Homo sapiens
GRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 14 - protein, 48 aa, Homo sapiens
GRRRSVQWCAVSQPEATKCFQWQRNMRKVRGPPVSCIKRDSPIQCIQA

SEQ ID NO: 15 - protein, 130 aa, Homo sapiens
KVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 16 - protein, 37 aa, Homo sapiens
PVTCLKSGAICHPVFCPRRYKQIGTCGLPGTKCCKKP